(19) United States
(12) Patent Application Publication
(10) Pub. No.: US 2020/0232961 A1
(43) Pub. Date: Jul. 23, 2020

(54) CHARACTERIZATION AND APPLICATION OF POLYMERS FOR IN VIVO RELEVANT DRUG ABSORPTION CHARACTERIZATION IN VITRO

(71) Applicant: THE REGENTS OF THE UNIVERSITY OF MICHIGAN, Ann Arbor, MI (US)

(72) Inventors: Gregory E. Amidon, Portage, MI (US); Patrick D. Sinko, Ann Arbor, MI (US)

(21) Appl. No.: 16/648,151

(22) PCT Filed: Sep. 18, 2018
(86) PCT No.: PCT/US18/51605

§ 371 Date: Mar. 17, 2020

Related U.S. Application Data
(60) Provisional application No. 62/560,552, filed on Sep. 19, 2017.

(51) Int. Cl. G01N 33/15; A61K 9/00; A61K 31/80

(52) U.S. Cl. CPC G01N 33/15; A61K 31/80; A61K 9/0053

(57) ABSTRACT

The disclosure provides a synthetic polymer that mimics the passive absorption kinetics of the human intestinal tract. More particularly, disclosed is a silicone-based polymer, e.g., poly(dimethyl siloxane), poly(dimethyl silicone) and poly siloxane, that meets the requirements of a robust, semipermeable, and in vivo-relevant membrane for use in an in vitro method for measuring the absorption of a chemical compound, such as a therapeutic, e.g., a small-molecule or a biologic, that can be expected to reflect the absorption properties of the chemical compound in the vertebrate gastrointestinal tract, thereby providing an assessment of absorption of the compound in the vertebrate GI tract.

### CROSS-REFERENCE TO RELATED APPLICATIONS

This application claims the priority benefit of provisional U.S. Patent Application No. 62/560,552, filed Sep. 19, 2017, herein incorporated by reference.

### STATEMENT OF GOVERNMENT INTEREST

This invention was made with government support under grant no. HHS F223201510157C, awarded by the U.S. Food and Drug Administration. The government has certain rights in the invention.

### FIELD

The disclosure relates to the characterization of chemical compounds using an in vitro mimic of the vertebrate intestinal tract and, more particularly, to the dissolution and absorption characteristics of chemical compounds and materials used in such characterizations of chemical compounds.

### BACKGROUND

The United States Pharmacopeia (USP) established some of the most ubiquitous dissolution methodologies, equipment, and standards used today around the world. In 1950, the USP's only official test for tablet and capsules was the disintegration test, but this test was known to be indirectly related to drug bioavailability and product performance. In 1962, dissolution testing was beginning to gain support as a more discriminating test for drug bioavailability and drug product performance. In 1968, the USP1 basket-stirred flask apparatus was introduced and by 1975, through a collaborative effort of industry and government, there was convincing evidence that the USP1 could provide an in vivo-in vitro correlation. These findings provided momentum for a movement away from an in vitro-in vivo USP bioavailability standard. The USP1 was complemented by the USP paddle apparatus (USP2) in 1978 and, at the same time, the USP adopted the sink condition (3 times volume required to saturate a solution (about 500 mL-1000 mL; typically, 900 mL), in simple aqueous media to eliminate the use of enzymes in simulated intestinal fluid) and 50 rpm paddle speed to maximize product discrimination. Between 1985-1990, after the advent of more complex dosage forms (e.g., enteric coated, extend/delayed release), the USP implemented an official system for accounting in vivo-in vitro correlations (levels A, B, C, D).⁵² This development provided a framework to compare how well a drug product performed relative to a pharmacokinetic clinical study. The trend of using dissolution testing as a method to discriminate between formulations, which is the primary use in industry to date, is contrary to the original intentions of the implementation of dissolution testing. This development was well intentioned and rational, due to the need to quantify formulation consistency and to ensure product safety and efficacy for the general public. However, this disclosure resulted from experiments that questioned the utility of the USP dissolution test, with one focus being to make the dissolution test more physiologically meaningful. Typical efforts along these lines attempt to make the content of the dissolution medium biomimetic, while few efforts focus on the apparatus or in vivo mechanical/hydrodynamic forces.⁵³⁻⁶²

For orally administered drugs, in vivo relevant (ivR) dissolution methodology examines the drug product in an in vitro experimental system that strives to accurately simulate the critical parameters of the in vivo environment and processes of the vertebrate, e.g., human, gastrointestinal tract. The information is important in focusing pharmaceutical development pipelines on therapeutics, e.g., drugs such as biologics and small-molecule chemicals, that exhibit physico-chemical properties compatible with delivery to, dissolution in, and absorption by the vertebrate GI tract at efficacious yet safe dosages.

Various in vitro systems have been used in the past to attempt to integrate absorption kinetics into dissolution methods, such as the octanol-water system, Caco-2 cell membrane permeability assay, and parallel artificial membrane permeability assay (PAMPA). These systems fall short, however, in providing realistic human GI absorption kinetics in a robust and facile in vitro setting.

Biphasic systems or organic-solvent-based absorption systems (OSAS) have been utilized in the pharmaceutical industry to incorporate a passive absorption compartment into ivR in vitro dissolution systems.⁷⁻¹¹ Systems such as octanol-water, however, can be challenging to use. The boundary that occurs between the aqueous and organic phases is more dynamic than a physical barrier.¹² Large agitations can create mixing between the organic and aqueous layers, which can result in a poorly defined interface. A significant challenge in OSAS systems is adjusting the absorptive surface area to dissolution volume ratio to modulate the interfacial mass transfer rate to accurately simulate human oral absorption.

Other popular systems for simultaneous dissolution and absorption studies are the cell-based membrane systems. Yamashita et al. have done extensive work with Caco-2 and MDCK II monolayer cell membranes in side-by-side diffusion cells in an effort to include absorption kinetics in dissolution tests for the purpose of in vitro-in vivo correlation and evaluation of drug candidates during the early discovery phase of drug development.¹³⁻¹⁶ They also have examined formulation effects (micelle transport and food effect) in vitro on Caco-2 membrane transport.¹⁷⁻¹⁹ The donor and receiver volumes (on the order of 1-10 mL) make these assays attractive to industry and academia for higher throughput permeation studies (used in well-plate assays), in addition to containing biological drug transporters in some systems that allow for active transport to occur in an in vitro environment. However, Caco-2 cell membrane assays to date have not attempted to achieve the correct membrane surface area to dissolution volume ratio to replicate human absorption rates. On balance, there are significant challenges in using Caco-2 or similar systems. Caco-2 cells are known for their significant inter-laboratory variability, which requires an external system of validation to verify any results generated using Caco-2 permeability assays. In the recent past, a clone of Caco-2 (TC7) was developed to increase the consistency of Caco-2 assays. Cell based systems require prolonged periods of time for cells to culture prior to use, unlike a synthetic membrane like PDMS or PAMPA. Additionally, drug molecules tend to be retained in the Caco-2 membrane when the drug exhibits poor solubility, and the hydrodynamic boundary layer severely affects the permeation of drug molecules through the membrane.²⁰⁻²¹

PAMPA is another tool that was developed in the late 1990s to improve and expedite the evaluation of new chemical entities in terms of permeability and estimated oral absorption rates. The development of PAMPA focused on high-throughput screening, which gives PAMPA a distinct advantage over Caco-2 or other cell-based assays. This advantage is particularly valuable in the pharmaceutical industry, where the rapid pace of early discovery-phase pharmaceutical development demands robust, repeatable, and fast/real-time analytical techniques in data analysis.²² There are a range of structural configurations for different routes of drug administrations, i.e., oral, brain, skin, and different combinations of polymer/organic/phospholipid phases) for PAMPA systems.²³⁻²⁶ PAMPA has even been used in parallel studies with Caco-2 assays to determine effects of reflux and efflux transporters on permeability and oral absorption.²⁷ These characteristics make PAMPA a powerful tool for permeability screening and mechanism determination, but a poor method for incorporating realistic vertebrate, e.g., mammalian such as human, GI absorption kinetics into dissolution methods. PAMPA suffers the same limitations as cell-based assays where interfacial area is determined artificially by the micro well-plates used in the assay, and the available volumes for dissolution are not scaled to match the human GI situation. The hydrodynamic boundary layer can also significantly affect the permeation of lipophilic drugs.²⁸

Computer-aided design (CAD) and additive manufacturing have been used since the 1980s to reduce the cost, effort, and production time of prototype models for engineered parts. CAD models of a part are made in a variety of commercially available software packages and exported into a “.stl” file (Standard Tessellation Language or STereoLithographic file). The stl file provides coordinates for triangular planes which represent small portions of surfaces of the CAD model. Higher tessellation translates to more resolution the surface has, which ultimately leads to smoother or more detailed printed parts. One of the most popular additive manufacturing techniques is stereolithography (SL), which is one method within the colloquial term “3D printing”. SL uses photosensitive monomer resins that polymerize when exposed to ultraviolet light. The penetration of ultraviolet light is shallow in the liquid monomer resin, thus the polymerization reaction occurs near the liquid surface. Once the photopolymerization is complete, the build substrate is lowered into the reservoir of liquid monomer resin and then the photopolymerization process is repeated over the slice of the two-dimensional cross section until the part is completed. Another common additive manufacturing technique is fused deposited modeling (FDM). FDM relies on hot melt extrusion, specifically, heating the material 0.5° C. above the melting temperature and then depositing it through a movable head. The material rapidly cools after the extrusion and cold welds in place to the adjoining layers. The resolution of the additive manufacturing device is described in terms of voxels, or three-dimensional pixels.⁶⁶⁻⁶⁸

CAD, FDM, and SL have recently begun to enter the field of pharmaceutical science in the form of drug delivery systems, such as modified and immediate release tablets, caplets, and disks.⁶⁹⁻⁷⁷ While there appears to be interest in using additive manufacturing to develop new ways to control dose weight and dissolution properties, there is no information about using additive manufacturing to improve the science of dissolution itself.

In view of the disadvantages and limitations of existing technologies for characterizing the dissolution and absorption properties of orally administrable compounds, e.g., therapeutics, a need continues to exist in the art for assessing the dissolution and absorption properties of such compounds in a manner that reflects their in vivo behavior upon oral administration.

### SUMMARY

The disclosure provides investigations establishing the use of a PDMS membrane, such as found in the context of an ultra-thin, large-area poly(dimethylsiloxane) membrane diffusion cell or UTLAM PDMS, to overcome the experimental challenges encountered in other in vitro permeation assays/methods. A properly selected material can yield a membrane that mitigates or eliminates the experimental challenges found in other in vitro absorption systems. The selection of an optimal polymer membrane is more than a question of lipophilicity, however. There are in vitro aspects that must be considered as well, such as erosion, degradation, and physical stability. Water intrusion/swellability, creation of aqueous pores, diffusion of drug, diffusion of polymer degradation products, micro-environmental pH changes, diffusion through pores, hydrogen/hydroxide ion transport into the membrane causing micro-environment pH changes, osmotic effects, convection, adsorption/desorption, and partitioning behavior may all affect mass transport.²⁹

An in vivo relevant (ivR) in vitro model of drug absorption uses physiologically relevant fluids (e.g., pH, volume, temperature, buffer, buffer capacity, surfactants), hydrodynamic conditions (e.g., shear, advection), and mass transfer rates (e.g., diffusion, permeation to simulate the absorption process) in an in vitro system can better simulate in vivo performance than current and common compendial dissolution methodologies.^{1-4, 92} Disclosed herein, in relevant part, is the design, fabrication, and evaluation of a new in vitro device that incorporates new knowledge of the human gastrointestinal (GI) tract from a unique clinical study performed in humans and computation fluid dynamics simulations.^{50, 51}

An ultra-thin, large area poly(dimethylsiloxane) membrane diffusion cell (UTLAM) (PDMS) simulates the passive diffusion mechanism of the human oral absorption pathway (duodenum and jejunum).⁹² It is a single compartment vessel that incorporates a hydrofoil impeller to reduce bulk fluid shear rates while maintaining axial mixing that promotes particle re-suspension. It is contemplated that various embodiments of the UTLAM diffusion cell will emulate different human GI tract compartmental segments using buffer and pumping parameters similar to those used in artificial stomach and duodenum (ASD) and gastrointestinal simulator (GIS) devices.

Since there is no significant convective passive drug transport through the human GI tract, polymers that were known to have convective transport behaviors such as sieving or size exclusion were eliminated from consideration for the biomimetic in vitro membrane. For robust and simple in vitro mass transport analysis, the membrane needs to be stable across the pH spectrum and be unaffected by the solution conditions present in the donor and receiver compartments confining donor and receiver fluids, respectively. The donor fluid or compartment contains the initial concentration of the compound of interest while the receiver fluid or compartment is separated, at least in part, therefrom by the polymer and the receiver fluid or compartment initially lacks the compound of interest. Selecting the polymer to not include highly reactive functional groups ensures physical and chemical stability. Polymers containing carboxylic anhydrides, ketal-, and ortho-ester functional groups were excluded because they are known to be some of the most reactive functional groups. The type of drug (acid or base) and the drug's interaction with the membrane can contribute to degradation or hydrolysis of the membrane. Acidic and basic drugs can autocatalyze degradation by changing the micro-environment pH in the polymer.²⁹ These reasons resulted in the exclusion of hydrogels and other common control-released polymers. While employed in other pharmaceutical applications, the use of these types of polymers in in vivo Relevant (ivR) absorption applications would be problematic due to the diffusion coefficient becoming dependent on the aqueous content in the membrane, and because such polymers exhibit a time-dependent interfacial surface area. Silicone-based polymers, including poly (dimethyl siloxanes), poly-dimethyl silicones, and poly-siloxanes, are identified as suitable polymers for use in the ivR methodologies disclosed herein. An exemplary silicone-based polymer, poly(dimethyl siloxane) (PDMS), is characterized herein because it exhibits the desirable characteristics of this class of polymer, i.e., it is non-swelling, has non-interconnected porosity, is lipophilic/organophilic, and is pH stable. In addition, PDMS is easy to fabricate, is inexpensive, and is widely available.

PDMS as an in vitro biomimetic analog of the passive drug absorption process in the human gastrointestinal (GI) tract was assessed. PDMS is biomimetic because of similarities to the GI tract in small molecule transport, such as mechanism, ionization selectivity, and lipophilicity.

The disclosure provides improved in vitro methods for measuring the absorption characteristics of orally administrable compounds such as therapeutics, including biologics and small-molecule compounds. As noted above, the methods rely on the identification of a material that closely mimics the in vivo behavior of the vertebrate gastrointestinal tract based on significant similarities of the structural and functional properties of the material to the structural and functional properties of the vertebrate GI tract. That material is a silicone-based polymer, e.g., poly (dimethyl siloxane), which is shown herein to possess the structural characteristics of a stable polymer exhibiting unconnected pores establishing gut-like porosity without deteriorating in the presence of aqueous fluids. The disclosure also provides methods of producing such materials, e.g., in the form of polymeric membranes of various sizes and thicknesses, useful in assessing the absorption characteristics of compounds in a simple and cost-effective manner.

Using physiologically relevant fluids (e.g., pH, volumes, temperature, buffer, buffer capacity, surfactants), hydrodynamic conditions (e.g., shear, advection), and mass transfer rates (e.g., diffusion, permeation to simulate the absorption process) in an in vitro system can better simulate in vivo performance than current and common compendial dissolution methodologies. The disclosure provides a synthetic polymer that closely approximates the passive absorption kinetics of the human intestinal tract. More particularly, disclosed is a silicone-based polymer that meets the requirements of a robust, semipermeable, and in vivo-relevant (i.e., ivR), in vitro membrane. By measuring the drug permeability in the disclosed membrane system, its capacity to act as an ivR membrane was demonstrated for a variety of drugs that span the lipophilicity spectrum.

The disclosure is drawn to an in vitro method of measuring absorption of an orally administrable compound as a method of assessing the absorption of the compound in the vertebrate gastrointestinal tract, the method comprising: (a) contacting a silicone-based polymer with an orally administrable compound in vitro; and (b) measuring the absorption rate of the compound. In some embodiments, the polymer is a poly (dimethyl siloxane), a poly di-methyl silicone or a poly siloxane polymer. In some embodiments, the polymer is a poly (di-methyl siloxane) (PDMS) polymer. In some embodiments, the absorption measure comprises: (a) determining the aqueous initial concentration of compound before exposure to the polymer (e.g., poly (dimethyl siloxane) (PDMS)); (b) measuring the rate of appearance of compound after exposure to the polymer in a receiver compartment; and (c) using a scaled surface area of the polymer and scaled volume available for diffusion to assess the absorption of the compound in the vertebrate gastrointestinal tract. Typically, assessment of the absorption of the compound will reveal that the absorption of the compound by the polymer simulates the absorption of the compound by the vertebrate intestinal tract. Embodiments of this aspect of the disclosure are contemplated wherein the polymer (e.g., poly (dimethyl siloxane)) comprises pores having an average pore diameter of 0.4 to 0.9 nanometers, such as a pore diameter that is 0.8 to 0.9 nanometers. The pore dimensions are found over a broad temperature range (see FIG. 3A), such as at 37° C. In some embodiments, the polymer (e.g., poly (dimethyl siloxane)) has an average molecular weight between 6,000 and 70,000 daltons. In some embodiments, the polymer (e.g., poly (dimethyl siloxane), poly (di-methyl silicone) or poly siloxane) is derivatized with end groups comprising at least one methyl end group, at least one hydroxyl end group, at least one vinyl end group, or at least one hydrogen end group, wherein the polymer is derivatized with an end group at each end of the polymer. Embodiments are provided wherein the compound is hydrophilic, or hydrophobic. In some embodiments, the compound is negatively charged; in some embodiments, the compound is positively charged; in some embodiments, the compound is uncharged. Also contemplated are embodiments wherein the compound is a Biopharmaceutics Classification System (BCS) Class I or Class II compound exhibiting high permeability or a BCS Class III or Class IV compound exhibiting low permeability. In some embodiments, the polymer (e.g., poly (dimethyl siloxane)), comprises pores stable in size for at least 193 days. In some embodiments, the polymer (e.g., poly (dimethyl siloxane)) exhibits an elastic modulus of at least 0.2 MPa, or exhibits an elastic modulus no greater than 2.50 MPa. Some embodiments of the method comprise a polymer (e.g., poly (dimethyl siloxane)) comprising a cross-linking agent between 3% and 25% weight percent. In some embodiments, the polymer (e.g., poly (dimethyl siloxane)) is in the form of a membrane.

Other features and advantages of the disclosure will become apparent from the following detailed description. It should be understood, however, that the detailed description and the specific examples, while indicating embodiments of the disclosed subject matter, are given by way of illustration only, because various changes and modifications within the spirit and scope of the disclosure will become apparent to those skilled in the art from this detailed description.

### BRIEF DESCRIPTION OF THE DRAWING

FIG. 1. A schematic diagram of the rotating diffusion cell.⁵ (A) Cross section showing the outer housing of the diffusion cell, which is freely rotating. (B) The inner housing, which is connected to the support structure and is non-moving. Two large fillets within the inner housing allow for media to cascade over the edge creating a well mixed environment. (C) shows the upper portion of the support structure that is attached, e.g., using clamps, to thereby hold the diffusion cell in place during an experiment. Within the support structure is a hollow tube that enters the inner housing, allowing for sampling mechanisms (probes or pipettes) to access the inner chamber. (D) identifies the membrane interphase.

FIG. 2. Chemical structure of Sylgard 184® base material³².

FIG. 3. (A) Measuring the PDMS pore diameter as a function of temperature demonstrated that very small changes in void morphology were accurately measured. (B) The temperature was cycled between R_{T} and the target temperature to see if there was any evidence of permanent thermal history. PDMS recovered both R_{T} Ps lifetime and signal intensity after being exposed to different temperatures. Four distinct regions were fit, i.e., −240° C. to −122° C. (glassy), T_{G}=−110° C.±8° C.; −100° C. to −47.5° C. (glass transition region), Tₘₑₗₜ=−44° C.±7° C.; −25° C. to 25° C. (rubbery plateau/flow); 25° C. to 150° C. (viscous flow). The flat region in this Figure is actually a thermal plateau that originates from the competitive phenomena of linear thermal expansion of the pore and increased oscillation of the pore due to increased thermal energy. The Ps particle was annihilated upon the pore contracting during an inward oscillation so the true maximum pore size cannot truly be measured. This is typically described as the “roll-over” region. It was assumed that any changes in the morphological structure caused during experimentation would be detectable, but lower temperatures are contemplated for such characterizations. The application of this system, to replicate human body temperatures, requires that analysis be conducted at 37° C. The PALS measured T_{G} and Tₘ are consistent with reference³⁰ and reference³⁸, respectively, for PDMS. N=1 membrane, 2-3×10⁵ events on average per run at each temperature.

FIG. 4. (A) The primary goal in utilizing PDMS was to partition drug molecules from drug- and drug product-containing phase into a reservoir compartment at magnitudes comparable to rates observed in humans. At equilibrium partitioning, it was expected that drug or other components that partition into the membrane would not accumulate in PDMS pores, thereby affecting the Ps lifetime. A highly lipophilic drug, ibuprofen, was chosen because a large amount of drug would be retained by the membrane, increasing the probability that a change in the lifetime could be observed. A change in Ps lifetime was not observed. (B) A statistical change was observed in the positron lifetime. N=1 membrane per condition, 3-5×10⁶ events on average per run.

FIG. 5. (A) pH-distributed partition coefficients in HCl buffer, pH 2.0, and 50 mM phosphate buffer, pH 6.50, with model drug ibuprofen. Three different concentrations of curing agent in membranes were measured to see if the curing agent provided any lipophilic difference to the final cured membrane. There was no practical difference among the partition coefficients of the varied concentrations of curing agent in the membranes. N=5 membranes per curing agent condition per pH condition. Nₜₒₜₐₗ=30. (B) Varying the amount of curing agent of PDMS was expected to provide a simple method for modulating membrane permeability and was expected to add another mechanism of permeation control. The extreme concentrations of curing agent were used to elucidate differences in permeability behavior. There was a statistical difference according to a two tailed t-test (p<0.02) with N=6 for the low extreme concentration of 3.2% and N=8 for the high extreme concentration of curing agent. However, the practical difference in the context of the ivR methodology was negligible. This also implies that permeability was not significantly affected by minor process variations during fabrication. The permeand was the model drug, ibuprofen, under intrinsic non-ionizing conditions (pH=2.0 HCl) with receiver phase 50 mM phosphate, pH 6.5, 37° C., 150 rpm in the rotating diffusion cell. (C) Distribution partition coefficient experiments were conducted to confirm that Equations 5-6 were valid in PDMS. These equations are useful in predicting absorption rate over a range of pH.

FIG. 6. A correlation of the predicted octanol-water intrinsic non-ionized partition coefficient and the measured PDMS intrinsic non-ionized partition coefficient. It was determined that there was no practical difference between the partitioning of most drugs into the PDMS membrane at 12 and 24 hours, in which the 12-hour time-point more accurately represented “equilibrium” in the context of the time scale for typical human GI motility patterns. Partition coefficient was calculated using equation 7. N=5 membranes per drug except dipyridamole where N=1. Nₜₒₜₐₗ=41.

FIG. 7. (A) The correlation between octanol-water and PDMS partitioning was established to allow for a prediction directly from the chemical structure to the permeation through a PDMS membrane at any nominal thickness. Looking at FIG. 6 and FIG. 7A, a clear path to an initial rational based dissolution-simultaneous absorption (ivR) experiment can be designed for any drug molecule. (B) PDMS permeability scales with thickness (1/hₘₑₐₙ) and the permeability of a drug can be predicted from the K×D product at any thickness. The K×D product was calculated at each thickness and the average was used in P=(K×D)/h. N=1 membrane per thickness (data). The K×D relationship measured in FIG. 7A was used to predict ibuprofen-PDMS permeability (Equation 17). Ibuprofen under intrinsic non-ionizing conditions (pH=2.0 HCl) with receiver phase 50 mM phosphate, pH 6.5, 37° C., 150 rpm in the rotating diffusion cell. Gray bands on each trend indicate the positive 95% confidence interval. (C) PDMS is resistant to transporting polar entities, and it was reasonable to suspect that the polar surface area of a molecule would influence the overall diffusivity of the species. PDMS diffusion is also a function of the molecule's lipophilicity towards PDMS. Ketoprofen deviates from the visually observable trend in FIG. 6, but when the polar surface area is considered, ketoprofen aligns as expected. Based on the strong power law relationship, D_{PDMS} is inversely proportional to the polar surface area of the molecule.

FIG. 8. (A) Shows the effect of 0.01, 0.1, 1 mm/s strain rates on three different PDMS blends (25, 9.1, 3.2)% curing agent by mass, or (3:1, 10:1, 30:1) mass base:mass curing agent ratios. It was determined that the smallest strain rate would be suitable for the remaining mechanical characterizations. N=5 per strain rate per PDMS blend. Nₜₒₜₐₗ=45. (B) When the concentration of curing agent is varied, the elastic modulus can be manipulated. This property is important for the eventual processing and manufacturing of PDMS into thin hollow tubes or sheets for drug-partitioning processes. PDMS made using the hexane drop-cast method, shows a trend similar to that observed in⁴⁶. A critical point exists beyond which the addition of curing agent no longer stiffens the material. Comparing the overall magnitude of the elastic modulus between the simple mix and the hexane drop-cast method, hexane produces a lower elastic modulus polymer. N=5 PDMS cylinders per concentration, L/D about 0.5. Nₜₒₜₐₗ=30. (C) PDMS was prepared in hexane, and allowed to cure at various temperatures. PDMS can be made stiffer by increasing the cure temperature. These data result from using 9.1% mass curing agent PDMS. N=5 membrane cylinders per temperature, L/D about 0.5. Nₜₒₜₐₗ=15.

FIG. 9. (A) The void size was measured via Ps-ALS at R_{T} (20° C.) to show that the void structure had long term stability. There was no statistical difference between pore sizes measured at 3 days and 193 days post-casting, and there were 3-5×10⁶ events, on average, per run. (B) Higher curing temperature was examined to see if the pore structure formed at higher temperature was physically different than when cured at R_{T}. Using Ps lifetime as an indirect measure or analog for pore size, the void structure was unaffected by the curing temperature. N=1 membrane per temperature, with 3-5×10⁶ events, on average, per run. (C) To gauge the effect of process variability on the void structure, Ps lifetimes were measured over a wide range of mass % of curing agent, where 0% is the neat base material in its viscous fluid state. There was no significant change in Ps lifetimes over the range of curing agent mass % studied. N=1 PALS run per concentration, 3.6-7.2×10⁵ events per run.

FIG. 10. HPLC standard curves are shown (partition coefficient and solubility).

FIG. 11. UV-Vis Dip Probe standard curves are illustrated (permeation in the rotating diffusion cell).

FIG. 12. Limit of detection (LOD) and limit of quantification (LOQ) for HPLC standard curves.

FIG. 13. Example of estimating the dead zone volume created by a hydrofoil impeller in a flat bottom tank. This calculation was estimated for anchor, paddle, and hydrofoil impellers in cone, flat, and dish bottom vessels.

FIG. 14. Examples of different dissolution vessels simulated in COMSOL CFD mixer module package.

FIG. 15. The effects of average shear and average velocity were examined in the context of the expected volumes for a typical in vivo-relevant test as well as three different configurations of dissolution vessels. Only the hydrofoil data have lines connecting data points because this impeller style was of the greatest interest to the development of the device.

FIG. 16. (A) The velocity flow field from a COMSOL CFD experiment in the UTLAM dissolution bowl stirred at 120 rpm by the 0.5·Dtank hydrofoil. (B) The corresponding shear rate profile was the same computational experiment as (A).

FIG. 17. A comparison of the flow field between two candidate impeller configurations and a traditional USP 2 paddle.

FIG. 18. Computational study using the Algebraic Plus turbulent model to solve the CFD in COMSOL to determine the proper ratio of impeller diameter to vessel diameter. Designing the system for flexibility, the ratio that allows for the greatest range of shear rates was determined to be the most desirable configuration. These quantities were solved using a Reynolds Averaged Naiver-Stokes (RANS) Equation in COMSOL. These values represent the steady-state condition of the fluid.

FIG. 19. A-C) Computational study using the Algebraic Plus turbulent model to solve the CFD in COMSOL to determine the proper stirring speed of the impeller based on the desired bulk fluid performance. Vertical lines indicate the minimum stirring speed for stable turbulent flow based on the Reynold Number calculation. Simulations must be performed at stirring speeds above this minimum to be physically valid. D) Visualizations of the shear and velocity profile for the 130 mL flat-bottom UTLAM dissolution bowl stirred at 60 rpm.

FIG. 20. CAD models of two cross sections of the UTLAM diffusion cell. Gray indicates a surface 1 not in contact with the cutting plane. Light green is the dissolution bowl 2, pink is the central hub 3, which acts as the attachment point for all other components, blue is the shear to normal force converter 4, which protects the PDMS UTLAM from being displaced during assembly, yellow is the membrane support hub 5 where the membrane sits in both the release/transfer vessel and the diffusion cell, and orange is the absorption chamber 6. V_{dissolution chamber}=131 mL (not total capacity); V_{absorption chamber}=105 mL. **plot** This plot describes the “efficiency” of the design choices we have made by showing the design variables overlayed. The x-axis is the literal height of the liquid dissolution media. Because the amount of liquid is governed by a combination of height and area, the initial volume of fluid for dissolution can be related to the absorptive surface area to dissolution volume ratio through the fluid height. Initial conceptions of the hydrofoil hub and blades required the fluid height to be a minimum of 15 mm tall to cover the blades, and a gap is required between the bottom of the hub's shaft and the membrane to prevent wear on the membrane/particles getting caught in the clearance and damaging the membrane. In this one convenient plot, the absorption rate criteria based on the geometry and orientation of the diffusion cell components can be rapidly evaluated. The farther to the left on the x-axis, the higher the absorption rate and the closer the volumes of dissolution media are to the in vivo situation. Farther to the right on the x-axis, the dissolution volume approaches compendial amounts and the lower the maximum absorption rate will be. At the qualitative intersection of the two lines is where the volume is minimized for enhanced absorption performance. Further reduction in volume (if possible) will improve absorption rate and ultimately the flexibility/capability for ivR absorption of the UTLAM diffusion cell.

FIG. 21. A) PVA spun out of deionized water coats a 100 mm silicon wafer. The thickness profile is described using ellipsometry. Error bars are standard deviation of the mean. B) Results of the AFM study showing the real trace output from the soft-tapping AFM on the surface of the PDMS- and PVA-coated silicon wafer. The red (middle), blue (top), and green (bottom) dots are schematic representations of the dotted lines in the output trace to show how the height of the plateau caused by the PDMS was calculated. The plot of membrane thickness as a function of rotational speed shows signs of error in sample preparation due to the appearance of an apparent asymptotic boundary in thickness appearing in higher solution concentration samples and a reduction in sensitivity to the rotational velocity and solution concentration parameters (compared to values in the literature).

FIG. 22. A) A room-temperature-prepared PDMS membrane was measured via AFM in both the surface contact and free-standing configurations. B) Thickness characterized using SEM of PDMS spun out of hexane coating PVA on silicon at varying mass fractions in solution (analog to solution viscosity) and at varying terminal rotational velocities. C) 25 wt % PDMS solution spun at 4000 rpm post liquid nitrogen cryo-fracture sample preparation. D) 75 wt % PDMS Solution spun at 1000 rpm post liquid nitrogen cryo-fracture sample preparation. Images taken on a Tescan MIRA3 FEG Scanning Electron Microscope. E) A study to determine if there is permanent thermal history imparted to PDMS when exposed to liquid nitrogen for prolonged periods of time. The motivation for this study was that PDMS films prepared above the glass transition temperature presented a mixed mode of failure during sample preparation for atomic force microscopy (AFM). Significant elastic deformation occurred prior to overload failure and the strain (change in length) was maintained by strong surface interactions between the PDMS and the silicon wafer. Cooling the PDMS samples below the glass transition temperature would ensure that only brittle fracture occurred. Surface features were located on an already prepared wafer. They were measured using AFM at room temperature, and then again after 6 minutes of exposure to liquid nitrogen (allowing the sample to return to room temperature). In both the full-surface-contact type of morphology and the free-standing (no contact between PDMS and silicon) condition, the liquid nitrogen cooling step did not produce plastic deformation through thermal expansion. This showed that virgin samples could be cooled below the glass transition temperature and fractured for cross-sectional imaging in the SEM without influencing the soft elastic PDMS.

FIG. 23. Equilibrium partition coefficient measurement of ibuprofen into 10 mm diameter spheres of VeroClear resin from the J750 Printer. Results show that the partitioning affinity for ibuprofen in the VeroClear resin is higher than that of the PDMS membrane (K=65 2 pH 2 in PDMS; K=86 pH 2 in VeroClear). Three vials, each containing one 10 mm diameter sphere per pH, were kept in a temperature-controlled oven for 3 days. 0.4 mL was sampled from the aqueous solution at each time point and the media was not replaced. All solutions were well below solubility of ibuprofen at the pH being tested. Initial and final pHs were measured; however, final pH was plotted because no titrations were performed during the partition experiment.

FIG. 24. A) The content uniformity of each dosing was monitored to ensure that each experimental dose of the uncompressed formulation contained a comparable amount of drug to the intended amount of 162 mg per dose. A mass balance was calculated between the endpoint of the experiment at one hour and after an additional hour where 100 mL of acetonitrile was added to solubilize any undissolved drug and enable the extent of release in one hour to be accurately measured. B) Critical results from the USP 2 monophasic dissolution experiments performed at pH 6.5 in 50 mM phosphate buffer at 37° C. Two of more distinct groups emerge from this screening process, as seen in A). Compositions 1-12 are identified in FIG. 43. N=3 dissolution experiments per formulation.

FIG. 25. The average of the three experimental runs for each of the twelve formulations. The USP 2 does not contain an absorptive phase, and therefore only the dissolution profile could be presented. Each formulation contains the same amount of ibuprofen. Error bars are the sample standard deviation.

FIG. 26. Main Effect plots for the USP II device. Plots show the effect of each factor (excipient) if the factor (explanatory variable) is present or not on the response variable (dissolution rate, Cmax, AUC). The larger the deviation from the central line in either direction, the more significantly that factor influences the response variable. However, for the purposes of this disclosure, the importance is to be able to see if each dissolution test method detects each factor the same and if the relative performances are the same. If they do not, then contemplation of the physical-chemical ramifications of using a particular compendial methodology can be elucidated by people competent in the science. When compared to the biphasic dissolution experiment, these main effects plots demonstrate that the addition of in vivo-relevant conditions significantly changed the response variables, and the factors which produced a significant effect on those response variables.

FIG. 27. Results of a biphasic dissolution experiment are presented (main response variables). The experiment was conducted using the USP 2 device and a 200 mL/200 mL biphasic 1-Octanol/Water composition. Due to the addition of the organic absorption phase, various rates and amounts can be calculated for the organic side alone. However, since the comparison is to the USP 2 monophasic device, the biphasic experiments are only compared in the aqueous dissolution side and over the same length of time (60 minutes).

FIG. 28. The average of the three experimental runs for each of the twelve formulations produced using the biphasic dissolution experiment. The top plot is the aqueous donor phase where the drug powder was introduced through a sample port allowing direct access to the aqueous phase. The bottom plot is the 1-octanol receiver phase where no drug was present at time zero, but accumulated as drug dissolved in the aqueous phase. Each formulation contained the same amount of ibuprofen. Error bars are the sample standard deviation.

FIG. 29. A) Basic physical-chemical information used in the COMSOL CFD and MATLAB dissolution program. B) The particle size distribution of the ibuprofen used in the experiments as determined by dynamic light scattering (DLS), which was used in the MATLAB code to predict dissolution of ibuprofen with a real time change of particle size. The raw data was fit using a high order polynomial (spline fit) and then fractionated into particle size bins to create the particles in the simulation, as the statistical lognormal fits inadequately represented the data from DLS (plot with star symbols). The attempt to statistically fit a lognormal function to the DLS particle size distribution. The as-calculated-fit curve was created using the standard treatment of the data to fit a lognormal probability density function, and the manual fit curve was obtained starting with the as-calculated fit but then performing a reduction of the sum of squared residuals (chi square optimization) until an apparent minimum residual was found. (Black dots are the experimental data, the line with highest number fraction at the lowest particle diameter is standard statistical procedure fit, the line with intermediate (about 0.017) number fraction at the lowest particle diameter is the Chi Square procedure fit.)

FIG. 30. Velocity profile in USPII dissolution apparatus operating at 50 rpm rotational speed produced using the more accurate k-ε turbulence model. This is more computationally expensive to run so only the prime candidate from the CFD screening study stirring conditions was chosen (50 rpm). This condition was used to run the experiments in all dissolutions of the formulations 1-13.

FIG. 31. Logarithmic shear profile for USPII dissolution apparatus. operating at 50 rpm rotational speed. The model described for FIG. 30 was used.

FIG. 32. Velocity profile in UTLAM dissolution apparatus operating at 50 rpm rotational speed using the more accurate k-ε turbulence model. This is more computationally expensive to run so only the prime candidate from the CFD screening study stirring conditions was chosen (50 rpm). This condition was used to run the experiments for pure ibuprofen, but due to the high partitioning affinity of the prototype's material, only 1 experiment was recorded.

FIG. 33. Logarithmic shear profile for UTLAM dissolution apparatus, operating at 50 rpm rotational speed. The UTLAM demonstrates a much more uniform velocity and shear profile, which indicates better mixing than the compendial USP 2 device. Experimental observations confirm that drug particles were constantly moving, and not aggregating at the bottom of the tank.

FIG. 34. Demonstration that using the improved dissolution equation that considers the hydrodynamic forces in the experimental apparatus (using hydrodynamic parameters generated from the COMSOL CFD (k-ε turbulence model)), accurately predicts dissolution performance and was demonstrated in the USP II compendial device. A) The hydrodynamic parameters calculated using a k-ε turbulent model in COMSOL for the USP II paddle apparatus. B) Proof that the MATLAB model (Sherwood Number+Shear factor with accounting for surface pH using the Ozturk model) is valid by demonstrating the prediction of the USP II dissolution of ibuprofen. C) Comparing the relevant bulk average hydrodynamic parameters between the USP II and the UTLAM diffusion cell.

FIG. 35. Direct comparison of dissolution of 162 mg of ibuprofen at 37° C. stirring at 50 rpm with compendial paddle. The comparison is between the standard USP II equipment and the SL printed VeroClear model of the USP II bowl. Both experiments use the same PTFE-coated steel paddle. This demonstrates the effect of the high partitioning kinetics expected from the VeroClear resin. The dissolution rate is significantly increased in the presence of high partition rates.

FIG. 36. The ability to re-use membranes was examined. Experiment 1 (series 1) used the virgin PDMS UTLAM and the expected j-shaped curve (indicating a small lag time) was observed. Experiments 2 and 3 (series 2 and 3, respectively) were conducted using the same experimental conditions except for the membrane, which was the membrane that was used in experiment 1, but washed with deionized water and methanol between experiments. The permeability measured from the pseudo-steady state region decreases with reuse, but the initial concentration decreases as well. This indicates that there is retained drug in the membrane or apparatus itself, which then is extracted by the new dissolution media in each experiment.

FIG. 37. Ibuprofen partitioning in the UTLAM and rotating diffusion cell (RDC) systems. The RDC was used in the chemical characterization of PDMS. The results show the dependence of the absorption rate on pH, with acidic conditions leading to significantly higher absorption rates for ibuprofen, consistent with expectations.

FIG. 38. MATLAB mass balance simulations of the UTLAM experiments. Using the Sherwood Number determined diffusion layer model including shear factor. The plots show a 180-minute simulation of the dissolution and absorption process for 37° C., 50 rpm, pH_{Aq. Donor initial}=6.5, PH_{Aq Receiver initial}=8.0, buffer strength=50 mM, buffer: phosphate, dose: 162 mg Ibuprofen, Volume_{Donor}=130 mL, Volume_{Receiver}=100 mL, sampling volume=2 mL (1 mL sacrificed to waste, 1 mL filtered, media replaced in both phases).

FIG. 39. Variation of blade thickness was illustrated by CAD drawings of the model hydrofoils used to simulate different impeller blade thicknesses.

FIG. 40. CAD drawings of the model hydrofoils used to simulate different angles of attack of the impeller blades. The blades were attached to the same point on the shaft.

FIG. 41. Changing the stirring direction with a hydrofoil impeller does significantly affect the fluid flow pattern in the COMSOL simulations, which is expected to affect particle resuspension behavior. The bulk fluid shear rate and velocity, however, were practically the same.

FIG. 42. A summary of the blade thickness, angle of attack, stirring direction, and change-in-volume studies. A) Volume average shear rate increases parabolically as thickness increases. The net effect is very small, considering the advantages increased thickness brings to actual fabrication of the impeller. B) Changing the angle of attack has a small effect on the bulk properties (shear and velocity) but does significantly affect the flow pattern. C) A similar conclusion can be drawn as in B) when the direction of rotation is reversed. D) Due to the possibility of small changes in volume in each compartment during a GIS-style experiment, it was important to know how significantly the volume average shear rate would change. 25 mL was considered to be 2-5 times more volume change than what would be expected in a GIS-style experiment. This simulation demonstrates that there would not be a significant enhancement to dissolution through increased shear rate even over significant volume drops.

### DETAILED DESCRIPTION

Through a combination of intubation studies (pressure wave motility of segments of the GI tract, gastric contents and pH), real-time magnetic resonance imaging (MRI) manometry (real-time free water flow, motility patterns), and computational fluid-dynamic simulations (CFDS) of peristaltic fluid and mass flow, the first scientifically derived criteria for orally administered dissolution testing has been achieved. With the ability to understand the distinct segmental nature of the gastrointestinal tract, it is possible to capture the most critical parameters (pH, fluid volume, shear rates, secretion rates, intercompartmental transfer rates, antero-retrograde flow rates, and the like) in each segment and in between segments and use these parameters to develop an apparatus that accurately simulates the current understanding of gastrointestinal dissolution processes. See Table 1. These measured and predicted values governed the design, simulation, evaluation, and implementation of the UTLAM devices disclosed herein. It is expected that UTLAM devices will be connected in series to form an artificial organ or organ system, such as an artificial stomach and duodenum or to form a gastrointestinal stimulator, where the design of concatenated UTLAM devices will be influenced by the average residence time of compounds in, e.g., the duodenum and/or the jejunum.

TABLE 1

| HUMAN | | |
|---|---|---|
| PARAMETER | CLINICAL, LITERATURE, SIMULATION VALUE | |
| AQUEOUS | 35 ML ± 7 ML (FASTED) | 242 ML ± 9 ML (2 MIN POST 240 ML GLASS |
| VOLUME IN | | OF WATER DOSING) |
| STOMACH² | | |
| TOTAL AQUEOUS | 5-159 ML (RANGE, | 15-264 ML (RANGE, 12 MIN POST-WATER |
| VOLUME IN | FASTED) | DOSE) |
| LOWER GI² | 43 ML ± 14 ML (FASTED) | 92 ML ± 24 ML (12 MIN POST 240 ML |
| | | GLASS OF WATER DOSING) |
| | | 15-172 ML (RANGE, 45 MIN POST-WATER |
| | | DOSE) |
| | | 77 ML ± 15 ML (45 MIN POST 240 ML |
| | | GLASS OF WATER DOSING) |
| BULK FLUID | S*_{USP II} < 250-500 | S*_{HUMAN GI} = 10-25 |
| PROPERTIES¹⁹⁻²¹ | RE_{SHEAR USP II} < 0.25-0.5 | RE_{SHEAR HUMAN GI} < 0.0007-0.003 |
| (CFDS) | | |
| PH IN STOMACH⁷ | 1.1-7.47 (RANGE, | 1.1-7.39 (RANGE, FED) |
| | FASTED) | 4.04 (MEAN, FED) |
| | 2.50 (MEAN, FASTED) | 3.95 (MEDIAN, FED) |
| | 2.25 (MEDIAN, | |
| | FASTED) | |
| PH IN | 1.71-7.57 (RANGE, | |
| DUODENUM⁷ | FASTED) | |
| | 4.93 (MEAN, FASTED) | |
| | 4.91 (MEDIAN, FASTED) | |
| PH IN JEJUNUM⁷ | 2.2-6.75 (RANGE, | |
| | FASTED) | |
| | 5.55 (MEAN, FASTED) | |
| | 5.62 (MEDIAN, FASTED) | |

Disclosed herein are in vitro methods for measuring the absorption of a compound by a type of polymeric material that may be in the form of a membrane of various thicknesses, wherein the in vitro absorption measurements are in close agreement with the absorption characteristics of the compound in the vertebrate gastrointestinal tract, thereby providing an in vitro assessment of the in vivo absorption characteristics of a given compound in the vertebrate intestinal tract. The material is a poly-silicone polymer such as poly (dimethyl siloxane), poly (dimethyl silicone) or poly siloxane that provides a material of stable structure comprising unconnected pores establishing a porosity closely mimicking the porosity of the vertebrate GI tract, such as the human gastrointestinal tract. The measurement and assessment methods disclosed herein will accelerate efforts to identify compounds such as therapeutics (e.g., small molecule compounds and biologics such as peptides and proteins) that exhibit desirable absorption characteristics in the vertebrate gastrointestinal tract. In addition, the methods will facilitate efforts to characterize known therapeutics and allocate such compounds to administration regimens where their absorption characteristics would be useful. With respect to the devices disclosed herein, construction is aided by the use of various forms of 3D printing. The resolution of the three-dimensional printers used in the experiments disclosed herein range from 16 μm to 85 μm for the SL resin printers (Projet 3500 HD Max/Stratasys J750) and 178 μm-500 μm for the FDM ABS printer (Stratasys Dimension Elite). The ability to rationally design dissolution methodologies and equipment that are relevant to physiologic dissolution are now possible because of rapid prototyping simulated parts (vessels and impellers) from computational fluid dynamics simulations, and informed by clinical studies.

One of the main kinetic processes involved in gastrointestinal dissolution is absorption, but this process is rarely captured in dissolution testing. Poly(dimethyl siloxane) (PDMS) membranes have been demonstrated to be adequate biomimetic analogue for the passive oral absorption pathway in human beings.⁹² The ability to implement a biomimetic polymer membrane is highly desirable for the experimental advantages over similar organic solvent based systems. To achieve absorption rates for BCS class I, II, and III compounds that are within the expected physiological norm, PDMS membranes must be thin and have a large surface area. One method to produce highly homogenous, uniformly thick, ultra thin, large surface area membranes is to use a spin coater. Spin coaters are known for the ability to produce high quality, homogenous films at scales as low as single nanometers.^{47, 78, 79} PDMS membranes have been produced that were larger than 5 cm² and approximately 100 nm thick.⁴⁷ A similar method was used to generate PDMS membranes that simulate passive oral absorption in humans using a sacrificial polyvinyl alcohol (PVA) film for release instead of gelatin. PVA's solvent (deionized water) is orthogonally soluble to the PDMS solvent (hexane), which makes it a convenient choice for a water-soluble sacrificial release layer. PVA was also found to dissolve in about two hours at room temperature (about 60 nm×25 πcm² PVA). Upon release from the silicon wafer, the PDMS can become wrinkled if a weight is used to submerge the wafer. These wrinkles relax in deionized water at room temperature over several hours uninfluenced or manually relaxed with the aid of a tweezers in seconds. To measure the thicknesses of the PVA, ellipsometric measurements sufficed, but because of the target thicknesses of the biomimetic PDMS UTLAM, ellipsometry of the bilayer film was not possible. Scanning electron microscopy, after a liquid nitrogen fracture, was used to analyze the thickness of the PDMS film that had been spun out of hexane onto a prepared PVA-coated silicon wafer. When the PDMS UTLAM was utilized for diffusion experiments, it was released from the silicon wafer in a long, wide, and shallow tray containing deionized water, which allows the PDMS to float to the surface of the water, completely unsupported by any structure other than itself. The PDMS UTLAM is mechanically strong enough to be handled, but because the UTLAMs are semi-self-adherent, it is difficult to flatten a membrane should it come into contact with itself. Therefore, PDMS UTLAMs are transferred from the release/transfer vessel into the diffusion cell using a part of the diffusion cell disclosed herein.

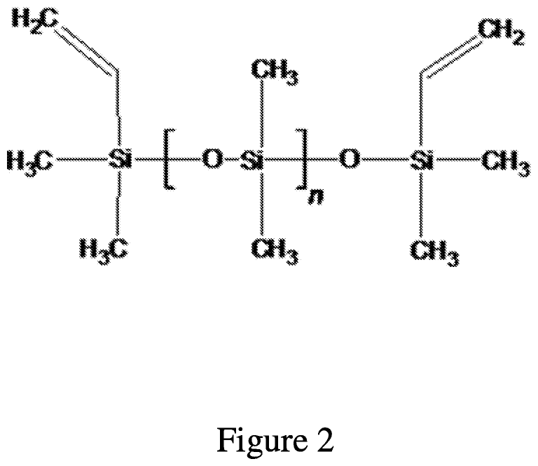

An exemplary polymer is poly(dimethyl siloxane) (PDMS), which was commercialized in 1943 by the Dow Corning company and was obtained for these studies as the Sylgard 184 elastomer kit.³⁰ This kit contains two components, the polymer base and the polymer curing agent. The polymer base contains 60% dimethylsiloxane, which is dimethylvinyl terminated, 30%-40% dimethylvinylated and trimethylated silica, and 1-5% tetra(trimethylsiloxy)silane. The base material is viscous, with a η_{base}=5000 cS. The curing agent contains dimethyl methylhydrogen siloxane that, through a platinum catalyst, initiates a step-wise polymerization using a hydrosilation reaction at the vinyl groups in the base material.³¹⁻³² At room temperature (R_{T}; 20° C.) PDMS forms a transparent, colorless, elastomeric polymer, see FIG. 2. The density of the cured polymer (at 10:1, mass of base: mass of curing agent) is 1.03-1.05 g cm⁻³ at 25° C. Over 9.1% catalyst yields more hydrogen-bonded silicon (SiH) polymer, which is a soft filler material; under 9.1% curing agent or catalyst, there are more vinyl unreacted end groups and physical entanglement becomes a more relevant mechanism in the formation of the gel, e.g., membrane, because of hanging chains. The polymer is 100% cross-linked at a 10:1 monomer:curing agent (m:m) ratio. The addition of silica filler can be used to adjust the viscosity of the compositions. The glass transition temperature of PDMS is −127° C., which implies that at R_{T} the polymer is in the viscous-flow regime.³⁰ PDMS has a critical molecular mass of M_{c}=24,500 daltons, an average molecular weight of chain segment between intermolecular junctions of Mₑ=12,293 daltons, and reptation tube diameter of dₜ=78.6 Å.³⁰ PDMS obeys steady state and pseudo-steady-state Fick's law for diffusion and is impermeable to hydrochloric acid, phosphate buffer salts, protonated aminophenones, charged organic molecules, inorganic ions, and does not readily transport water and mineral oil. Ethanol, however, is transported without modifying the membrane.³³.

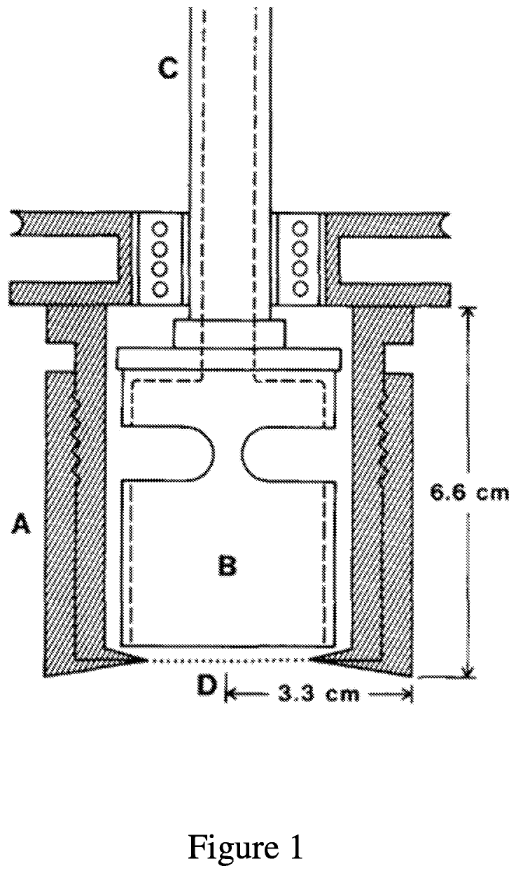

For orally administered drugs, dissolution methodologies should examine the drug product in an in vivo Relevant (ivR) in vitro experimental system that accurately simulates the critical parameters of the in vivo environment and kinetic processes of the human GI tract. The ivR hypothesis is that using physiologically relevant fluids (e.g., pH, volumes, temperature, buffer, buffer capacity, surfactants), hydrodynamic conditions (e.g., shear, advection), and mass transfer rates (e.g., diffusion, permeation to simulate the absorption process) in an in vitro system can better simulate in vivo performance than current and common compendial dissolution methodologies.¹⁻⁴ This disclosure establishes that PDMS can be used to replicate the passive absorption kinetics of the human GI so that it can be applied to a device that meets the criteria for ivR dissolution. PDMS was characterized using a rotating membrane diffusion cell (FIG. 1) to determine if it meets the requirements for a robust, semipermeable, and in vivo-relevant in vitro membrane. By measuring the fundamental transport properties of the membrane system, its potential to act as an ivR membrane can be easily evaluated for a variety of drug molecules.

The data disclosed herein indicate that it is feasible to construct physically viable membranes with permeation properties adequate to simulate oral absorption for a wide variety of drug molecules, typically those considered to be high permeability (BCS Class 1 and 2).

Mudie et al. laid the experimental ground work for ivR absorption⁶, the goal of which is to construct an in vitro system in which the partition rate constant (k_{abs}_{_{in vitro}}) is approximately equal to the in vivo absorption rate constant (k_{abs}_{_{in vivo}}).

$$\begin{matrix}{k}_{{abs}_{i n vivo}} \approx {k}_{{abs}_{i n vitro}} & ( 1 ) \\ {P}_{i n vivo} \frac{{A}_{G - I}}{{V}_{i n vivo}} = {P}_{octanol} \frac{\frac{Ao}{w} interface}{{V}_{i n vitro}} & ( 2 )\end{matrix}$$

Renaming the variables to the membrane-based absorption system, Sinko et al propose:

$${P}_{i n vivo } \frac{{A}_{G - I}}{{V}_{i n vivo}} = {P}_{PDMS} \frac{{A}_{membrane}}{{V}_{i n vitro}} \qquad ( 3 )$$

### Where:

k_{abs}_{_{in vivo}}=the 1^{st} order absorption rate coefficient in humans
k_{abs}_{_{in vitro}}=the 1^{st} order absorption rate coefficient in the in vitro apparatus
P_{in vivo}=the measured/predicted permeability coefficient in humans
A_{G-I}=surface area available for absorption in the human GI

$${A}_{\frac{o}{w} interface} = the area of contact between the organic and aqueous phases$$

=the area of contact between the organic and aqueous phases
V_{in vivo}=the effective aqueous volume of dissolution within the human GI
P_{PDMS}=the permeability coefficient of drug in PDMS
A_{membrane}=surface area of membrane available for transport
V_{in vitro}=the effective volume in the dissolution apparatus which dissolution occurs (volume of the drug-donating phase)

Where Equation (3) allows for human absorption kinetics, either measured or predicted, to be replicated in vitro by scaling the A_{in vitro}/V_{in vitro} to make k_{abs in vivo} k_{abs in vitro}.

Ten molecular probes were used to evaluate the transport pathways and properties used to predict human oral absorption rates. The transport pathways through PDMS (bulk/pore) are analogous to transcellular (TCDT) and paracellular (PCDT) drug transport pathways. PDMS PCDT was assessed using positronium annihilation life-time spectroscopy (PALS) and partition experiments; TCDT using diffusion and partition experiments. PALS determined that PDMS pores were uniform (D˜0.85 nm), isolated, and void volume was unaffected by drug accumulation after equilibrium partitioning. Therefore, there is no PCDT or convective flow through PDMS. A strong linear correlation exists between predicted octanol-water partition coefficients and PDMS partition coefficients (Log P_{PDMS}=0.736×Log P_{O-W}−0.971, R2=0.981). The pH-partition hypothesis is confirmed in PDMS using ibuprofen over pH 2-12. Diffusivity through PDMS is a function of lipophilicity and polar surface area

$$K \times {D}_{P D M S} = 4 .46 \times {10}^{- 8} \times {e}^{2 .91 \times {LogP}_{P D M S}} ( {R}^{2} = 0.9 \; 63 ) and \\ K \times {D}_{P D M S} = 4 .61 \times {10}^{- 6} \times {( \frac{P S A}{{K}_{P D M S}} )}^{- 1.20} ( {R}^{2} = 0.9 \; 73 ) .$$

Varying the mass % of curing agent changed the lipophilicity and diffusivity (p<0.02), but not practically (K×D=2.23×10⁻⁵ cm²s⁻¹ versus 2.60×10⁻⁵ cm²s⁻¹), and does affect elastic modulus (3.2%=0.3 MPa to 25%=3.2 MPa).

As the experiments described in the following examples show, PDMS does not have interconnected porosity as measured by beam PALS. No drug was quantified in the PALS void volume (as measured by a change in lifetime), nor was any change in mass of the membrane measured when soaked in pure water. The effective diffusive flow of drug appears to transport within the densely packed-domains in the polymer network. PDMS is pH stable, as shown in the Log D_{PDMS} experiments for ibuprofen over a pH range of 2.0-12.0. The K×D product successfully predicted ibuprofen permeability over a 500 μm difference in thickness of PDMS membrane. The use of the K×D product (Diffusivity) to predict PDMS-drug permeability is valid at any thickness at which PDMS membranes are currently produced. Large area (>5 cm²), ultra-thin (1 μm) membrane fabrication is possible and is an exemplary type of geometry useful for characterizing absorption rates of pharmaceuticals that are comparable to human GI absorption rates.⁴⁷

PDMS, however, can be fabricated to have a 3 dimensional surface area, which is capable of accommodating even larger surface area to volume ratios without sacrificing the physiologically relevant volumes required by the ivR methodology.⁴⁸ The pure diffusion coefficients in PDMS are significantly slower (about 10²) than those in water, but the true diffusion coefficients for PDMS must account for the partitioning behavior into PDMS and the polar surface area of the solute molecule

$$( K \times D ∝ {K}_{P D M S} ; K \times D ∝ \frac{1}{PSA} ) .$$

Knowing the thickness-independent permeability (i.e., the diffusivity) (K×D) behavior allows for ivR modeling of the absorption kinetics using an in vitro test. PALS characterization at room and physiologic temperature of the PDMS membrane shows that the physical structure of the membrane is not significantly affected by any processing or experimental parameters that a membrane would be exposed to in ivR dissolution and absorption experiments. Dissolving the PDMS components in hexane produces softer (lower Young's modulus) membranes than PDMS that is fabricated with no solvent. The Young's modulus can be modulated approximately between 0.3 MPa and 2.3 MPa by changing the amount of curing agent added to the base material during fabrication. Even though high temperature curing is limited to about 60° C. in hexane during polymerization (T_{Boiling hexane}=about 70° C.), exposing the polymer solution to temperatures above RT can result in up to a 0.5 MPa increase in the Young's modulus. While these mechanical differences may be significant in fabrication of the in vitro absorption component, the mechanical differences do not affect the drug permeation performance significantly.

The following examples are included to demonstrate embodiments of the disclosed subject matter. Those of skill in the art will, in light of the present disclosure, appreciate that changes can be made in the specific embodiments which are disclosed and still obtain a like or similar result without departing from the spirit and scope of the disclosure.

### EXAMPLES

### Example 1

Materials and Methods

Materials

Materials used in the experiments disclosed herein include an Agilent 1100 High Performance Liquid Chromotography (HPLC), an Eclipse Plus C 18 Column (3.5 μm×4.6 μm×150 mm), Acetonitrile (EMD Millipore, HPLC grade), deionized water (Milli-Q purified), trifluoroacetic acid (Fisher Scientific, Optima Grade), triethylamine (Fisher Scientific, Optima Grade), methanol (Fisher Scientific, HPLC grade), Ibuprofen (Albemarle Lot No. 2050-0032F), Progesterone (Sigma Aldrich, CAS 57-83-0), Benzoic Acid (Fisher Scientific CAS 65-85-0), Metoprolol Tartrate (Sigma Aldrich, CAS 56392-17-7), Caffeine (Sigma Aldrich, CAS 58-08-2), Atenolol (Sigma Aldrich, CAS 29122-68-7), Ketoprofen (TCI Tokyo, Japan, CAS 22071-15-4), Hydrochloric acid buffer pH 2.0 (USP guideline), Acetate Buffer 5.0 (USP guideline), Phosphate buffer pH 6.5 (USP guideline), Sodium Hydroxide Buffer pH 12 (NaOH+KCl), Poly(dimethyl siloxane), (Sylgard 184 elastomer kit, Dow Corning), Hexane (Fisher, reagent grade), Instron uniaxial press, Fisher-Scientific accuSpin micro17 Centrifuge, Wenesco Inc., and HP1212-D cure plates with glass covers.

Generic Membrane Casting Procedure

Sylgard 184 base was weighed in a glass container and moved to a vacuum chamber where a −750 mbar vacuum was pulled for 25 minutes to remove gas. Separately, an appropriate amount of Sylgard 184 curing agent was weighed on an analytical balance. A 1:1 ratio (total mass:volume) of hexane was measured in a graduated cylinder. The hexane was used to dissolve the catalyst component and then was added to the container containing the base polymer. Manual mixing was done until the base was completely dissolved (easily observed by a change in the index of refraction). An appropriate volume of solution was drop cast into polyethylene weigh boats using a pipette. The PDMS solution cured at the desired temperature and time with solvent evaporating into a lab hood.

Strain Rate Effect in PDMS Mechanical Samples

Three strain rates were tested: 1, 0.1, 0.01 mm s⁻¹ strain rates in uniaxial compression.

PDMS Elastic Modulus and Cure Temperature Sample Preparations

PDMS cylinders were prepared at 3 mm thickness and 6 mm diameter. For the elastic modulus samples, the ratio of the polymer base to curing agent was varied (3, 7, 10, 15, 20, 30):1. For cure temperature samples, the base to curing agent ratio was 10:1. Each sample was cured at a different temperature for 9 days. Samples cured above 40° C. were allowed to cure at R_{T} until the hexane was evaporated (<1 day) until the film was semi-solid, and then the remainder of the nine-day cure was completed. This prevented boiling hexane from forming bubbles within the sample.

Effect of Curing Temperature on Elastic Modulus

The curing temperatures studied were 20° C., 40° C., and 60° C. Five samples were prepared using a 6 mm diameter surgical punch (L/D (length/diameter) about 0.5). The modulus was calculated from the linear slope on the compression stress-strain curve at a strain rate of 0.01 mm s⁻¹.

Elastic Modulus Versus Composition Ratio

Five samples were prepared using a 6 mm diameter surgical punch (L/D about 0.5). The modulus was calculated from the linear slope on the compression stress-strain curve.

HPLC Methods

For acidic drugs, 0.085% v/v trifluoroacetic acid was used in both water and acetonitrile. For basic drugs, 0.1% v/v triethylamine was used in both water and acetonitrile. See Table 2 for details. For HPLC Standard Curves, limit of detection (LOD) and limit of quantitation (LOQ), a five-point standard curve (two-fold dilution per step) was created for each drug in the buffer used for the experiment. See Example 11 for LOD and LOQ.

TABLE 2
Description of the HPLC methods, including
the mobile phase composition used and the
average elution time of the molecule.

| | Mobile Phase | |
|---|---|---|
| | Composition | Average Elution Time |
| Drug | (Acetonitrile/H₂O) | (Minutes) |
| Progesterone | 60/40 | 6.63 |
| Ibuprofen | 60/40 | 5.04 |
| Benzoic Acid | 29/71 | 4.52 |
| Ketoprofen | 49/51 | 4.48 |
| Caffeine | 17/83 | 2.62 |
| Atenolol | 19/81 | 4.52 |
| Metoprolol Tartrate | 41/59 | 4.28 |

Partition Coefficient Measurements

For each drug, five membranes were prepared. Each membrane was prepared with 10 parts base to 1 part curing agent and cured at 20° C. for at least 72 hours. Once cured, the membranes were sectioned using a template and razor blade. The dimensions of the perimeter and thickness were measured using a caliper to determine the volume of membrane. After determining membrane density, subsequent volume measurements were made using the density relationship. Stock solution was distributed to 5 sample vials with 1 membrane-free vial to serve as a control. The time zero point was measured from the blank vial and time points and 1 mL samples were taken at 12 and 24 hours. The collected samples were assayed in duplicate by HPLC.

Distribution Partition Coefficient Measurements

This experiment was conducted in a manner similar to the experiment described in “Partition Coefficient Measurements”, above. The model drug, ibuprofen, was exposed to R_{T} 13 mM HCl at pH 2.00, 50 mM acetate at pH 5.00, 50 mM phosphate at pH 6.50, and 39 mM NaOH at pH 12.00.

Non-Ionized Thermodynamic Solubility Determination at 37° C.

Five 1.5 mL centrifuge tubes were labeled and prepared with 1 mL of the appropriate non-ionizing buffer (see Table 5). Solid drug in powder form was added to each individual vial and then vortexed. The addition of drug and vortex mixing was repeated until there was visible undissolved drug powder present. The centrifuge tubes were then put into a hot box where they remained at an aqueous temperature of 37° C. (set point 43° C.). The internal temperature was determined by measuring the temperature of a “blank” tube in the rack. Once the internal temperature reached 37° C., the tubes were held at 37° C. for 48 hours. The tubes were removed from the hot box and centrifuged at 17,000G for 3 hours. The tubes cooled below 37° C. during the centrifugation so the tubes were re-inserted into the hot box and allowed to reach 37° C. over the course of 1 hour. Supernatant was then extracted directly from the tube in the hot box without disturbing the pellet. The supernatant was appropriately diluted for HPLC analysis. The limit of quantification and limit of detection were calculated to ensure the validity of the dilution scheme and use of the standard curves (Equation S49-S51, see Example 11).

Rotating Membrane Diffusion Cell Experiments

Thickness of the sample membrane was measured using a caliper at the center of the membrane and then at four additional points within the circumference of the membrane in the region which was exposed to the drug-saturated aqueous phase. The initial mass of the membrane was weighed prior to drug exposure. A recirculating bath warmed the beaker containing the donor aqueous suspension (0.9 mM sodium dodecyl sulfate) of drug to 37° C. A rotating membrane diffusion cell was utilized, as shown in FIG. 1.⁵ Seventy mL of the appropriate receiver phase was then added into the inner chamber of the diffusion cell. This receiver phase was a medium that ionized the drug once drug passed completely through the membrane, creating sink conditions and preventing reverse transport. For drugs that were non-ionizable, the receiver phase and donor phase were compositionally equivalent except for any surfactant and drug, which was solely present in the donor phase. The dip probe was calibrated in situ for each experiment and recorded one measurement every 60 seconds (five spectra averaged per measurement). Donor phase volume of 250 mL was added to the warmed jacketed beaker, and then raised into contact with the diffusion cell. Air that was present in between the membrane and the aqueous phase was removed using a syringe. The diffusion cell was rotated at 150 rpm during the experiment. An additional procedure was used for permeation measurements of metoprolol tartrate, as this compound is a salt form of the drug metoprolol. Tartrate salt is acidic and upon dissociation in the aqueous environment, the pH will undergo an acidic shift. To maintain intrinsic pH, the donor phase was monitored in real time with a pH dip probe and the pH was maintained at pH=12 by titrating 2N sodium hydroxide.

Positron Annihilation Lifetime Spectroscopy (PALS)

Radioactive ²²Na deposited and sealed in a thin kapton film was used as the positron source. This source was placed between two 41 mm×41 mm×1.3 mm sheets of PDMS. This configuration was found to effectively stop the majority of positrons (excluding the 8% stopped in the kapton film) in the sample PDMS. Lifetime measurements were initially taken in both air and vacuum. There was a lower event acquisition rate in the vacuum setup due to the increased distance necessary to fit the vacuum chamber between the detectors. With the ability to mathematically compensate for the pick-off annihilation, the characterization of the free volume voids was primarily run in air at and above 20° C., while the sub 20° C. was run under vacuum.

The lifetime of the particle called positronium (Ps) is most important in analyzing the pore properties of PDMS. Ps is analogous to a hydrogen atom, but with no nucleus and a positron (anti-matter electron) that orbits with an electron in a triplet state energy configuration. Since Ps can trap in open volume voids, this positronium is directly sensitive to the pore size in which it resides. The other two short lifetimes are related to singlet Ps and positrons that annihilate with an electron without forming Ps and will not be considered further. All fitting of the PALS spectra were done using a customized version of the Posfit program.³⁴

PDMS membranes were not returned into hexane to remove any non-crosslinked material nor was the cured membrane put into vacuum to attempt to remove any latent hexane, as proposed by others.³¹ However, high vacuum was used during a PALS measurement to see if there was any change in the lifetime as any hexane was “extracted” from the membrane. There was no irreversible change in positronium lifetime when the vacuum and air samples (after compensating for known air effects on the positronium life) were compared.

PALS Thermal Expansion Series

A PDMS membrane was sectioned for PALS analysis. The R_{T} positronium lifetime was measured. The same sample was then heated to a target temperature and held at that temperature until sufficient data was gathered for a positronium measurement at the target temperature. The same sample was then brought back to R_{T}, where the positronium lifetime was measured again. This cycle was repeated until all the temperature values were measured. For measurements at cryogenic temperatures, the sample was cycled between R_{T} & −230° C. with data taken at selected temperatures in between.

Error Bars

All error bars are reported as the standard error of the mean unless n<5, in which no summary statistic is given (mean, SEM).

### Example 2

Paracellular Type (Pore) Drug Transport in PDMS

Pore transport in PDMS was measured by PALS to evaluate whether pores play a significant role in the overall conduction of drug molecules from donor to receiver phases. Positron Annihilation Spectroscopy has been used for 40-50 years to characterize single vacancies and vacancy clusters. Positronium Annihilation Lifetime spectroscopy (PsALS or PALS), over the same time course, has been used to measure sub-nanometer and intermolecular voids in polymers, making this technique a robust method for probing the porous part of the PDMS polymer network. When a positron is injected into materials, it will eventually annihilate with an electron with the complete conversion of the pair's combined mass, m, into high-energy photons with total energy E=mc². There are two types of particles that are examined during PALS analysis, i.e., free positron annihilation (with electrons in the target material) and Positronium (Ps) annihilation. Both positrons and Ps seek out and localize in vacancies/voids in metals and insulators. Simple coulomb attraction forces positrons into electron-decorated vacancies in metals, whereas in insulators the reduced dielectric interaction in a void energetically favors trapping neutral Ps in low-density regions. Ps has two states, singlet (para-) and triplet (ortho-), depending on the relative spin state of the positron and electron. The self-annihilation lifetime of para-Ps is short, i.e., 125 ps, and this rapid singlet annihilation occurs with the emission of two back-to-back gamma rays of 511 keV. However, ortho-Ps (o-Ps) in vacuum is required to annihilate into at least three photons to conserve angular momentum, and this slower, triplet process has a long, characteristic lifetime of 142 ns. Lifetime spectroscopy can easily distinguish this long-lived triplet state of Ps; therefore, o-Ps plays the key role in probing porous materials.³⁴⁻³⁵

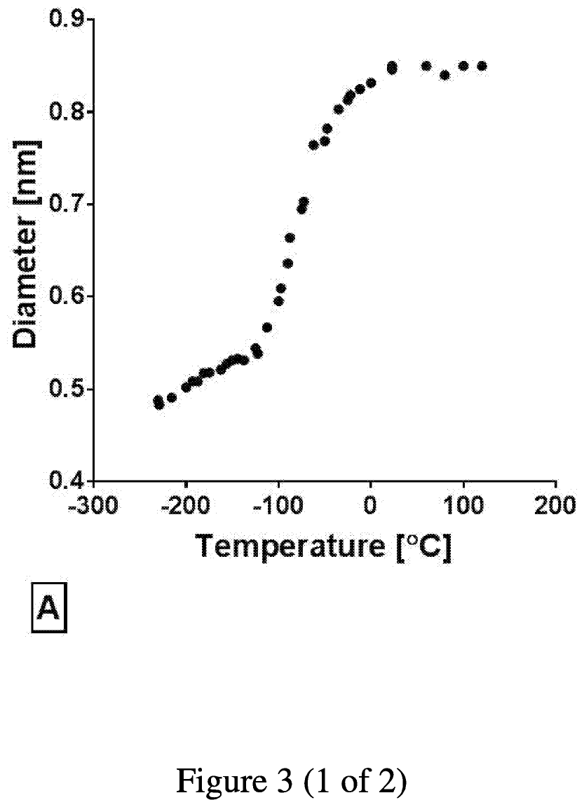

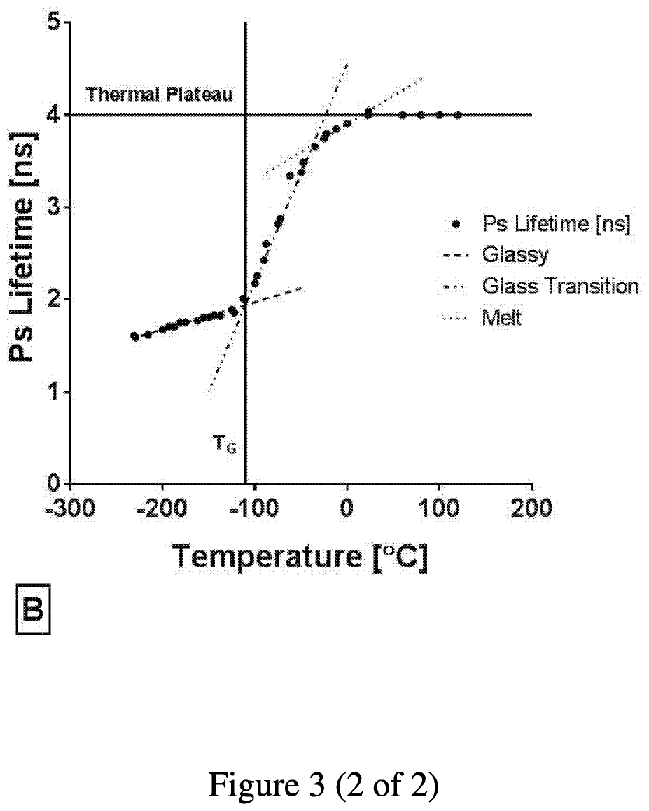

To determine whether the voids in PDMS form an interconnected porous network or are isolated voids, a “beam PALS” spectrometer, in which a low energy focused beam of positrons is used to shallowly implant positrons and form Ps close to the PDMS sample surface, was used to resolve pore connectivity.³⁴ Ps can diffuse in an interconnected porous network and escape into vacuum producing a readily distinguishable approximately 142 ns lifetime component. Using beam energies (mean positron implant depths) of 1.2 keV (40 nm), 3.2 keV (180 nm) and 4.2 keV (280 nm), the telltale 140 ns vacuum component was not found, indicating no Ps diffusion. It is conclusive that the voids of PDMS are isolated. Positronium lifetimes were converted into a spherical pore diameter over the range of interest using the Tao-Eldrup model (assuming a simple spherical pore model).^{34, 36, 37} In FIG. 3, the ability to detect very small changes in void size by positronium PALS is demonstrated by measuring the void size in the membrane at different temperatures. A consequence of being able to detect such small changes in void size is that physical changes in PDMS, such as thermal expansion transition points, can be easily detected using PALS.

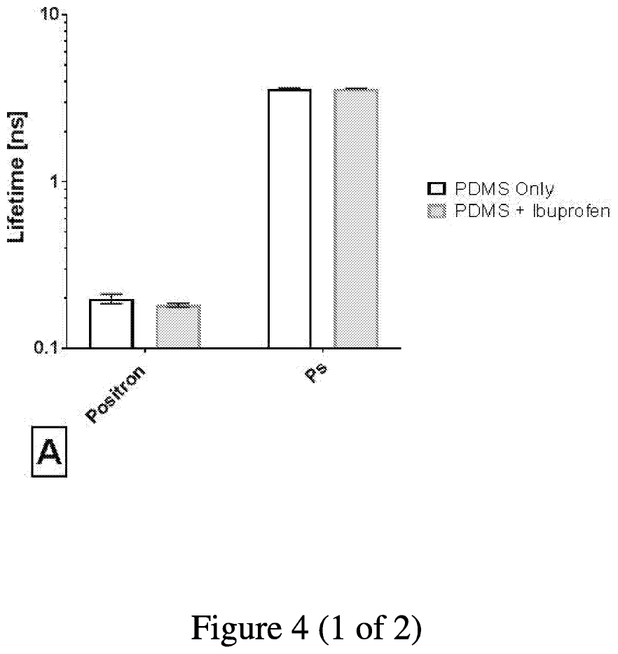

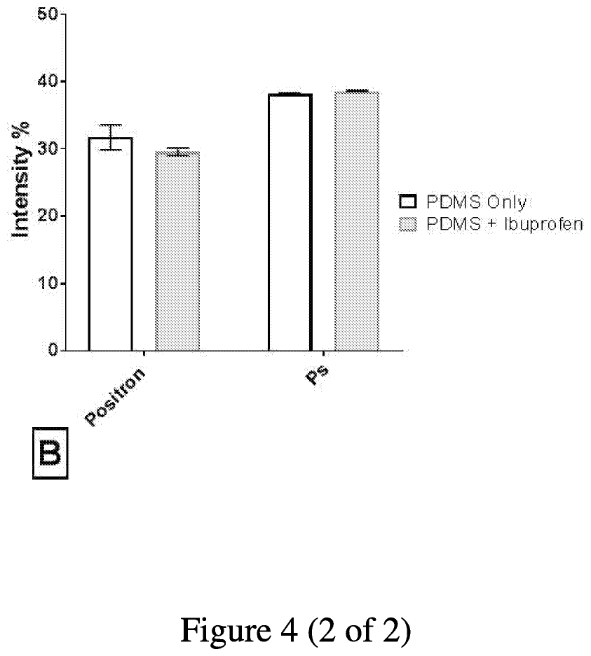

To see whether drug accumulated in the pores of the membrane, PALS was used to measure a PDMS membrane before and after equilibrium partitioning with ibuprofen (FIG. 4). Ibuprofen was chosen as a model drug because of its high lipophilicity and the ability to measure the difference in mass of drug in the membrane before and after the partitioning experiment. There was no statistical difference in the positronium lifetimes, but there was a 1.76 mg±0.12 mg (n=7) mass increase in 25% by mass curing agent PDMS membranes, and a 2.90 mg±0.38 mg (n=6) mass increase in 3.2% by mass curing agent PDMS membranes. This mass increase agrees with the measured partition coefficient for ibuprofen in PDMS.

### Example 3

Transcellular Type (Bulk) Drug Transport in PDMS

Partitioning of Molecular Probes into PDMS

Bulk transport properties of PDMS were measured to evaluate PDMS fitness for the mimicking human oral absorption. The partition coefficient was calculated according to Equation 4.

$$K = \frac{{c}_{mem}^{*}}{{c}_{aq }^{*}} \qquad ( 4 )$$

### Where:

K=Partition coefficient
Cₘₑₘ*=Equilibrium concentration of drug in membrane

C_{aq}*=Equilibrium concentration of drug in the aqueous media

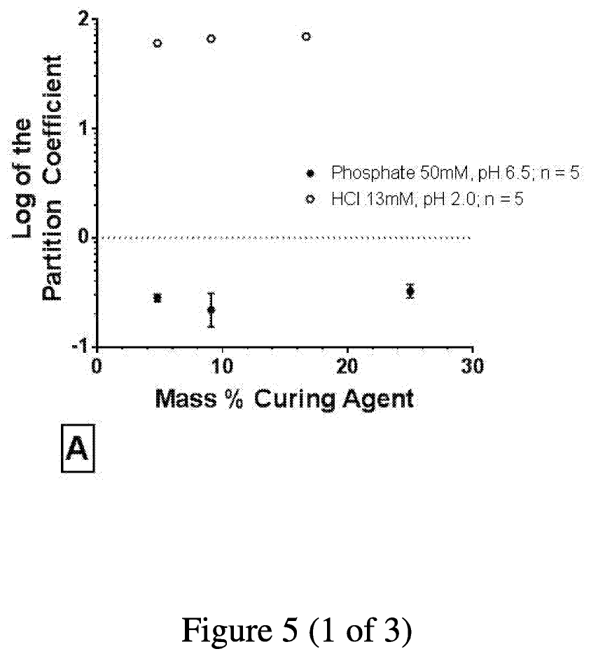

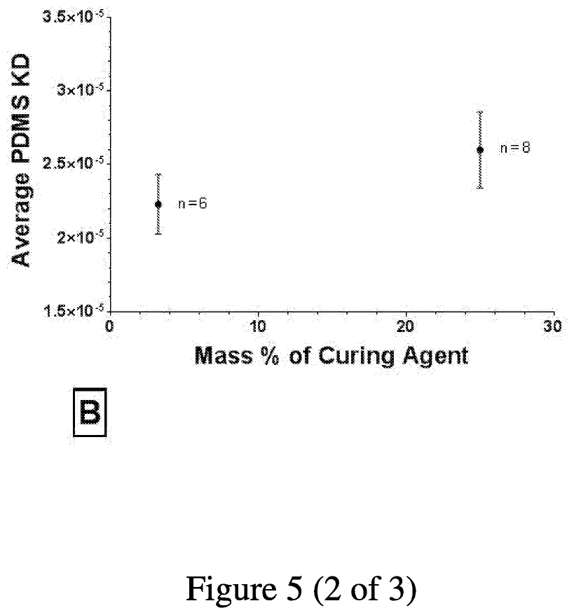

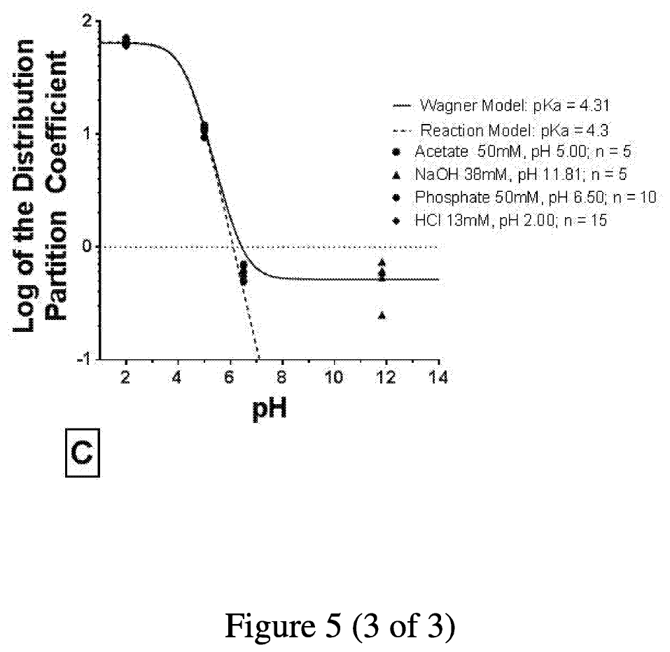

The first study conducted was to determine if process variations in fabrication would lead to differences in the equilibrium partition of the model drug, ibuprofen (FIG. 5A). Four different mass % of curing agents (4.9%, 9.1%, 16%, and 25%) were examined at two different pH conditions (2.00 HCl 13 mM & 6.50 phosphate 50 mM). The Log D_{PDMS} was measured as a function of pH at pH 2.00, 5.00, 6.45, and 12.00 using 13 mM HCl, 50 mM acetate, 50 mM phosphate, and 39 mM NaOH buffers, respectively, at R_{T} (FIG. 5C).

The Log D relationship is given in Equation 5 for a monoprotic acid (model 1) (see Supporting Information for derivation).³⁹ In Table 3 the Log D was also calculated using the Wagner model which accounts for ionized drug partitioning (Equation 6, model 2) and the pKa of ibuprofen was back calculated to confirm the validity of the fit for both models (for model 2, Equation 8 was used to transform the shifted pKa from Equation 6 back to the true pKa).⁴⁰

$$\begin{matrix}Model 1 \text{:} & \\ log D = log K - log ( 1 {0}^{p H - p K a} + 1 ) & ( 5 ) \\ Model 2 \text{:} & \\ log D = log ( {P}_{u} {f}_{E} ) = log [ {P}_{u} \frac{{f}_{u} {P}_{u} + ( 1 - {f}_{u} ) {P}_{i}}{1 + {f}_{u} {P}_{u} + ( 1 - {f}_{u} ) {P}_{i}} ] & ( 6 ) \\ {f}_{u} = \frac{1}{1 + 1 {0}^{p H - p K a}} & ( 7 ) \\ p {H}_{from fit} = pK {a}_{a c t u a l} - log ( {P}_{u} - 1 ) & ( 8 )\end{matrix}$$

### Where:

log D=the log₁₀ of the distribution coefficient
log K=the log₁₀ of the intrinsic non-ionized partition coefficient
Pᵤ=the intrinsic non-ionized partition coefficient
Pᵢ=the intrinsic ionized partition coefficient
f_{E}=fraction extracted by the membrane
fᵤ=fraction of non-ionized drug in solution
PH_{from fit}=input pKa in the Log D equation
pKa_{actual}=the un-shifted pKa from fit, the true pKa of the molecule

TABLE 3
Comparing the reaction model and the Wagner model, for
estimating the distribution of partition coefficients as a function
of pH, also used to predict the pKa of Ibuprofen using PDMS.

| IBUPROFEN PKA | | | MEASURED PDMS | | | |
|---|---|---|---|---|---|---|
| LIT. | | | LOGP_{U} | LOGP_{I}^{I} | | |
| PKA | MODEL | MODEL | ± | ± | | |
| VALUE | 1 | 2 | SE | SE | P_{U} ± SE | P_{I EQ. 6}^{II} |
| 4.4⁶ | 4.3 | 4.31 | 1.81 ± | −0.286 ± | 65 ± | 0.008 |
| | | | 0.01 | 0.080 | 1.05 | |
| | | | N = 15 | N = 15 | N = 15 | |

^{I}experimentally determined at pH 11.81
^{II}using the experimental data at pH 11.81 and fᵤ~0, Pᵢ was calculated to fit equation 6

Permeability is a function of the partition coefficient (Equation 9).

$$P = \frac{K D}{h} \qquad ( 9 )$$

### Where:

K=partition coefficient (unitless)
D=diffusion coefficient [cm² s⁻¹]
h=thickness of the membrane [cm]

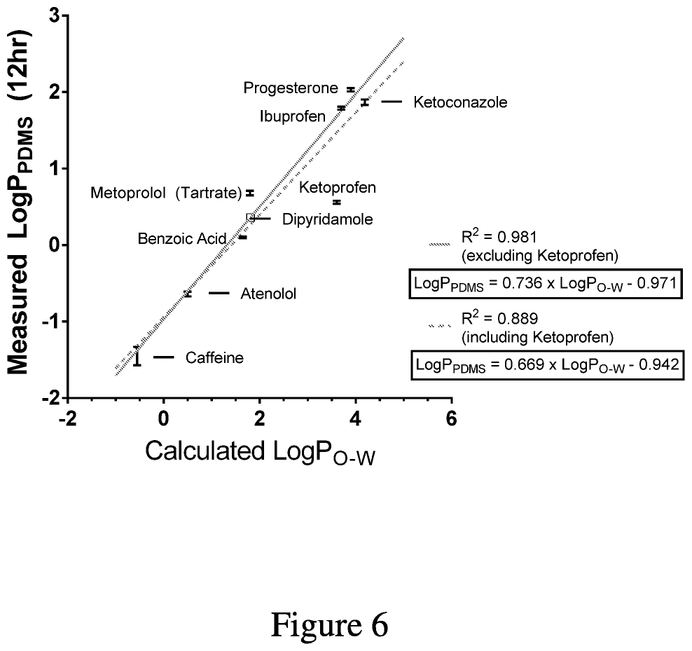

Therefore, it was important to see if the partition coefficient could be predicted for any drug in PDMS. A simple correlation was created between the predicted partition coefficient of drugs in the octanol-water system (the standard reference system for partitioning) and the PDMS system. FIG. 6 indicates that a linear correlation can be used to translate the well-established octanol-water partition coefficients to the PDMS system. Ketoprofen consistently deviated from the expected lipophilicity correlation. In vivo, ketoprofen has a large human GI permeability, even though its pKa (3.89) is lower than ibuprofen (4.4).^{6,41} Based on ketoprofen's pKa alone, it would be expected that it would be more ionized in the human GI tract than ibuprofen, and thus not absorbed as well. This indicates that ketoprofen's lipophilicity is high enough to drive the drug across the human GI membrane, as explained by Wagner et al.⁴⁰ However, it is possible that the deviation in PDMS partitioning comes from ketoprofen's high polar surface area. Due to the methyl groups that decorate the backbone of PDMS, the polar-non-polar interaction at the solid-liquid interface would be stronger for molecules that have a more polar surface area.

Diffusion of Molecular Probes through PDMS Membranes

Membrane permeability was calculated from the linear slope of the concentration versus time curve for each experiment. This pseudo-steady-state linear region was determined by calculating the linear regression coefficient of the slope and optimizing the range to achieve a R² as close to 1 as possible. The slope of this line, when multiplied with the receiver volume, gave the mass transfer coefficient (Equation 10).

\dot{m}=m_{pseudo-steady}×V_{receiver} (10)

### Where:

\dot{m}=mass transfer coefficient [μg s−1]
m_{pseudo-steady}=slope of concentration versus time plot in the pseudo-steady state region [μg mL-1 s−1]
V_{receiver}=volume of the drug receiving phase [mL]

The effective permeability was calculated using permeability layer theory (Equation 11) and, as a check, diffusion coefficient was calculated using Crank's uniform initial distribution and surface concentration different for diffusion in a plane sheet (Equation 9, 12-13).⁴² Permeability layer theory considers every layer in the diffusion system (solid and liquid interfaces), while Crank's approach examines diffusion only within the membrane itself.

$$\begin{matrix}\frac{1}{{P}_{e f f}} = \frac{{A}_{membrane} ( S \times n )}{\dot{m}} & ( 11 ) \\ QT = \frac{D {C}_{2}}{h} ( t - \frac{{( h )}^{2}}{6 D} ) & ( 12 ) \\ D = \frac{{Q}_{{T}^{h}}}{{C}_{2}} & ( 13 )\end{matrix}$$

### Where:

P_{eff}=permeability of molecule through the membrane & aqueous boundaries [cm s⁻¹]
A_{membrane}=area of membrane available to transport [cm²]
S=the solubility of the molecule [μg mL⁻¹]
\dot{m}=mass transfer coefficient [μg s⁻¹]
Q_{T}=mass per unit Area [μg cm⁻2] (from the slope of the receiver concentration profile multiplied by V_{aq}/A_{membrane}
D=the diffusion coefficient of the molecule through the membrane [cm² s⁻¹]
C₂=concentration of the molecule at the inner surface of the membrane (C_{aq total}*K) [μg cm⁻³]
t=time [s]
and the membrane permeability from permeability layer theory is Equation 14 and Crank's membrane permeability is Equation 15 (both derived in Example 11).

$$\begin{matrix}{[ \frac{{A}_{m e m b r a n e} \times S}{m \times {V}_{recei νer}} - \frac{{h}_{a q}}{{D}_{a q}} ]}^{- 1} = {P}_{P D M S} & ( 14 ) \\ \frac{\frac{{m}_{dC / {dt}^{\times V} receiver}}{{A}_{m e m b r a n e}}}{{C}_{2}} = ( \frac{{Q}_{T}}{K \times {C}_{a q}} ) = {P}_{P D M S} & ( 15 )\end{matrix}$$

### Where:

h_{aq}=Levich boundary layer thickness [cm]
D_{aq}=drug's aqueous diffusion coefficient [cm² s⁻¹]
m_{dC/dt}=slope of the concentration versus time curve in the receiver compartment [μg cm⁻³ s⁻¹]
P_{PDMS}=the permeability of the PDMS membrane [cm s⁻¹]

The difference in the permeability when the aqueous boundary permeation is assumed to be of negligible importance, and when the contribution is accounted for, was examined. For drugs with a Log K_{PDMS}<1.5, the permeability difference is <10% and for higher partitioning drugs (1.5<Log K_{PDMS}<2.1), results were found to have up to a 25% difference. The lag time (time to steady-state transport) was calculated by solving the pseudo-steady-state linear regression equation for Y=0, where Y=concentration and X=time. This lag time can be predicted by Equation 16.⁴²⁴³

$${t}_{p s e u d o - s . s .} = \frac{{h}_{m e m b r a n e}^{2}}{6 D} \qquad ( 16 )$$

### Where:

t_{pseudo-s.s.}=time to pseudo steady state transport [s]
h_{membrane}=membrane thickness [cm]
D=diffusion coefficient of the molecule through the membrane [cm² s⁻¹]

It was hypothesized that modulating the elastic modulus of PDMS could modulate the drug permeability. FIG. 5B and Table 4 show the difference in permeability when the curing agent and, by association, Elastic modulus, is varied. There is a statistical difference in permeability between membranes with 3.2% and 25% by mass curing agent, but in practice the difference is of negligible importance. This is most likely because PDMS is in the viscous flow regime at 37° C., making it relatively easy for backbone chain movement to accommodate diffusing species. The permeation data is presented as the K×D product (P×h). This eliminates experimental variability from the membrane thickness, and allows for more accurate comparisons of permeability with the added benefit of permeation prediction at any membrane thickness. Additionally, the apparent diffusion coefficient through the silastic membranes, such as PDMS, is known to be the product of the intrinsic diffusion coefficient of the drug through the polymer network and the apparent partition coefficient of the uncharged species between the solvent and the membrane. Knowledge of the partition coefficient should permit prediction of the relative diffusion through PDMS membranes.³³ So, the K×D product can be thought of as both the diffusivity of a molecule through PDMS and a normalized-by-thickness permeability.

TABLE 4
The tabulated results of the two-tailed t-test to determine if the null hypothesis (no
difference in the permeation between the 3.2% and 25% w/w curing agent membranes) was
valid. The null hypothesis is rejected based on the t-statistic, however the practical
difference in permeation is negligible for the intended application of ivR absorption.

| | | TOTAL | | |
|---|---|---|---|---|
| | AVERAGE | STANDARD | TWO TAIL | |
| COMPOSITION | K × D | ERROR | T-TEST | P -VALUE |
| [MASS %] | [×10⁻⁵CM² S⁻¹] | [×10⁻⁶CM² S⁻¹] | STATISTIC | 0.0103 |
| 25 | 2.60 | 2.58 | −3.04 | DF = 12 |
| | N = 8 | N = 8 | | (11.9) |
| 3.2 | 2.23 | 2.03 | | 98.97% |
| | N = 6 | N = 6 | | CONFIDENCE TO |
| | | | | REJECT NULL |
| | | | | HYPOTHESIS |

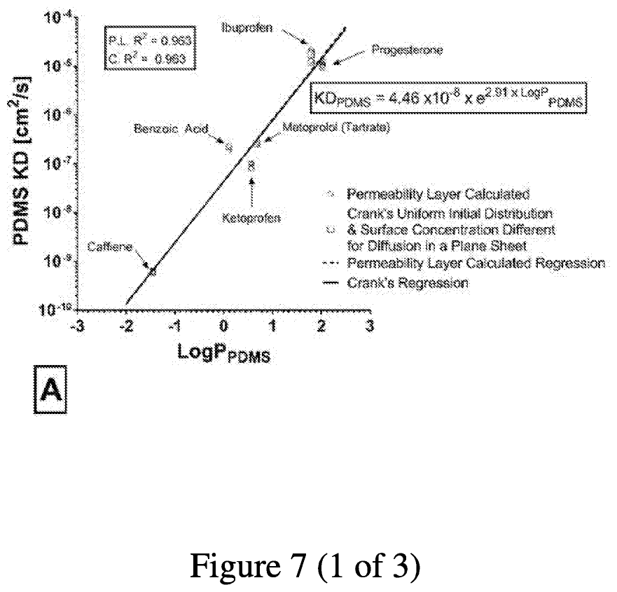

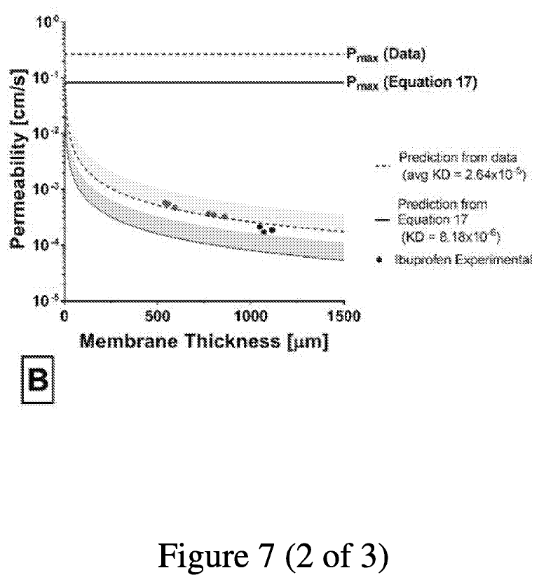

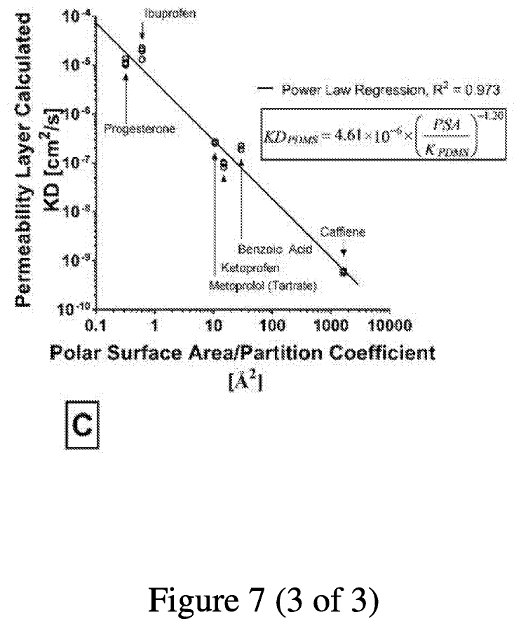

The K×D product was measured for the same set of drugs for which the partition coefficient was measured (FIG. 7A and Equation 17).

$$\begin{matrix}K \times {D}_{P D M S} = 4.46 \times 1 {0}^{- 8} \times {e}^{2.91 \times L o g {P}_{P D M S}} & ( 17 ) \\ K \times {D}_{P D M S} = 4.61 \times 1 {0}^{- 6} \times {( \frac{P S A}{{K}_{P D M S}} )}^{- 1.20} & ( 18 )\end{matrix}$$

The permeation of model drug, ibuprofen, behaved according to Equation 12, which demonstrates that permeability can be predicted over a wide range membrane thicknesses, as seen in FIG. 7B. The deviation of ketoprofen in the octanol-water partition coefficient to PDMS partition coefficient is not observed in the PDMS permeability versus PDMS partition coefficient correlation. Therefore it possible that ketoprofen has a lower affinity for PDMS than octanol due its high polar surface area relative to its partition coefficient (FIG. 7C and Equation 18).

In each permeation experiment, the donor-phase-containing drug was at a non-ionizing pH if the drug was ionizable and the receiver phase was at a completely ionizing (>99%) pH. The values of the non-ionized thermodynamic solubility at 37° C. that were used in the permeation calculations were reported in Table 5. Progesterone solubility was determined twice, the second time as an analytical check. This check shows that the method used to determine solubility was unaffected by the amount of dilution used to obtain the solubility (diluted 3.5× and 10.5×, respectively).

TABLE 5
For each rotating diffusion cell experiment, the donor side
concentration was about 3-fold greater than the solubility
reported from drugbank.com. The actual solubilities were
the measured for each molecule in the donor-solution conditions
present during the permeation experiments. These solubility
values were used in all permeation calculations.

| | | NON- | |
|---|---|---|---|
| | | IONIZED | STANDARD |
| | DILUTION | SOLUBILITY | ERROR |
| DRUG | FACTOR | [MG ML⁻¹] | [MG ML⁻¹] |
| IBUPROFEN | N = 5 | 60 | 1.18 |
| | REP = 2 | I | |
| KETOPROFEN | N = 5 | 147 | 2.3 |
| | REP = 2 | I | |
| BENZOIC ACID | N = 5 | 69.8 × 10² | 2.31 × 10² |
| | REP = 2 | I | |
| CAFFEINE | N = 3 | 47.0 × 10³ | 0.332 × 10³ |
| | REP = 2 | II | |
| PROGESTERONE | N = 4 | 21.3 | 0.26 |
| | REP = 2 | I | |
| PROGESTERONE | N = 4 | 20.9 | 0.21 |
| (ANALY. COMP.) | REP = 2 | I | |

I = pH 2.00 13 mM hydrochloric acid buffer at 37° C.
II = pH 6.45 50 mM phosphate buffer at 37° C.
N = number of individual samples
Rep = number of times replicated

Table 6 shows the experimentally measured permeability, diffusion coefficient, and lag time values for each drug, along with experimental conditions used to generate those values. These permeability measurements are in the intrinsic ionization state (completely non-ionized), but it is expected that ivR testing will occur at pH values where many drugs will have some fraction of ionized molecules. The Wagner models and Winne models for pH-dependent absorption show that significant absorption occurs in vivo even under pH conditions were there is a large fraction of ionized drug.^{40, 44} From a characterization standpoint, however, the use of the relationship between Log K_{PDMS} and PDMS permeability is valid for any Log D_{PDMS}. An experimental compound may be partially ionized under physiologic pH conditions, but if the Log D_{PDMS} can be measured or predicted, so can the correct PDMS permeability. PDMS reflects the in vivo situation, where ionized drug is present in the absorption pathway and absorption rate will be a function of pH.

TABLE 6
The tabulated results of the rotating diffusion cell experiments. The average membrane thickness, average PDMS permeability,
and average PDMS KD are calculated, as described herein. The partition-independent diffusion coefficient was calculated by dividing
the average PDMS KD by the 10^{LogK} for PDMS. To compare the diffusivities in the polymer with those in the aqueous environment the
Hayduk-Laudie diffusion coefficient was calculated.⁴⁵ Lag times were measured using a linear regression from the concentration versus
time profile and represent the time to steady state transport across the membrane. The predicted lag times from Crank′s method (Equation 16).

| | | AVG. | | | PARTITION | AQUEOUS | | | | |
|---|---|---|---|---|---|---|---|---|---|---|
| | | MEM- | AVG. | AVG. | INDEPENDENT | HAYDUK- | | LAG | LAG | |
| DRUG | DONOR/ RECEIVER BUFFER | BRANE THICK. [MM] | PDMS PERM. [CM S⁻¹] | PDMS (K*D) [CM² S⁻¹] | DIFF. COEFF (K*D)/10^{LOGK}^{_{PDMS}} [CM² S⁻¹] | LAUDIE DIFF. COEFF. [CM² S⁻¹] | PDMS LOGK | TIME (EXP.) [MIN] | TIME (PRED.) [MIN] | $\frac{{t}_{{Lag}_{pred .}}}{{t}_{{Lag}_{exp .}}}$ |
| IBUPROFEN | I/III | 651 | 2.82 × 10⁻⁴ | 1.83 × 10⁻⁵ | 2.99 × 10⁻⁷ | 8.41 × 10⁻⁶ | 1.79 | 68 | 158 | 2.6 |
| | | N = 3 | N = 3 | N = 3 | N = 3 | | N = 5 | N = 3 | N = 3 | N = 3 |
| PROGESTERONE | I/III | 662 | 1.73 × 10⁻⁴ | 1.14 × 10⁻⁵ | 1.07 × 10⁻⁷ | 6.72 × 10⁻⁶ | 2.03 | 115 | 158 | 1.4 |
| | | N = 4 | N = 4 | N = 4 | N = 4 | | N = 5 | N = 4 | N = 4 | N = 4 |
| BENZOIC ACID | I/III | 676 | 3.10 × 10⁻⁶ | 2.08 × 10⁻⁷ | 1.66 × 10⁻⁷ | 1.28 × 10⁻⁵ | 0.10 | 25 | 238 | 9.3 |
| | | N = 2 | N = 2 | N = 2 | N = 2 | | N = 5 | N = 2 | N = 2 | N = 2 |
| KETOPROFEN | I/III | 653 | 1.45 × 10⁻⁶ | 9.46 × 10⁻⁸ | 2.58 × 10⁻⁸ | 8.02 × 10⁻⁶ | 0.56 | 232 | 482 | 2.8 |
| | | N = 3 | N = 3 | N = 3 | N = 3 | | N = 5 | N = 3 | N = 3 | N = 3 |
| METOPROLOL | I/III | 502 | 5.25 × 10⁻⁶ | 2.64 × 10⁻⁷ | 5.46 × 10⁻⁸ | 7.20 × 10⁻⁶ | 0.68 | 161 | 386 | 2.4 |
| TARTRATE | | N = 2 | N = 2 | N = 2 | N = 2 | | N = 5 | N = 2 | N = 2 | N = 2 |
| CAFFEINE | I/III | 703 | 8.59 × 10⁻⁹ | 6.02 × 10⁻¹⁰ | 1.70 × 10⁻⁸ | 9.63 × 10⁻⁶ | −1.45 | 115 | 571 | 5.1 |
| | | N = 3 | N = 3 | N = 3 | N = 3 | | N = 5 | N = 3 | N = 3 | N = 3 |

I = pH 2.00 13 mM hydrochloric acid buffer at 37° C.
II = pH 12.00 39 mM sodium hydroxide buffer at 37° C.
III = pH 6.45 50 mM phosphate buffer at 37° C.
IV = pH 8.00 50 mM phosphate buffer at 37° C.

Mechanical Properties and Microstructural Analysis

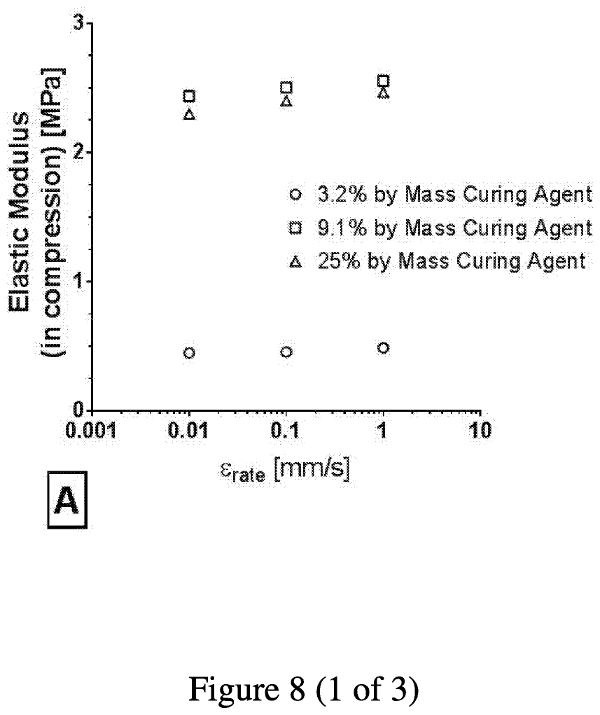

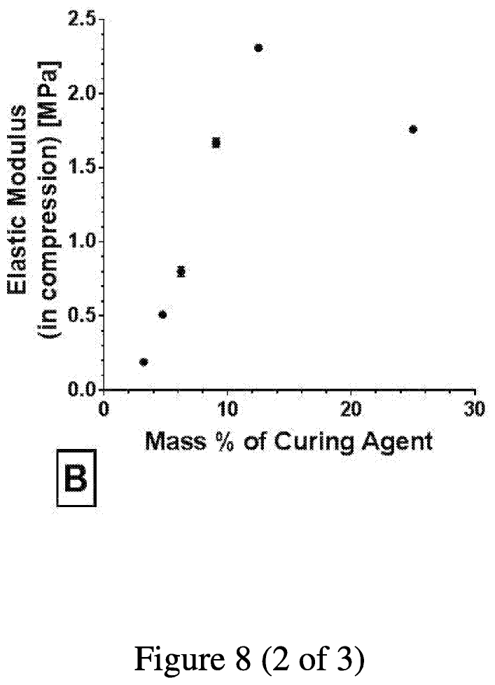

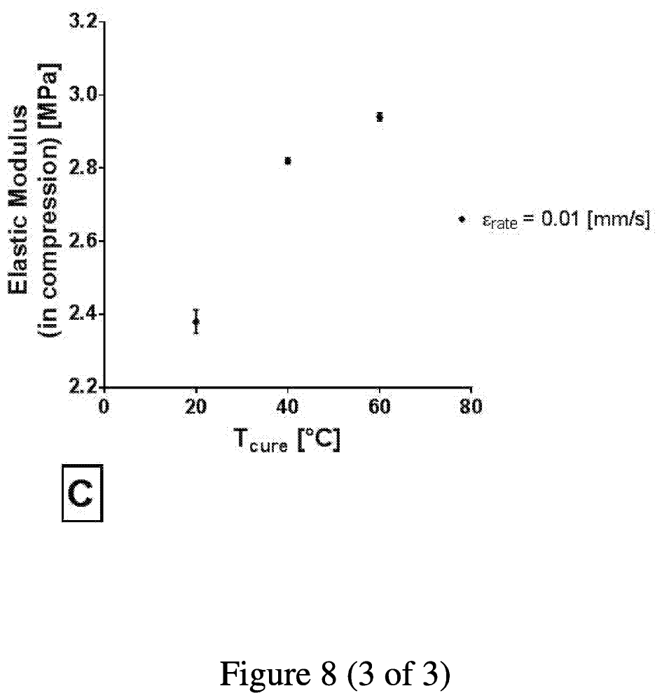

The stiffness of the polymer (crosslinking) was expected to govern the transport of drug molecules. Additionally, modulating the stiffness of the network was expected to provide a secondary method of modulating the permeability of PDMS membranes. It was also necessary to understand the material's mechanical properties for fabrication of an in vitro absorption material, such as a membrane, e.g., an ultra-thin membrane. Before any mechanical testing was conducted, a study of the strain rate effect was conducted. The initial studies were performed in tension, but the material was found to be too soft for use in the testing cell. Therefore, all mechanical measurements disclosed herein were completed in compression. FIG. 8A shows there is a minimal effect of strain rate on the elastic modulus of PDMS and, therefore, the smallest strain rate was used for the remainder of mechanical testing (0.01 mm/s). Data consistent with the foregoing expectations was disclosed in reference⁴⁶, and the elastic modulus versus mass percent of curing agent experiment was recreated in FIG. 8B. This relationship was measured to examine the difference in the elastic modulus between hexane-solubilized, drop-cast PDMS produced as disclosed herein and PDMS created by simply mixing the two parts of the Sylgard 184 kit.⁴⁶ The PDMS produced by hexane homogenization was less stiff across all concentrations of curing agent as compared to the non-hexane method. Additionally, in the hexane casting method, there is an apparent critical point beyond which the addition of curing agent no longer enhanced the elastic modulus. This was not demonstrated in the non-hexane method in reference⁴⁶. Finally, the effect of curing temperature was measured to see if the elastic modulus could be altered during curing. FIG. 8C indicates that a small increase in the elastic modulus can be achieved by curing at temperatures above R_{T}.

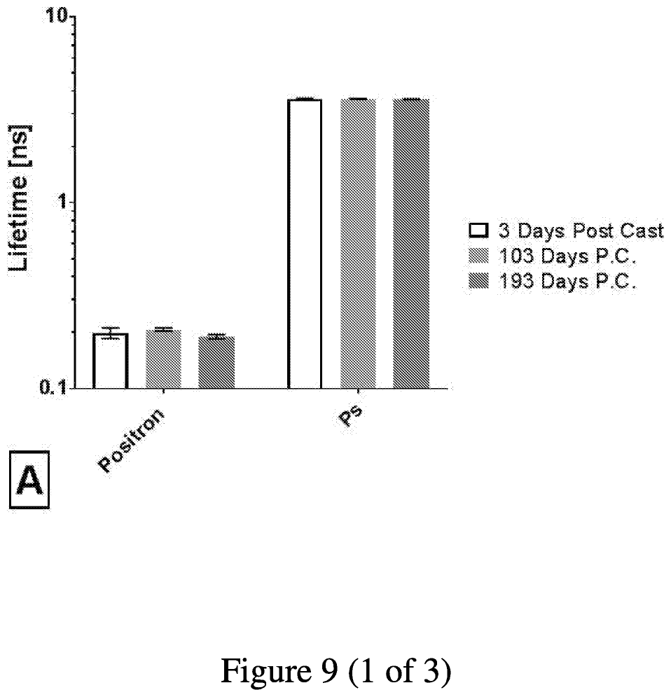

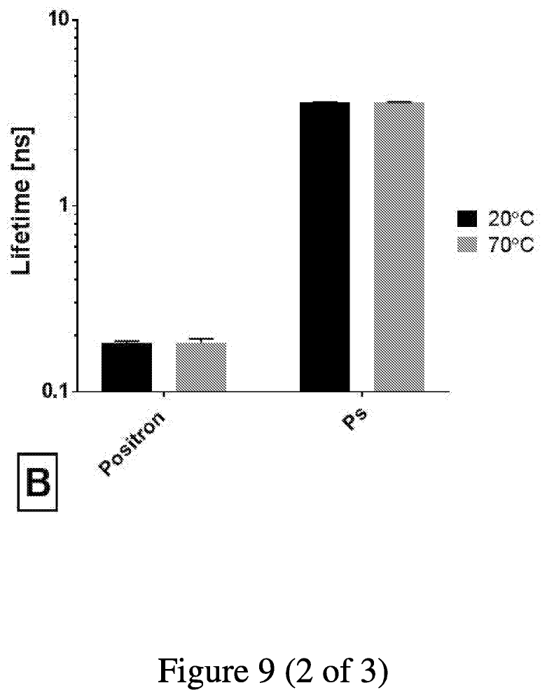

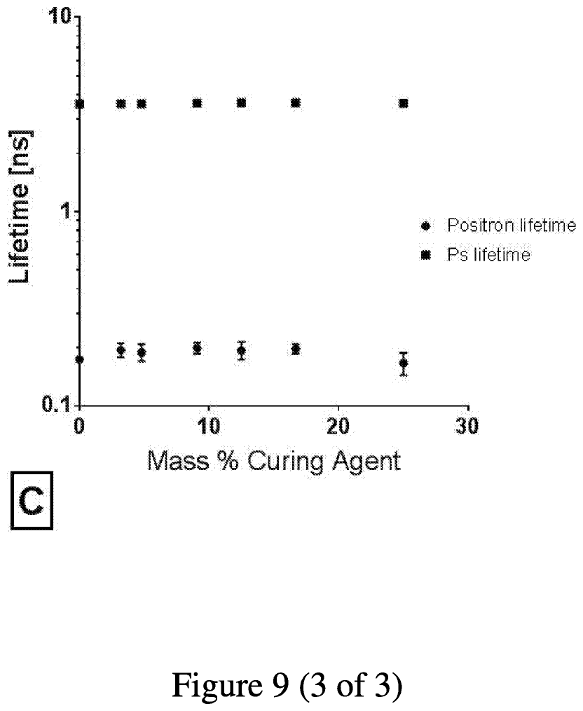

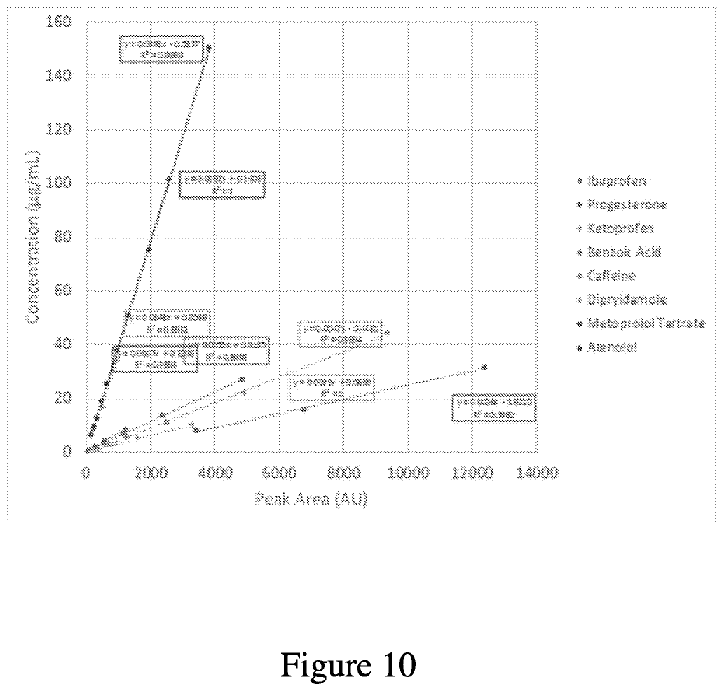

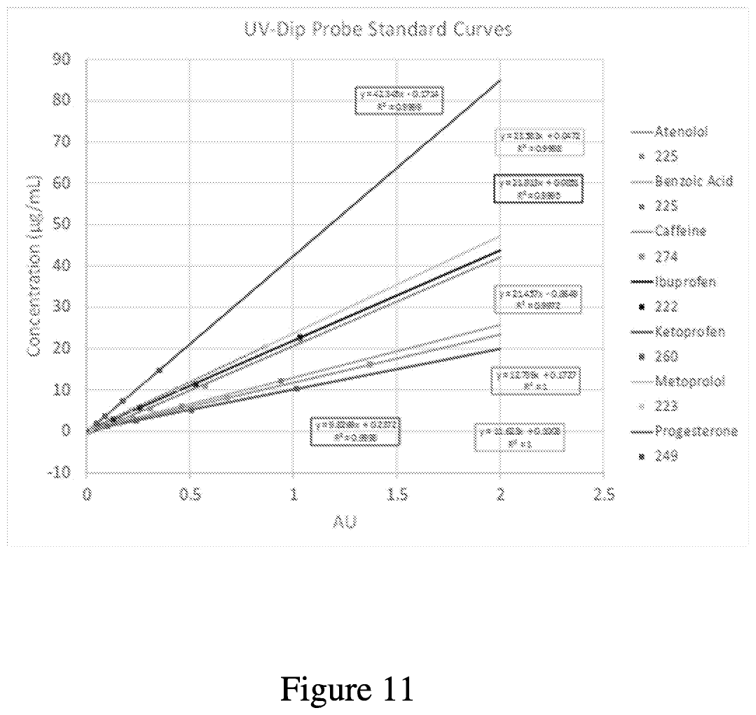

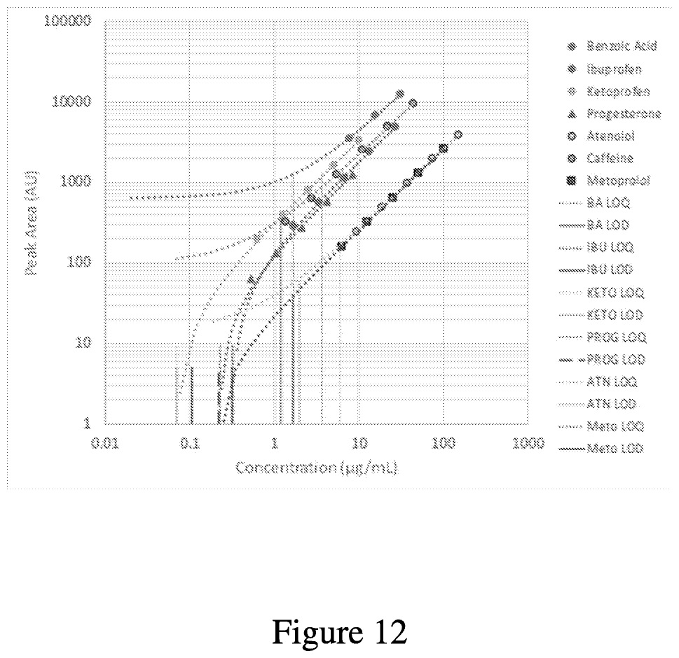

To evaluate R_{T} pore stability, the PDMS void structure was quantified via PALs at 3, 103, and 193 days after casting (FIG. 9A), and no statistical change was observed in the positronium lifetime over this time course. For accelerated casting procedures (above 20-25° C. and faster than 3-7 days), PDMS membranes were cured at 70° C. to determine the effect elevated curing temperature had on the final R_{T} void structure. There was no change in the positronium lifetime between 20° C. and 70° C. cure temperatures (FIG. 9B). 70° C. was chosen as the upper limit because that was the temperature at which hexane boils. Boiling hexane produces bubbles within the final cured membrane that were potentially undesirable. Membranes were cured at 60° C. without significant bubbles forming. Finally, PALS was used to measure if a change occurred in the void structure due to process variation (FIG. 9C). The amount of curing agent was varied from 0% (pure base material) to 25% curing agent by mass. There was no statistical difference in the positronium lifetime in any composition measured.

### Example 4

Additional Materials

The materials and equipment used in the following experiments included Agilent 1100 high-performance liquid chromatography (HPLC), Extend C 18 column (3.5 μm×4.6 μm×150 mm), acetonitrile (EMD Millipore, HPLC grade), deionized water (Milli-Q purified), trifluoroacetic acid (Fisher Scientific, Optima grade), methanol (Fisher Scientific, HPLC grade), Crospovidone (UM2012-085 Lot # K-H09074), Croscarmellose Sodium Non-GMP (Material #10127157), HPMC-AS, LF (UM 2012-091 Lot #007), Microcrystalline Cellulose (PH102 UM2012-004), Mannitol, NF (Glaxo-Smith Klein UM2009-010), Dibasic Calcium Phosphate (JRS Lot #2059X UM2011-049), Lactose Monohydrate-310-NF (UM2001-018), Magnesium Stearate (UM2009-013), Sodium Dodecyl Sulfate for Electrophoresis 99% (Sigma-Aldrich), Citric Acid Anhydrous (Fisher A940-500), Ibuprofen (Albemarle Lot No. 2050-0032F), hydrochloric acid buffer pH 2.0 (USP guideline), phosphate buffer pH 6.5 (USP guideline), sodium hydroxide buffer pH 12 (NaOH+KCl), 1-Octanol, 99% pure (Acros Organics Lot # A0358670 CAS 111-87-5), polyvinylalcohol MW=25 K, 88% hydrolyzed (Poly Sciences, Inc. #02975 Lot #652279), poly(dimethylsiloxane), Sylgard 184 elastomer kit (Dow Corning), hexane (Fisher, reagent grade), Model WS-650MZ-23NPPB spin processor (Laurell Technologies), vacuum drying oven (Yamato ADP300C), 100 mm Test Grade Silicon Wafers with native silicon oxide layer (Encompass Distribution Services), Stratasys J750 printer build size: 490×390×200 mm, VEROCLEAR RGD810 (material for J750), Dimension Elite 3-D printer build size 203×203 mm, and a Tescan MIRA3 FEG Scanning Electron Microscope.

### Example 5

Design, Simulation, & Evaluation of the UTLAM Diffusion Cell

Design—Dissolution Bowl

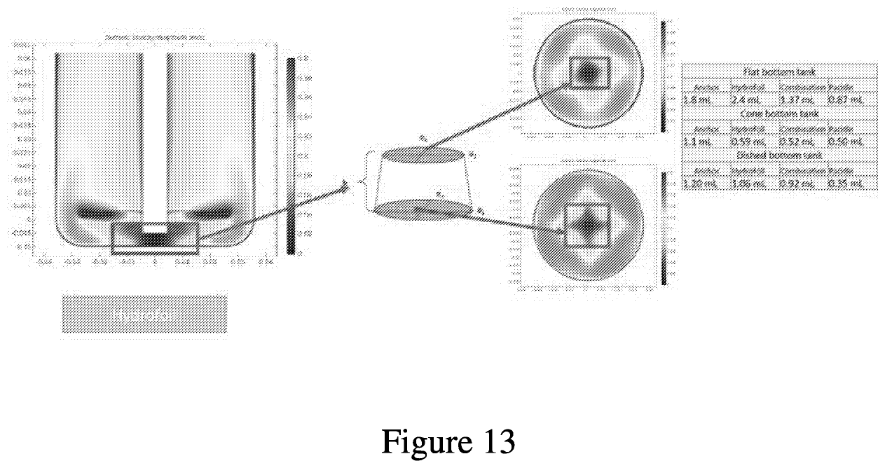

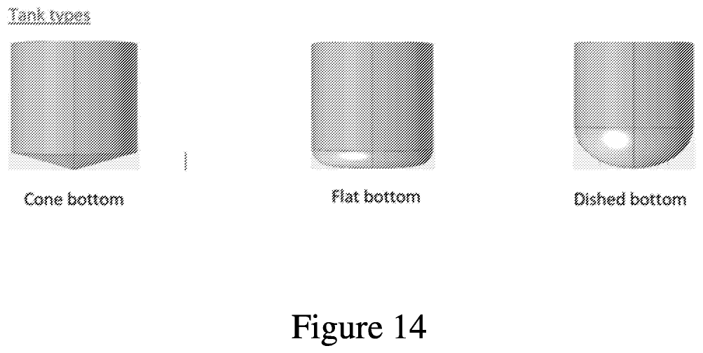

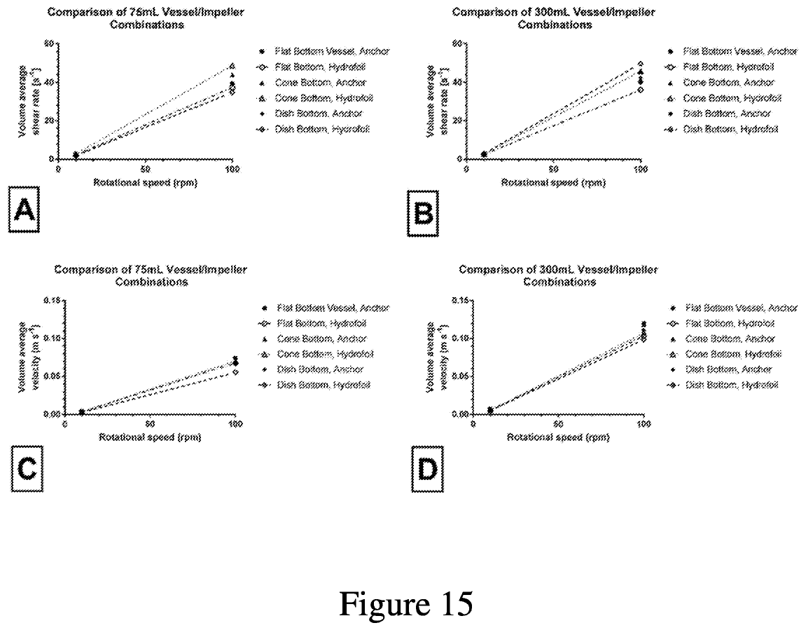

Historically, dissolution apparatuses have suffered from fluid and particle distribution heterogeneity. The standard round-bottom USP 2 vessel has been modeled in the past and has been shown to have large volumes of low or no velocity and shear, coupled with areas of intense velocity & shear. The advantage of the round-bottom vessel is that the dosage form can reproducibly sit in the same spot in the vessel and experience consistent hydrodynamic forces across many studies, however, it has been reported that dosage forms do not sit at the apex position. The weakness of this built-in feature is that the USP 2 round-bottom vessel suffers from a large dead volume immediately below the impeller, and this is where the tablet sits.⁸⁰⁻⁸⁴ During disintegration and dissolution of the solid dosage form, disintegrated particles also accumulate in this dead volume, known as “coning”. Decreasing the dead volume and increasing the homogeneity of the fluid flow to reduce the “coning” problem became the primary concerns when developing the dissolution component of the UTLAM diffusion cell. For the dissolution vessel part of the UTLAM, flat-bottom, round-bottom, and cone-shaped vessel geometries were considered (FIG. 14). The dead zone created under the shaft in each tank design was estimated, and the dead-zone volume in all designs was approximately the same (FIG. 13). For convenience and compatibility with a circular planar membrane, the flat bottom vessel configuration was chosen, which allows for a simple interface between the bottom of the dissolution vessel and the membrane to be established. Curvature around the lower edge of the dissolution bowl was maintained to prevent accumulation of particles in a sharp corner at the bottom of the vessel. An asymmetric configuration (h_{cylinder}≠D_{cylinder}) was chosen for the cylindrical flat bottom vessel to maximize the area for membrane absorption at the bottom of the vessel, while maintaining low fluid volumes. The final fabricated design has a radius of curvature of 2 mm with an impeller gap distance of 7 mm. FIG. 15 shows that the volume average fluid shear rate and velocity are independent of the shape of the tank in Algebraic Plus RANS Turbulent model CFD simulations with COMSOL.

Impeller

The UTLAM dissolution bowl and impeller were designed to balance the need for analytical robustness while maintaining physiologic hydrodynamic conditions. One unique aspect of this approach was to consider the two components pieces (vessel and impeller) together when designing, rather than considering each component as a separate entity, as has been the approach in the past. Since the UTLAM was used to disintegrate and dissolve intact drug products, the impeller's main function was to keep drug particles suspended homogeneously and to keep fluid homogeneously distributed within the dissolution vessel while maintaining lower bulk fluid shear rates. Two types of impellers were investigated using the COMSOL, the hydrofoil and the anchor, to see which impeller produced low shear and sufficient velocity profiles in the dissolution bowl while maintaining particle suspension and a homogeneous distribution of particles.

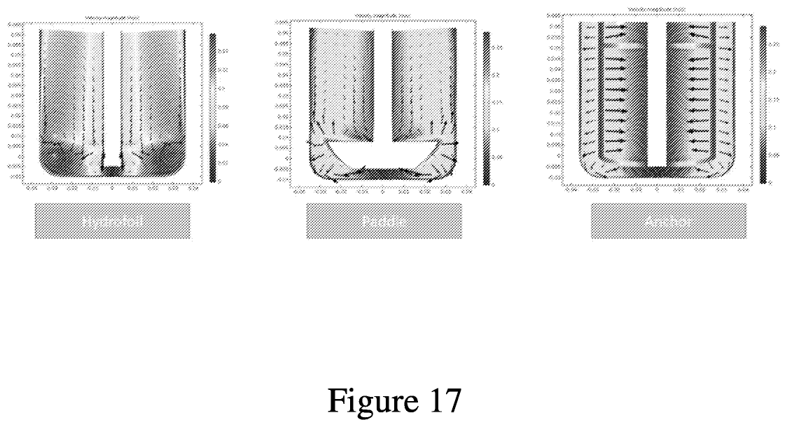

A comparison of the flow fields between two candidate impeller configurations and a traditional USP 2 paddle is shown in FIG. 17. The velocity flow fields showed that the hydrofoil provided axial (vertical flow parallel to the shaft) mixing, while the anchor provided purely radial (perpendicular to the shaft) mixing. The advantage of axial mixing is that this flow pattern promotes particle resuspension. Hydrofoils have been reported in the literature to be ideal impeller for suspending mass for mass transfer operations and was selected as the primary design for the ivR impeller.⁸⁵⁻⁸⁷

Parameter Study of the Impeller

The main criterion for the impeller is to generate physiologic hydrodynamic conditions. From the CFD simulations of the human gastrointestinal tract it is known that the Shear-Peclet Number from peristalsis is about 10-25. The in vivo environment has an order of magnitude lower shear-peclet range than the USP 2 paddle apparatus where Shear-Peclet number is at least 150.^{63-65,88,89} The Shear-Peclet number is dependent on particle size, so the intent of this design was to achieve the minimum possible shear imparted via the impeller to give flexibility in accommodating a wider range of particle sizes.

$${S}^{*} = \frac{S \times {R}_{p aτticle}^{2}}{{D}_{m}}$$

Where: S*=Shear-Peclet number, S=shear rate, Rparticle=radius of the dissolving particle, Dm=diffusion coefficient of the dissolving solid.

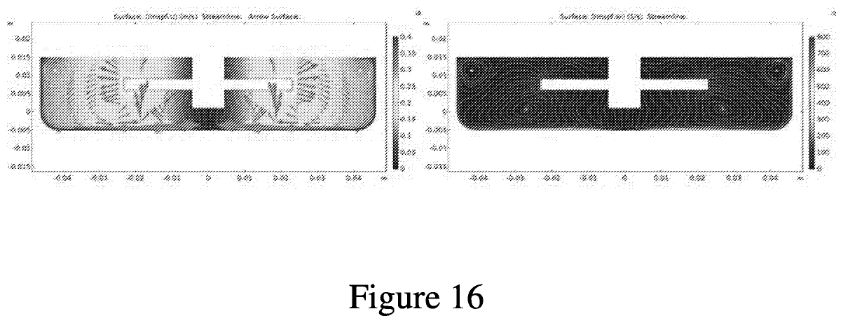

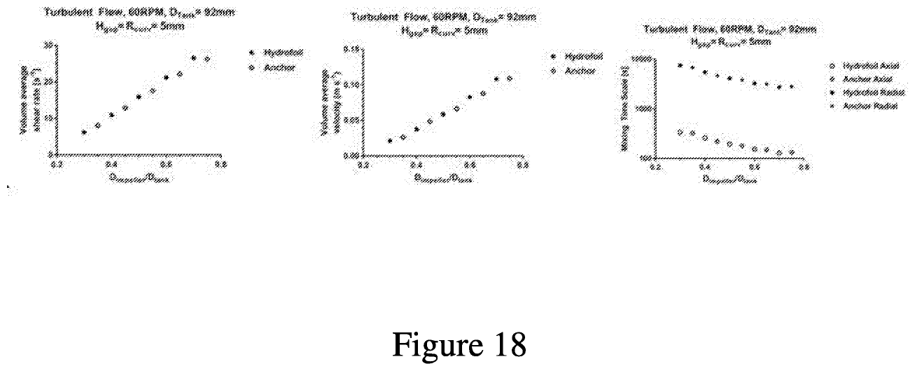

The ratio of the impeller diameter to the vessel diameter was also investigated to see their effect on bulk shear rate, fluid velocity, and the axial/radial mixing time scale (FIG. 18). Models of the impeller were constructed in Autodesk Fusion360 and imported into COMSOL where the appropriate model for the tank was constructed. FIG. 16 demonstrates that the hydrofoil in the asymmetric tank produces an axial flow. See Appendix A for cross section analysis of the shear and velocity profiles for all CFD plots.

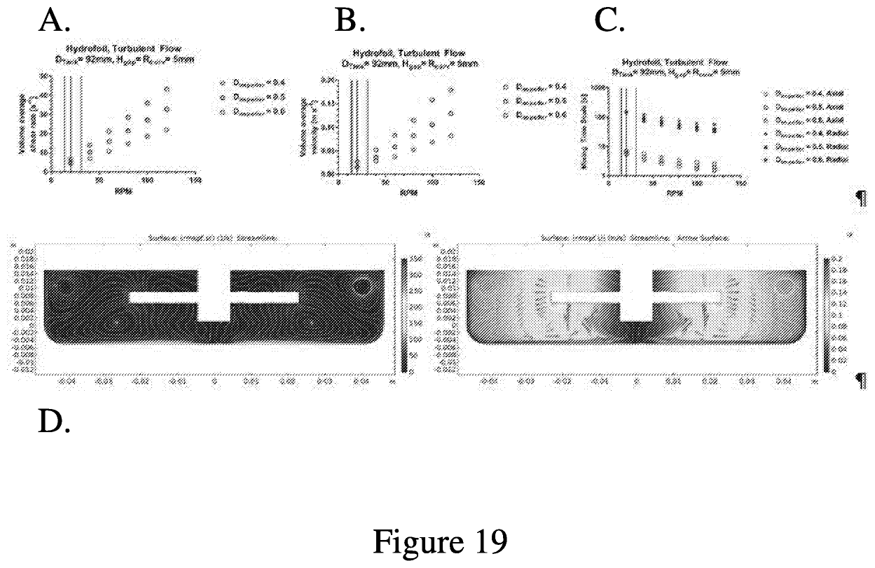

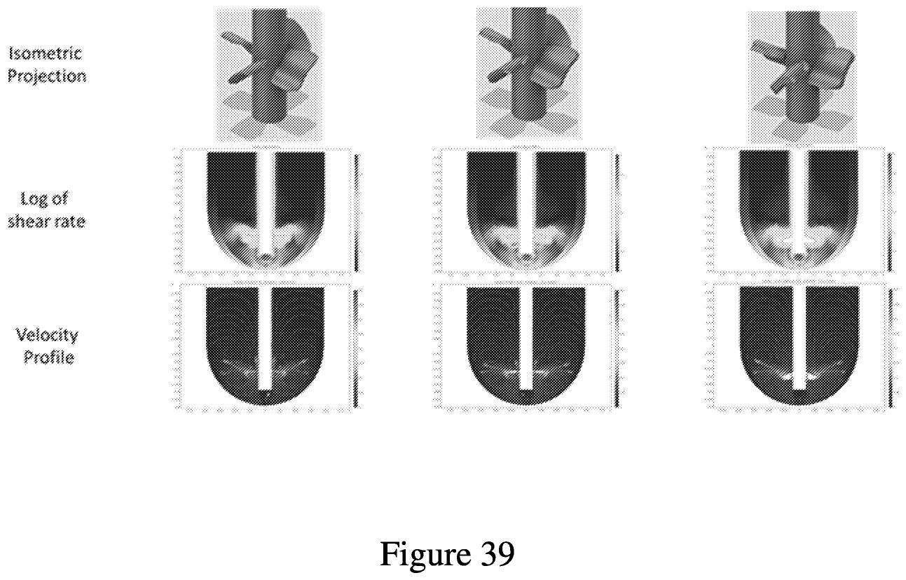

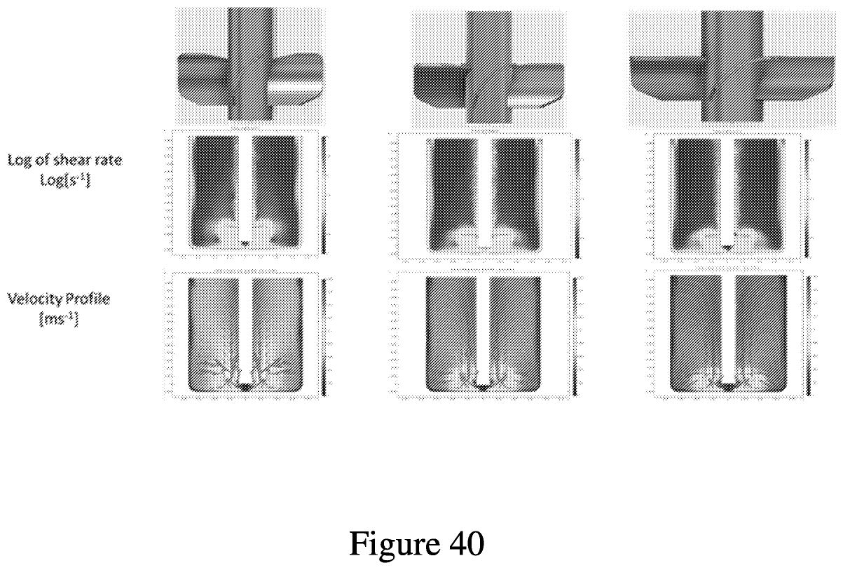

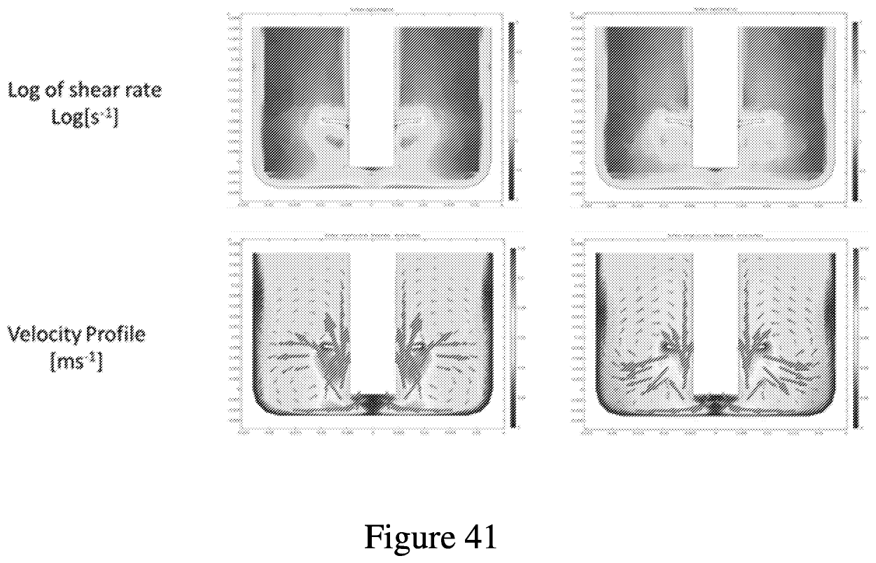

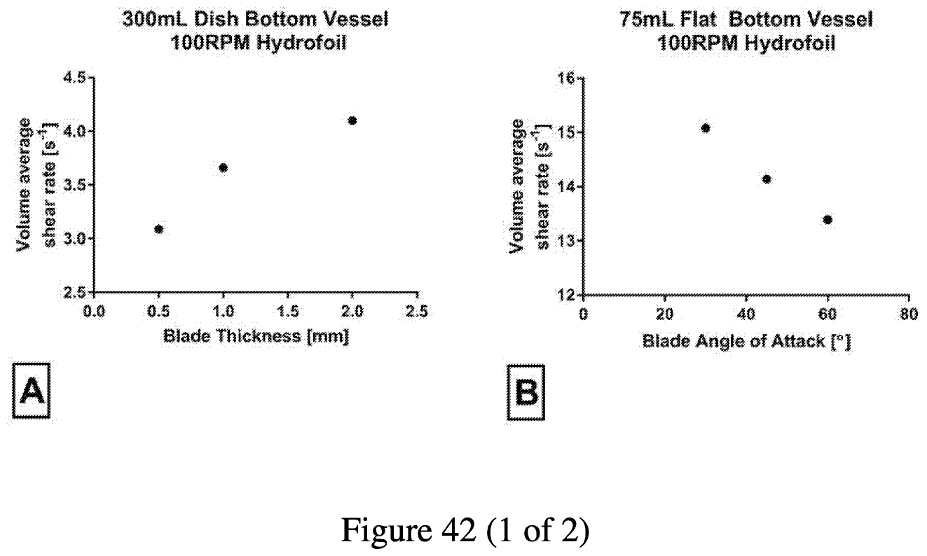

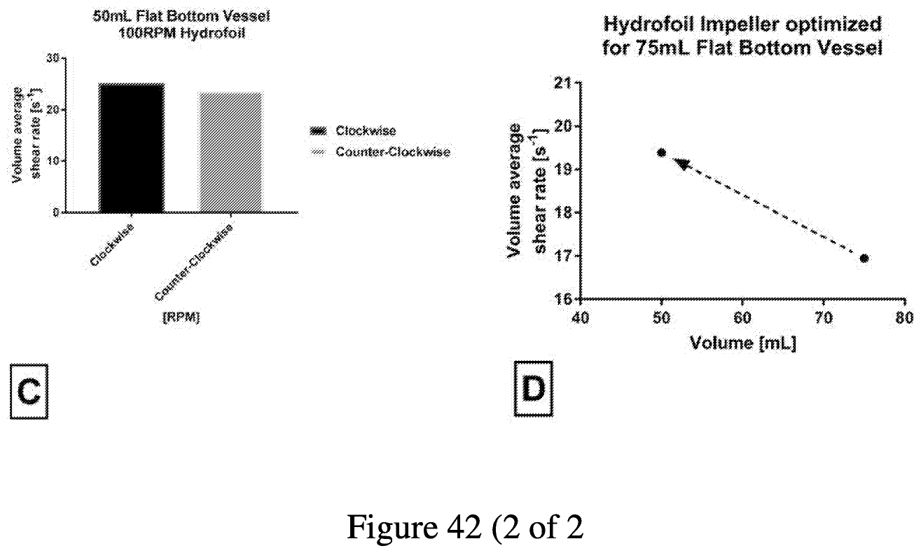

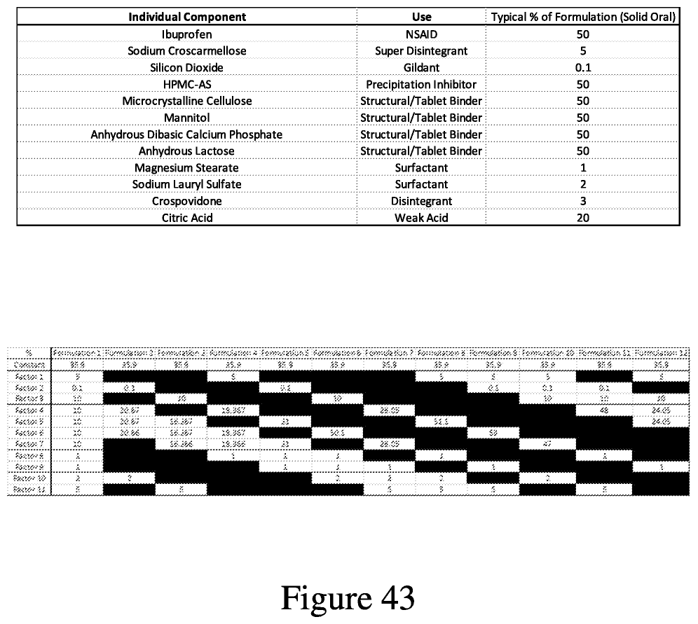

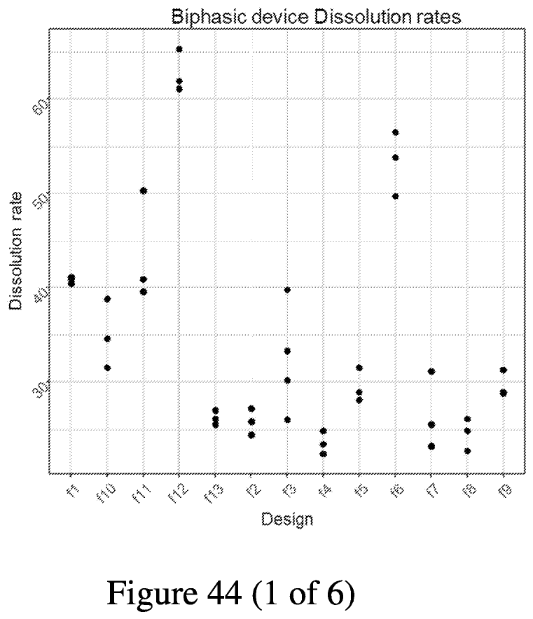

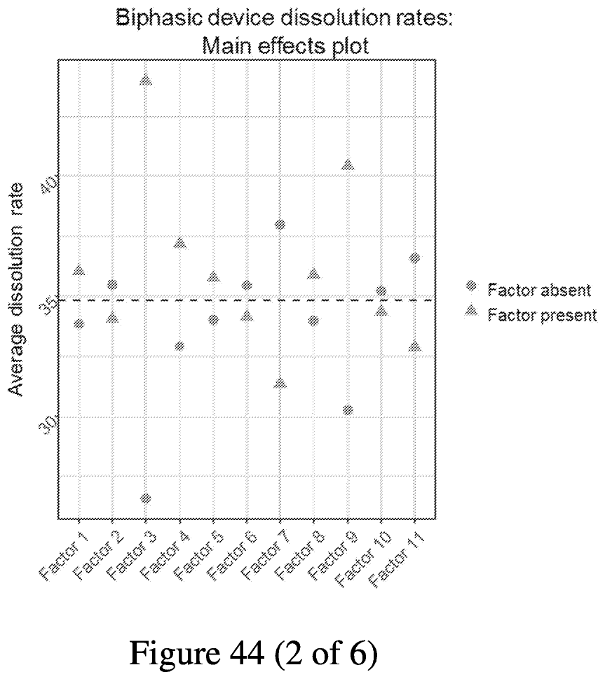

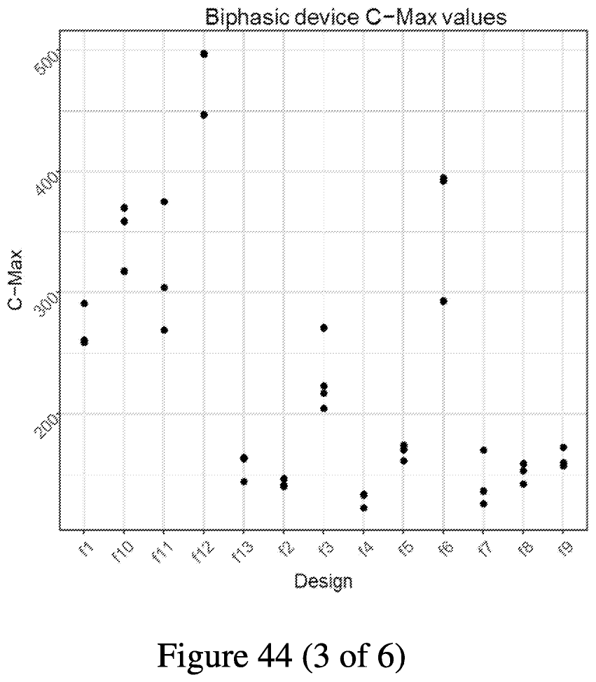

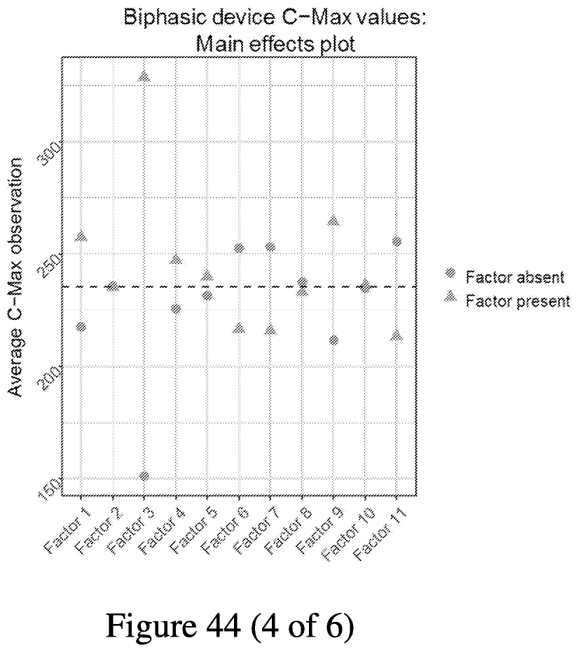

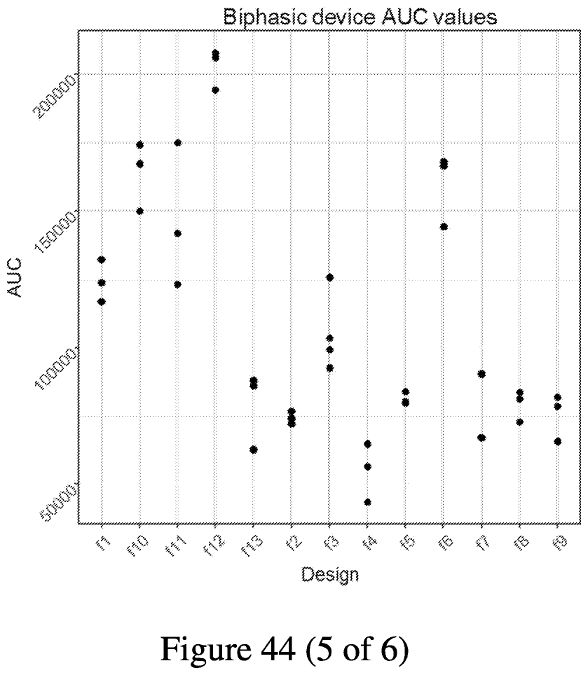

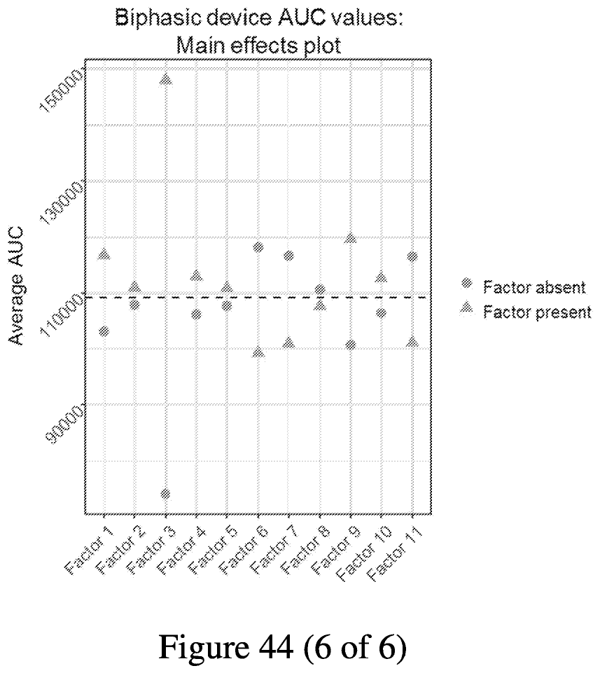

Based on the results shown in FIG. 19A-C, a Dᵢₘₚₑₗₗₑᵣ/Dₜₐₙₖ of 0.5 produced the lowest volume average shear rate, while maintaining significant velocity and low mixing time scales at a stirring speed of 60 rpm. This is similar to the Dᵢₘₚₑₗₗₑᵣ=0.3*Dₜₐₙₖ condition described as the optimal ratio.^{85, 87} The anchor impeller only provides radial mixing, and therefore it was not selected as a candidate for the final impeller in the UTLAM diffusion cell. Ultimately, the 0.5·Dₜₐₙₖ hydrofoil from the COMSOL simulations was replicated in CAD and fabricated on the Stratasys J750. (See FIG. 19D for cross section shear and velocity fields for one design embodiment.) Other computational studies were performed to see if there were any other parameters that could effectively change the hydrodynamic parameters of interest including: Blade Thickness, Pitch Angle, Stirring Direction, Effect of Variable Volumes on Volume Average Shear Rate/Velocity (see FIGS. 39-42).

Absorption Compartment

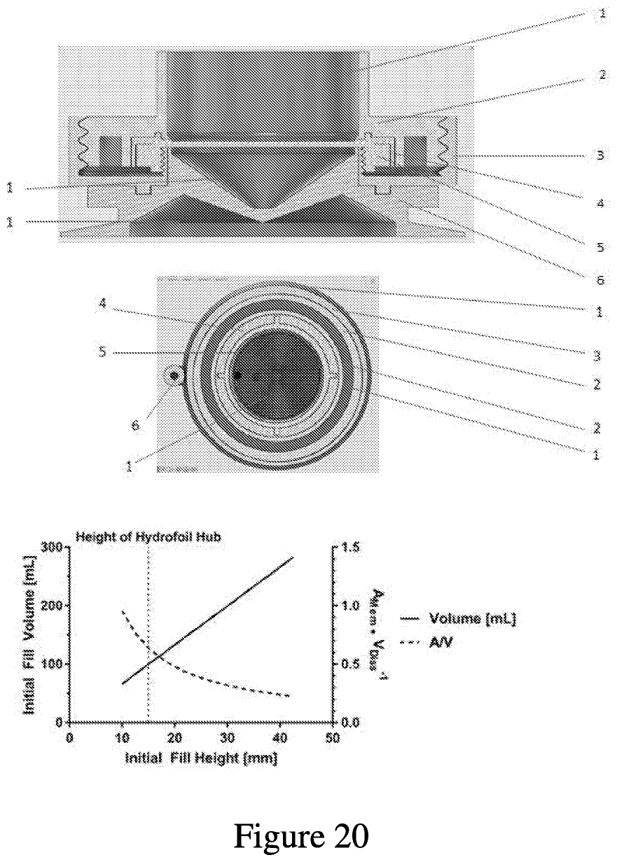

The absorption compartment resulted from many design decisions concerning the impeller and the dissolution vessel. For the UTLAM diffusion cell absorption chamber, a simple planar membrane at the bottom of the dissolution bowl was used. This geometry and orientation were simple to integrate into the design decisions discussed above. However, the disclosure contemplates configurations that take advantage of the side walls, or non-2D planar configurations. A mesh support was built at the interface between the dissolution vessel and the absorption chamber to support the membrane from below. Because the mesh support is only located underneath the membrane, the full surface area is accessible for transport from the dissolving drug particles. To select the best design for the dissolution bowl and the membrane surface area, the dissolution volume and surface area/dissolution volume were plotted against the fill height of the cylinder created by the aqueous fluid. FIG. 20 shows the absorption coefficient (A/V) and dissolution volume as a function of the fill height in the dissolution bowl. As currently designed, the impeller hub (cylinder that holds the hydrofoil blades) is 15 mm tall, and the gap distance is 7 mm. This means that the initial UTLAM is just on the right side of the “intersection” point of the volume and A/V curves in FIG. 20, leaving room for other UTLAM diffusion cell embodiments having advantageous or optimized absorption capabilities. A rendering of the X and Y planes and the membrane plane are also shown in FIG. 20.

### Example 6

Prototype Fabrication and Evaluation

Fabrication of Ultra Thin Large Area PDMS Membranes

Prior to the spin casting technique, PDMS membranes produced by drop casting were only as thin as about 150 micron due to the propensity for the PDMS to self-adhere and tear under the applied stresses during the separation process. Another method of fabrication was required to achieve thicknesses below 150 micron while maintaining uniform thickness, and so the spin coating process was chosen. Even though PDMS is not a delicate material, at the cross sectional length scale targeted for fabrication (1-60 μm, E_{1 μm}, =8 MPa, E_{60 μm}=2 MPa)⁴⁷, it would be time consuming to remove such a high aspect ratio structure from its casting substrate as the shear modulus appeared to be much lower than the elastic modulus, rendering the membrane susceptible to mechanical failure under shear stresses. Therefore, an accelerated removal process was implemented using a water-soluble sacrificial layer composed of PVA, which also removed any significant mechanical force needed to separate the membrane from the casting substrate. A nanoscopic layer of PVA was deposited from a 3% w/w PVA in deionized water solution at 5000 rpm onto a silicon wafer that had been rinsed with deionized water and dried thoroughly prior to casting. The solution fully coated the stationary silicon wafer but was not allowed to sit stationary for more than a few seconds to avoid adherence of the polymer to the surface, which could lead to heterogeneity. The ellipsometer was used to characterize the thickness of the PVA layer on the silicon. Twenty wafers were measured at five consistent points (approximately 5, 33, 50, 67, and 95% of the distance along the major diameter).

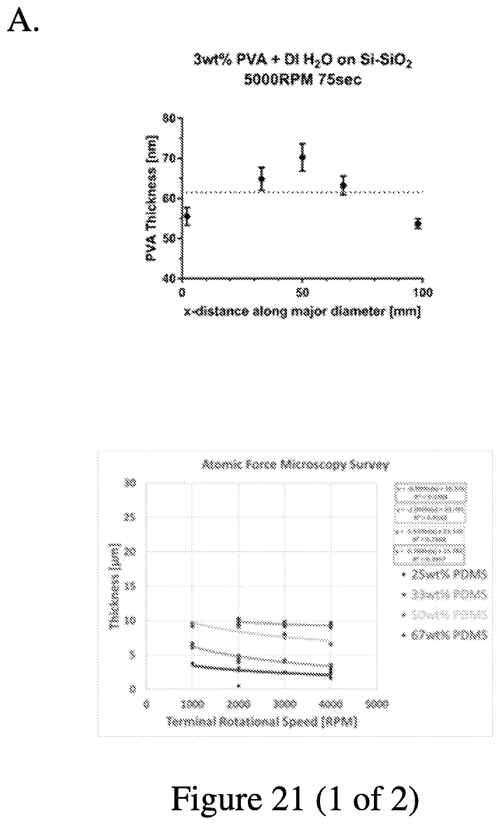

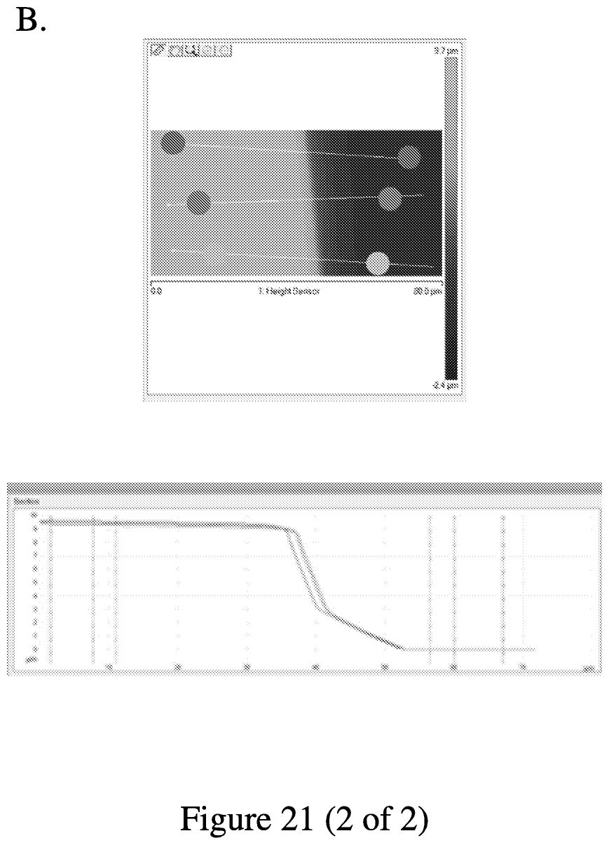

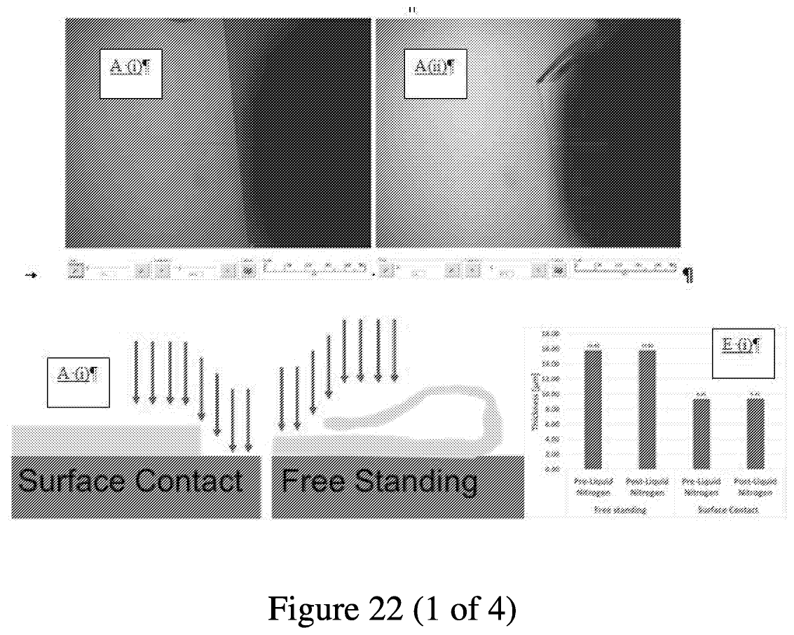

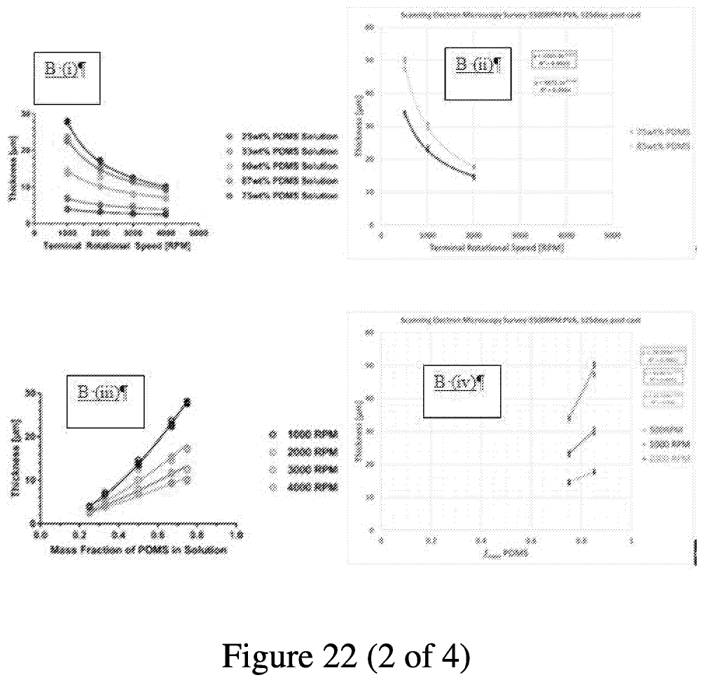

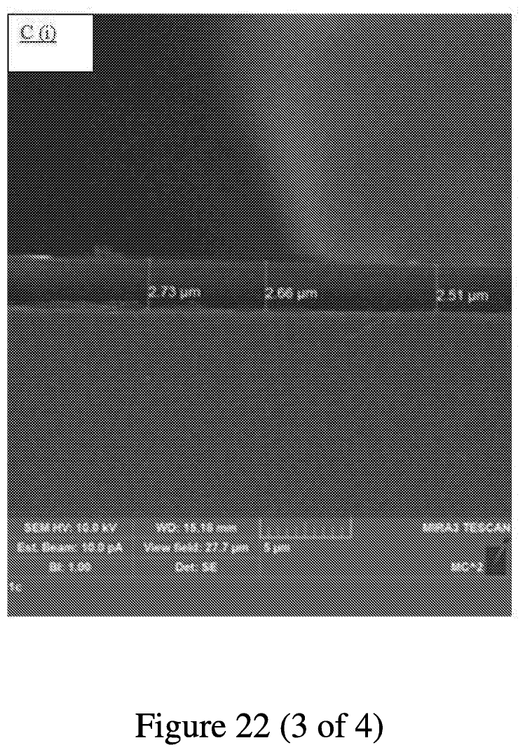

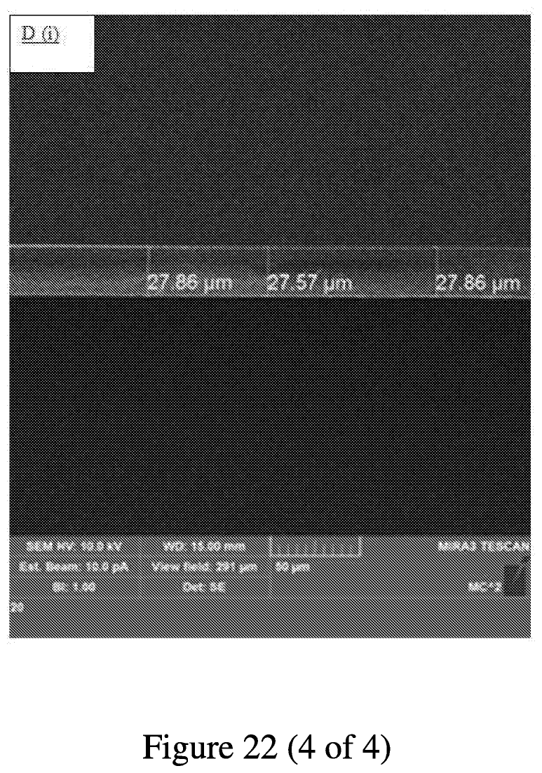

Ellipsometry proved to be a fast and convenient tool to measure the PVA films, but the tool cannot reliably measure films thicker than about 3 microns. Therefore, another technique was required to measure the thickness of PDMS. One technique that was investigated was atomic force microscopy (AFM) in the soft tapping configuration (FIG. 21). Sample wafers were scored on the etched side of the wafer with a diamond scriber and fractured along the trace using a glass slide as a fulcrum. The silicon undergoes brittle failure, but at room temperature PDMS is elastic, so PDMS remained intact post-fracture. Once the silicon was fractured, the PDMS was shorn rapidly in the plane perpendicular to the newly formed edge. The results of the AFM study were inconsistent with the behavioral responses to changes in terminal rotational velocity and solution concentration of polymer observed in the literature. It was hypothesized that the room temperature preparation of samples was altering the edges of the membrane being scanned by the AFM, specifically that during the shearing process PDMS would elongate and fail to recover its original shape due the strong interaction forces between the polymer and the substrate. AFM measurements of edges were compared to free-standing flaps of PDMS and a significant difference in thickness was observed (FIG. 22).

Once the preparation method of freeze-fracturing the wafer composite was confirmed not to influence the sample, samples were re-processed by forming new edges on the original AFM samples, which were then examined with a scanning electron microscope (SEM). The SEM results demonstrated that the liquid nitrogen freeze-fracture technique prevented any unintentional plastic deformation of the sample edge and allowed for a clean brittle fracture to propagate from the silicon wafer through the PDMS layer. The results of the SEM study showed similar trends in thickness change with changing solution concentrations and rotational speeds as reported in the literature for spin coating polymer solutions. This study led to two conclusions. The first was that samples would have to be prepared under plastic conditions to prevent modification of the PDMS membrane during preparation. The second conclusion was that even though the wafer composite was vacuum-annealed for 24 hours at 65° C., residual compressive stresses remain in the PDMS from casting, causing significant diameter reduction in the membrane.

### Example 7

Plackett-Burman DOE for Blended Uncompacted Solid Oral Dosage Forms Containing Standard Excipients at Commonly Used Levels

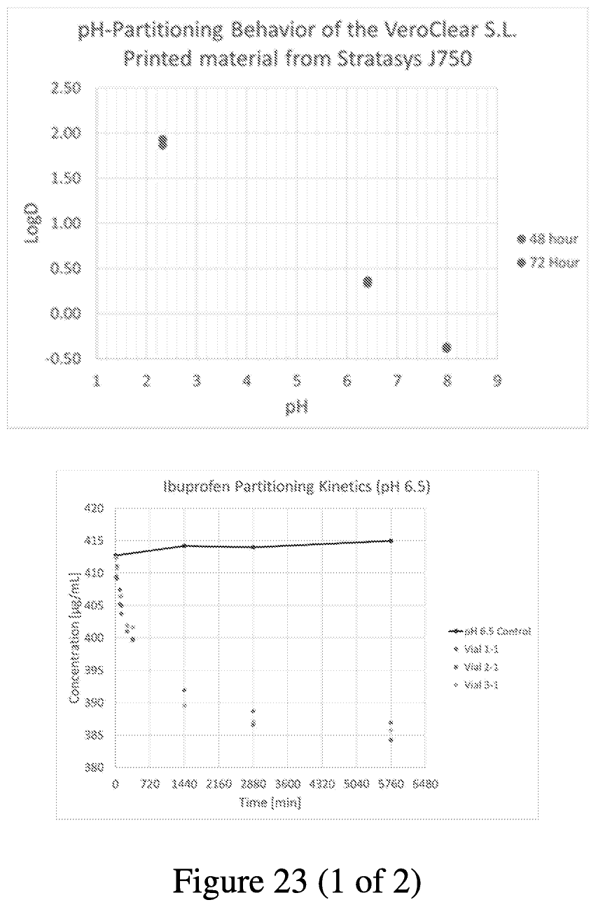

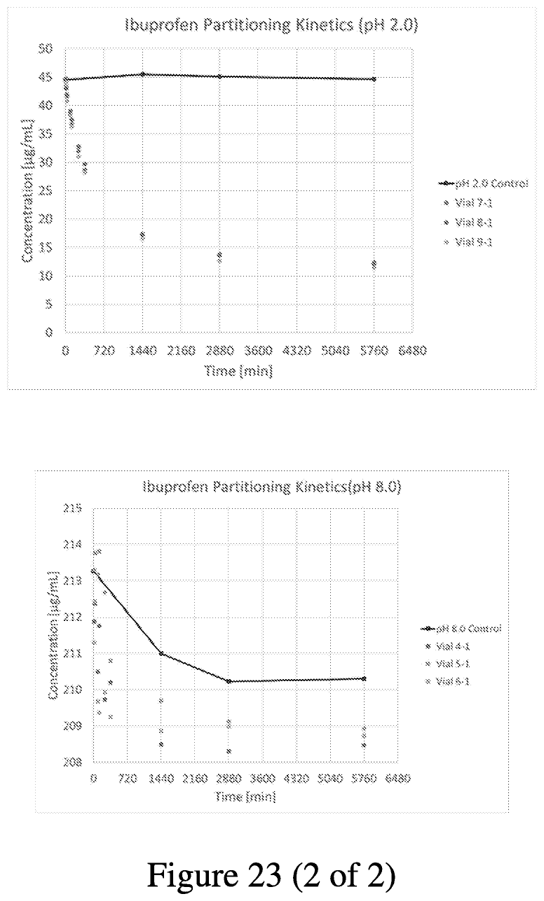

The UTLAM diffusion cell was evaluated for solvent compatibility and partitioning affinity so that the chemical performance of the SL printed VeroClear material could be established. It was established that the VeroClear material had significant partitioning ability and that the device could withstand exposure to aqueous buffers and cleaning solvents (e.g., methanol) (FIG. 23). The UTLAM diffusion cell was leak-checked using an ibuprofen solution and a pH meter. No leak was detected and the UTLAM diffusion cell was then ready to begin testing formulations with a 57 μm membrane in place.

Experiments were designed to assess whether absorption kinetics are important for API with significant lipophilicity and significant differences in in vitro parameters that could be better measured by dissolution systems with absorption components. It was important to obtain these experimental results because the UTLAM method of incorporating absorption is easier, more cost effective (no need for filtering receiver phase samples, could be compatible with UV-dip probes which provide more data, real-time, and reduce HPLC throughput demands), more environmentally friendly, and more pleasant to work with as compared to the biphasic aqueous-organic solvent test. The experimental design incorporated 11 common ubiquitous excipients in solid oral dosage forms, with the typical levels of each excipient identified. The marker was ibuprofen and ibuprofen's dissolution rate, area under the curve (AUC), and absorption rate (where applicable) were measured for each formulation in a standard USP 2 900 mL dissolution test, a 200 mL/200 mL aqueous/1-octanol Biphasic dissolution test in USP 2 vessel, and the 130 mL donor/100 mL receiver UTLAM dissolution test in 50 mM phosphate buffer, pH 6.5. This design allowed for a Plackett-Burman 11 factor in 12 runs analysis with the addition of a 13^{th} run to serve as a negative control (drug only, no excipients). Plackett-Burman partial factorial arrays are efficient experimental designs for screening in which main interactions between factors can be studied rapidly; however, the second order and higher interactions are confounded.⁹⁰⁻⁹² In such cases, a different experimental design must be used once the main factors of interest are identified and further investigation of the higher order interactions becomes necessary.

The excipients chosen from this study represent most major excipient functions in modern solid oral dosage forms at typical compositional levels (FIG. 43). Microcrystalline cellulose, mannitol, anhydrous dibasic calcium phosphate, and anhydrous lactose are common structural excipient which can compose much of the formulation. Citric Acid was to acidify the local solution. Sodium croscarmellose and crospovidone are polymer based disintegrants which act via water uptake and swelling to burst the compacted solid. The formulations in this study were uncompressed powders, so the expected advantages of formulations with disintegrants was muted because there is no tablet to break apart. However, there could be solution phenomena such as aggregation of polymer at the absorption interface (octanol or PDMS membrane) which could alter the partitioning rate. Sodium dodecyl sulfate is a surfactant which could also have the potential to interact with the absorption interface. Magnesium stearate and silicon dioxide are common lubricants/glidants, respectively. HPMC-AS is a precipitation inhibitor polymer. For each iteration of the formulation, the non-structural excipients were held at a constant level which would be consistent for the typical amount of that excipient in a formulated product. The difference would be evenly distributed among any structural excipients in the formulation.

To conduct the experiment, 5.4 L of phosphate buffer was initially degassed and then heated to 37° C. in the USP 2 six-station apparatus. During the heating process, the motor was engaged, allowing for the fluid to be stirred at 50 rpm for no less than 30 minutes prior to dosing the formulations. Formulations were weighed on an analytical balance prior to dosing and were administered through ports in lids on the USP 2 bowl. The experiments ran for one hour and at the end of the experiment, 100 mL of acetonitrile was added to solubilize any undissolved ibuprofen. The acetonitrile/phosphate was then allowed to run for an additional hour, at which time the mass balance sample was taken. Two mL samples were drawn and media was not replaced for the USP 2 monophasic experiments. One mL of sample was discarded through a 0.45 μm PVDF syringe filter and then one mL of sample was captured and diluted 1:1 in acetonitrile for HPLC analysis.

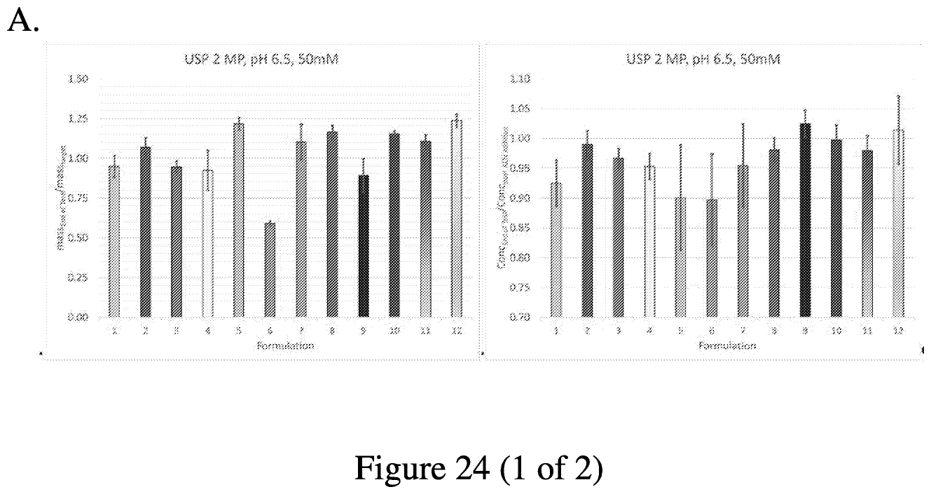

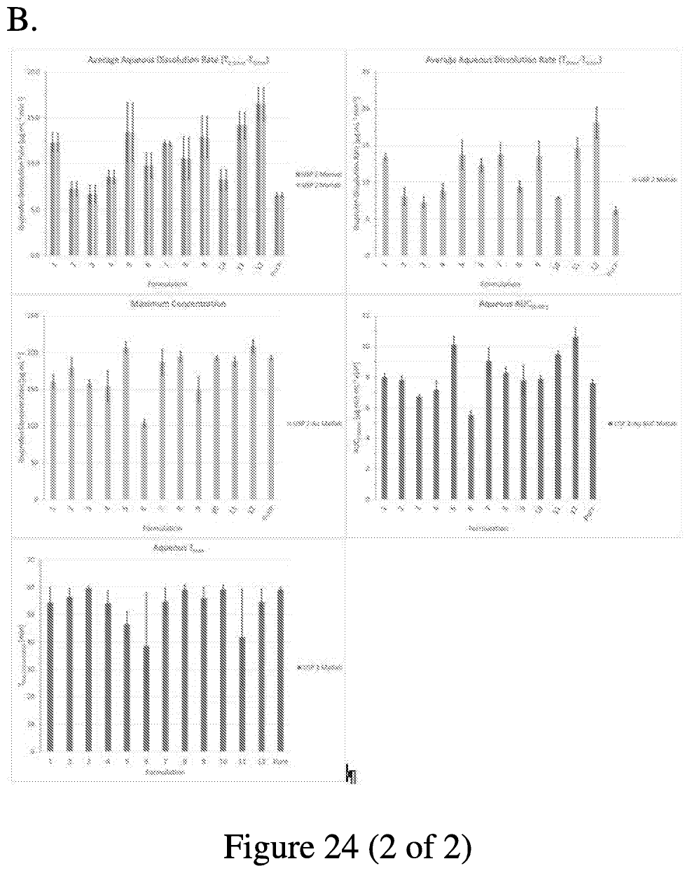

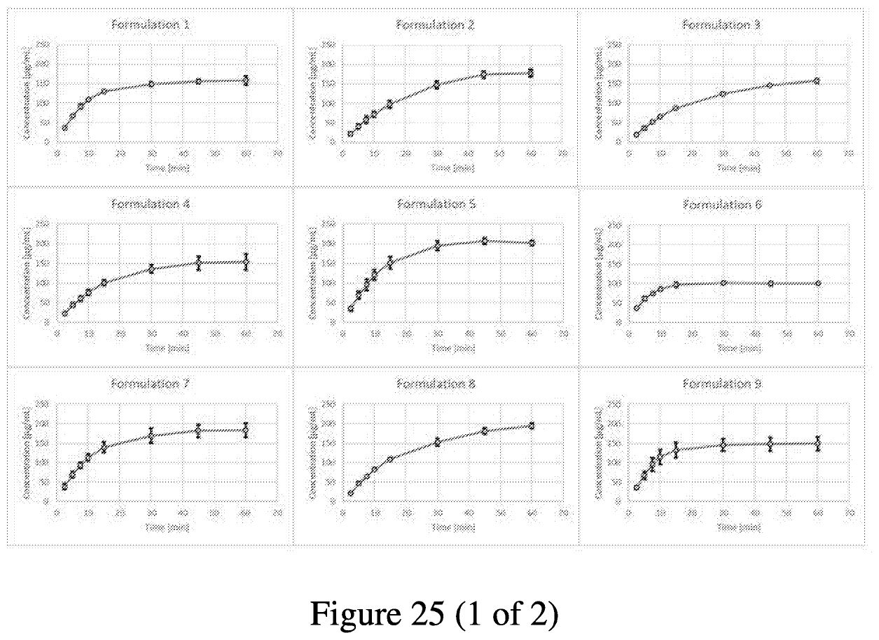

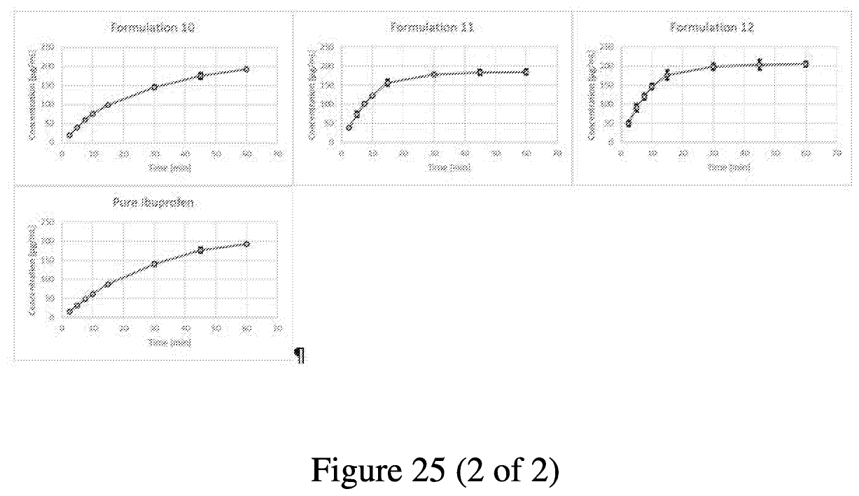

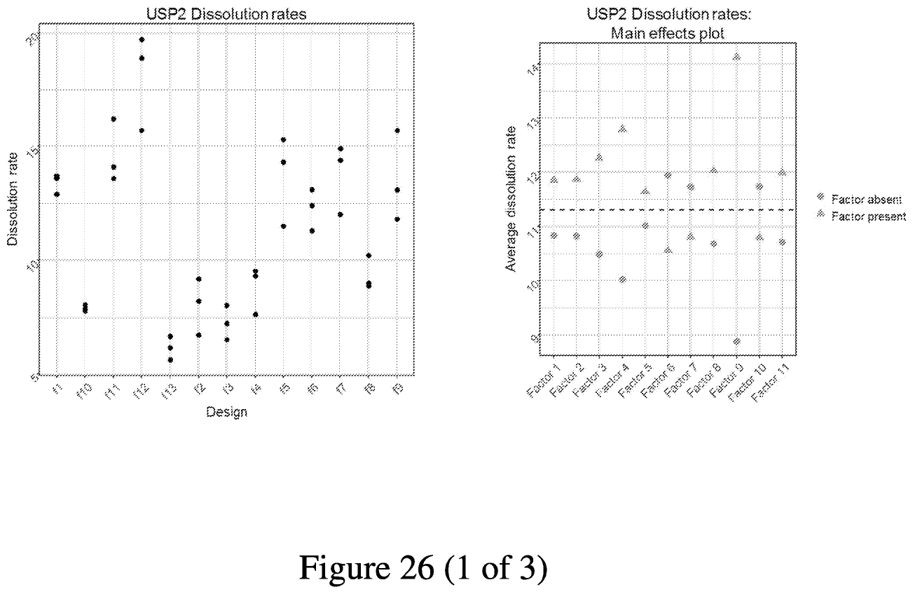

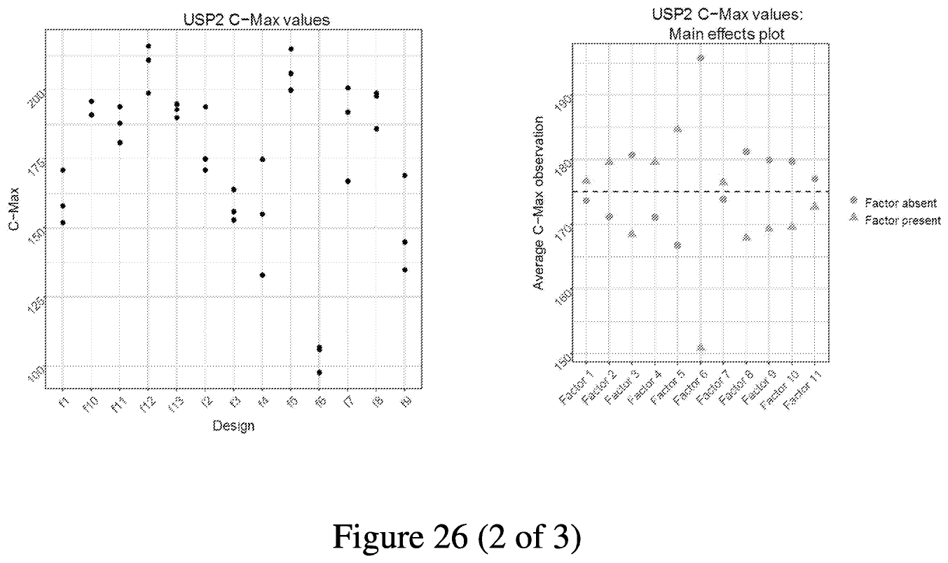

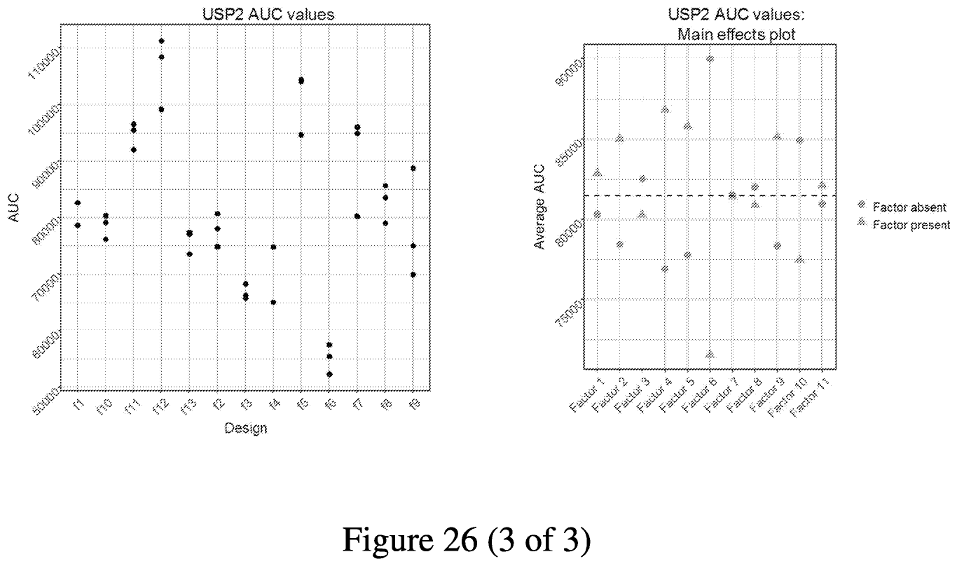

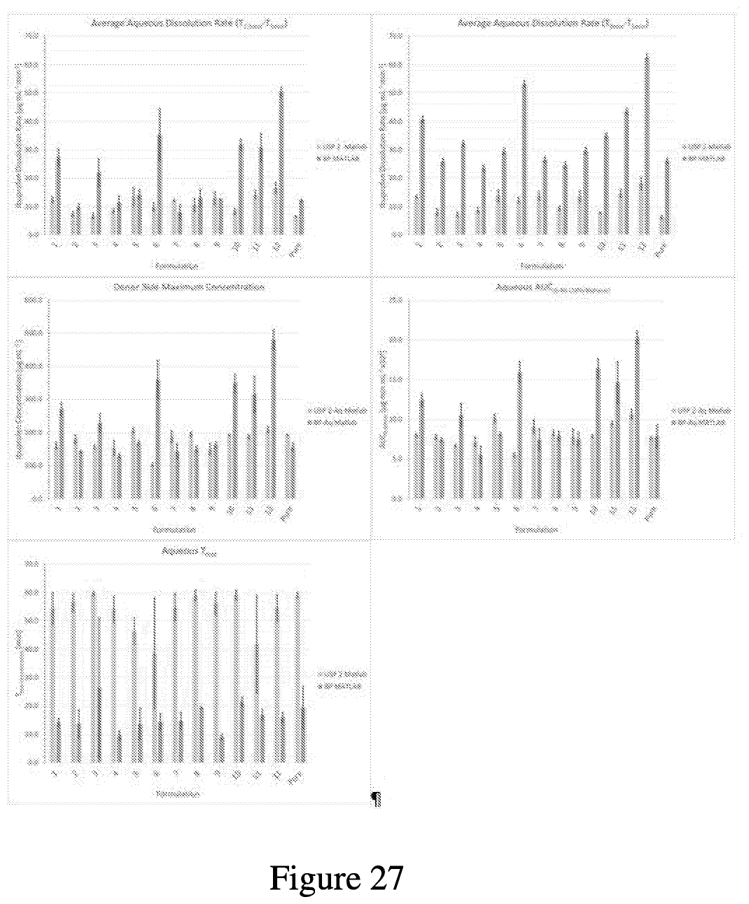

The HPLC data was converted from peak area to concentration with a standard curve and then the time course data was input to a MATLAB program that fit the data with a spline function. This allowed for better estimations of Cₘₐₓ and tₘₐₓ when the Cₘₐₓ and tₘₐₓ did not fall within the first 10 minutes, as well as allowing for rapid calculation of the AUC_{0-60 min} via a numerical trapezoidal method. This program was applied to the data of all three apparatuses and a comparison of the manual and MATLAB method can be found in FIG. 24. The concentration profiles for each formulation are provided in FIG. 25.

### Example 8

USP 2 200 mL/200 mL Biphasic 1-Octanol/Water Dissolution Experiment

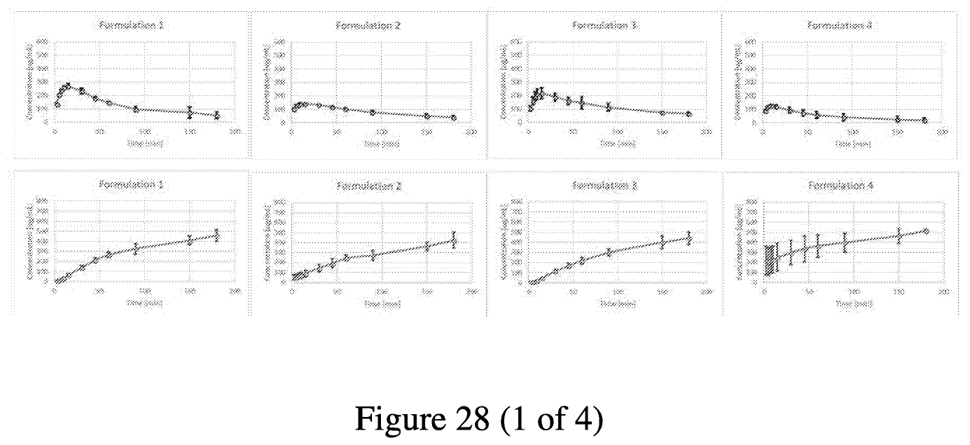

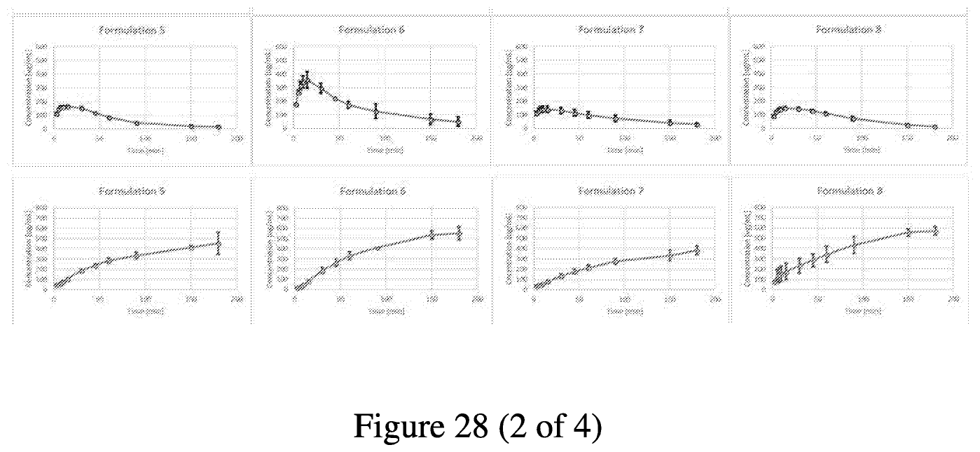

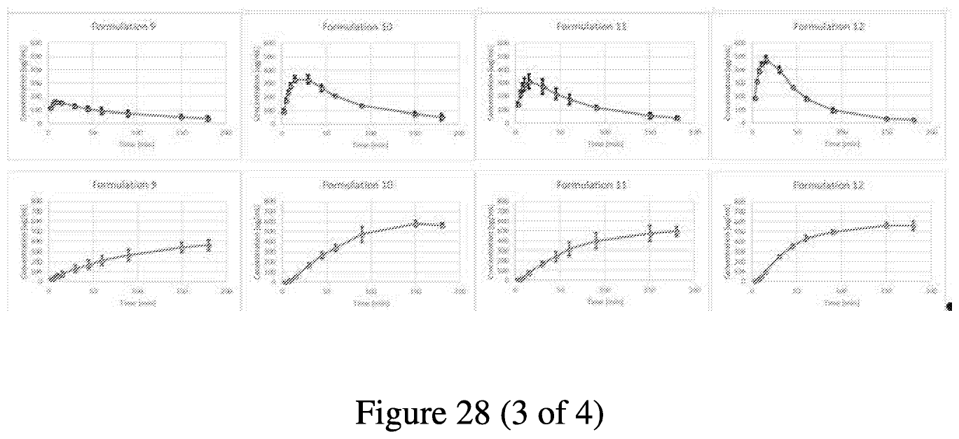

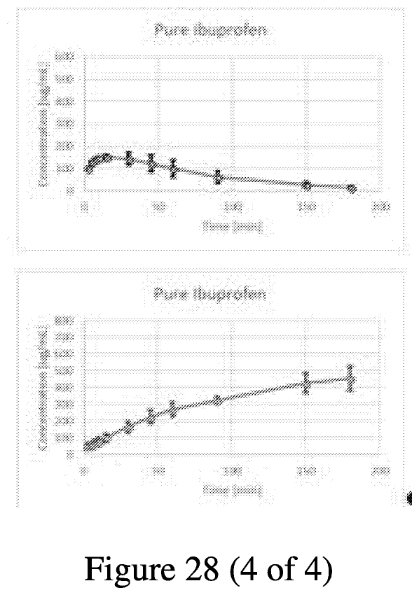

In preparation for the dissolution experiment, 200 mL of phosphate buffer was degassed and heated in a single, water jacketed USP 2 bowl. A custom lid cover was created to house the sampling ports for the aqueous and organic phases, as well as house a large diameter tube to dose the solids directly to the aqueous phase after the organic phase had been poured on top. The advantage of this approach was that the two phases equilibrated prior to the dosing of the solid, as opposed to having to rapidly fill the organic phase immediately post-dose. The tip of the dosing tube was far enough under the aqueous surface that no organic could enter the tube (even under the pressure applied by the 1-octanol) and the tip was close enough to the top edge of the USP 2 paddle that a large shear could pull powder down into the vessel without any experimenter assistance. The dosing tube was constructed from two 10 mL pipette tips, which were wide enough to prevent any “rat holing” or other powder-flow concerns. The aqueous media was gently poured down the sides of the dosing tube to replenish media removed from sampling to catch any solid that may have stuck to the tube during initial dosing, and the 1-octanol was carefully injected through the organic sampling cannula to avoid disturbing the organic-water interface with bubbles. Two milliliter samples were drawn from each phase and filtered with a 0.45 μm PVDF syringe filter. The respective media were replaced in each phase post-sampling. The dissolution and partitioning profiles are shown for each formulation in FIGS. 27-28.

The biphasic device had significantly varied performance when compared to the counterpart USP 2 experiments (FIG. 44). Dissolution rate, maximum concentration, and AUC were significantly affected by each excipient in the formulation. This system is not the UTLAM system, but it very clearly demonstrates that even simple formulations and simple drug molecules can exhibit drastically different performances in the in vitro dissolution device when comparing a traditional dissolution method to an in vivo relevant (ivR) dissolution method. Another significant result of this type of comparison was apparent in that, not just the magnitude of rates and areas that were different, but the performance of the excipients themselves changed in between test methods. For example; factor 6 (anhydrous dibasic calcium phosphate) in the USP 2 device was practically the only excipient that had an effect on the performance of ibuprofen, whereas in the biphasic device there was almost no response from anhydrous dibasic calcium phosphate.

### Example 9

Ultra Thin, Large Area Membrane (UTLAM) Conventional Dissolution Experiments

Experiments using the VeroClear SL printed prototype UTLAM diffusion cell used the standard 162 mg dose mass of ibuprofen. The parts were soaked in deionized water (milliQ), then vigorously scraped to remove residual support material. To measure the partition coefficient of the VeroClear resin, 10 mm diameter spheres were printed, cleaned, and then exposed to 19.5 mL of 400 μg mL⁻¹ solution of ibuprofen in a 50 mM phosphate buffer at pH 6.5 for 48 hours. The dry mass and diameter of the spheres were recorded prior to the beginning of the partitioning study. The results of the equilibrium partitioning experiments are presented in FIG. 23. Concurrent to the partitioning experiments, a USP II vessel was fabricated in the J750 to USP specifications. When the standard USP II vessel results were compared to the results from the J750 fabricated USP II vessel, VeroClear resin was seen to have a significant partitioning ability, as observed by the increase in dC dt⁻¹ and the decrease in Cₘₐₓ consistent with observations of ibuprofen performance between the compendial USP II apparatus and the biphasic experiments reported herein. There was ibuprofen present in the water bath used to test the printed USP II device that was able to partition into the virgin resin and accumulated to 3 μg/mL in the 900 mL of phosphate prior to the dosing of ibuprofen crystals. Another difference between the two devices is the surface roughness, which could influence the hydrodynamic parameters of the particle dissolution. MATLAB simulations were run to see if the compendial dissolution test could be predicted and if the partitioning kinetics of the VeroClear (once determined) could be properly accounted for in the MATLAB model.

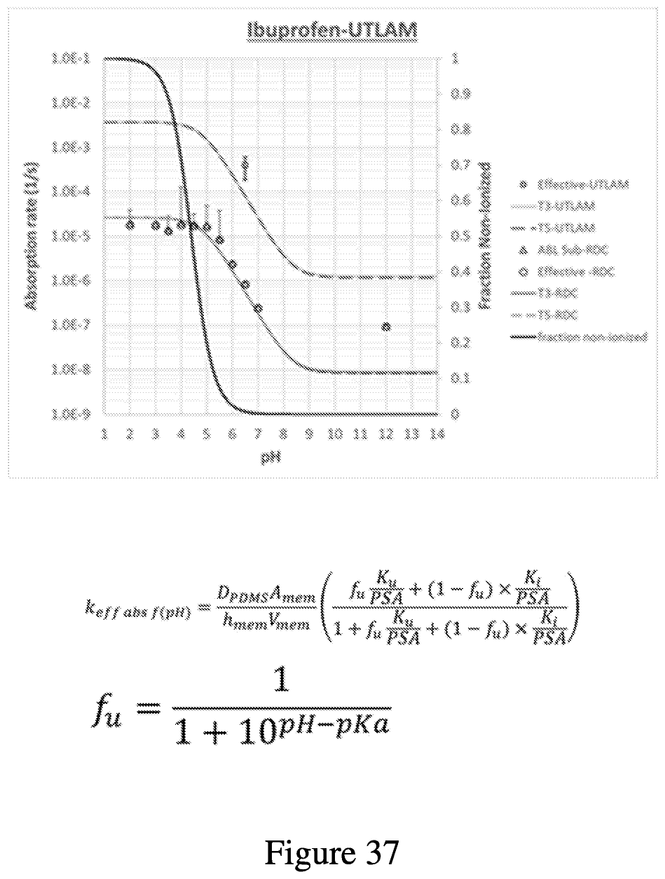

According to the SEM freeze-fracture-determined thickness based on the terminal rotational speed and mass fraction of PDMS, the membranes tested in the following experiments were 57 microns thick. 130 mL of 50 mM phosphate buffer pH 6.5 was degas sed and used as the donor phase, while 100 mL of 50 mM phosphate buffer pH 8.0 was degas sed and used as the receiver phase. The rationale behind this is that PDMS has poor ability to transport ions (a significant pH-partition relationship) and the more ionized the drug is, the less driving force the drug will provide for reverse transport out of the receiver phase. However, with this set of pHs, the pH-distributed partition coefficient (referred to as Log D) of ibuprofen in both compartments will not have a large difference, leading to a nearly equivalent permeability on both sides. Ultimately, this leads to a significant transport rate of ibuprofen returning to the donor compartment. Even with this bi-directional flux, the net flux yields an absorption rate for ibuprofen that is within the same order of magnitude of ibuprofen absorption in human beings. It is understood that the measured absorption rate is increasing due the additional partitioning kinetics introduced by the resin that forms the UTLAM device. FIG. 37 demonstrates that the PDMS UTLAM-only rate is lower than this rate but still much higher than thicker membrane systems used in the past with smaller surface areas. Physiologically, it is understood that the bicarbonate buffer bulk pH and buffer capacity are lower in the duodenum and jejunum than that of the USP 2 dissolution test. The duodenum and jejunum are where most drug absorption is expected to occur for standard drug formulations. However, to be consistent with previous experimental methods, the UTLAM dissolution experiments where performed with 50 mM phosphate buffer pH 6.5 as a donor phase. Preferably, a bicarbonate buffer or an equivalent phosphate buffer with lower pH and buffer concentration would be used in truly in vivo relevant (ivR) applications of the UTLAM experiments.

The PDMS UTLAM is floated in deionized water and in about two hours the PVA is dissolved enough to tease the PDMS UTLAM to the surface of the water. The membrane is then moved across the water surface to the support mesh and the water is drained from the vessel allowing the PDMS UTLAM to settle onto the mesh support structure without handling the UTLAM or the support is brought beneath the UTLAM and positioned with tweezers or other handling device, then lifted from the water. This support structure screws into the central hub of the UTLAM diffusion cell and then the dissolution bowl and absorption chamber can be assembled. The receiver phase is filled first, with the aid of an accessory design to compress the membrane during filling to prevent mechanical failure. Then, the receiver phase is cleared of bubbles and the donor phase is poured into the dissolution bowl. The hydrofoil is rotated at 50 rpm to be consistent with the impeller rotational speeds in the monophasic and biphasic dissolution experiments. Equivalent stirring rates could be calculated for the USP 2 paddle using CFD measurements made in COMSOL. The UTLAM diffusion cell was then placed in a water bath with immersion heater (sous vide heater) to adjust the aqueous phase temperature to 37° C.

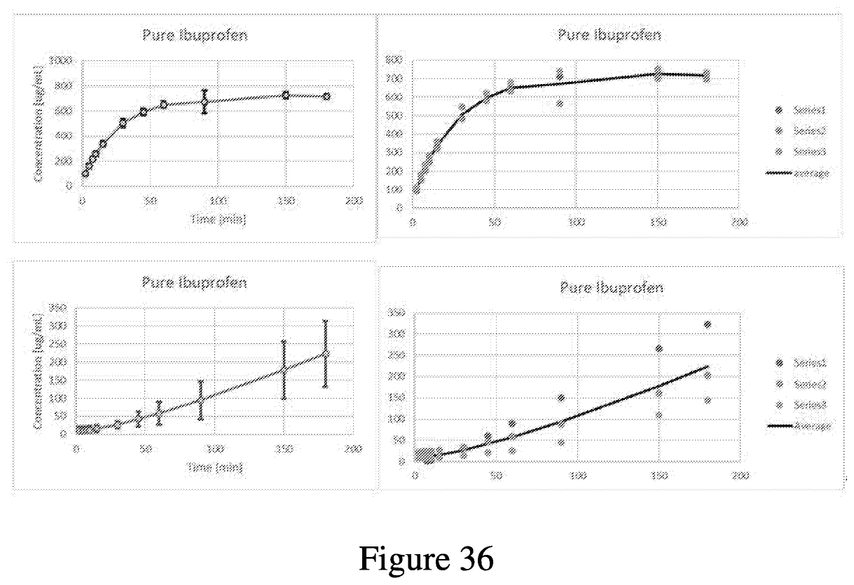

Re-use of membranes was also examined. Experiment 1 provided a control in assessing the behavior of virgin PDMS UTLAM and the expected j-shaped curve (indicating a small lag time) was observed. Experiments 2 and 3 were conducted using the same experimental conditions except for the membrane, which was the membrane that was used in experiment 1, but washed with deionized water and methanol in between experiments. The permeability measured from the pseudo-steady state region decreased with re-use, but the initial concentration decreased as well (FIG. 36). This indicated that there was retained drug in the membrane or apparatus itself, which then was extracted by the new dissolution media in each experiment. The experiments with the SL printed USP II vessel demonstrated that there was significant partitioning from the water bath through 5 mm of the VeroClear resin.

The permeability of ibuprofen in the UTLAM was consistent with predictions in FIG. 37 of required permeability to achieve human magnitude absorption rates in the UTLAM diffusion cell. The UTLAM device produced results within expectations based on earlier characterization and a model that is independent of the device used to collect the data.

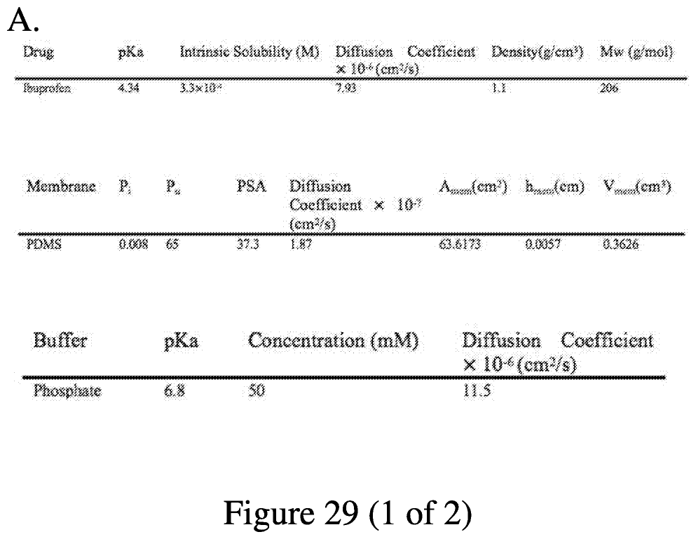

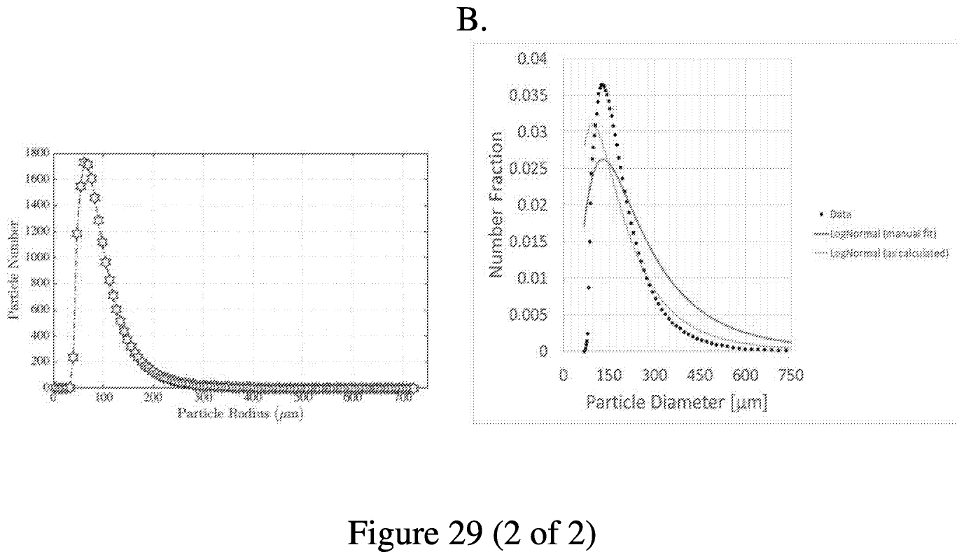

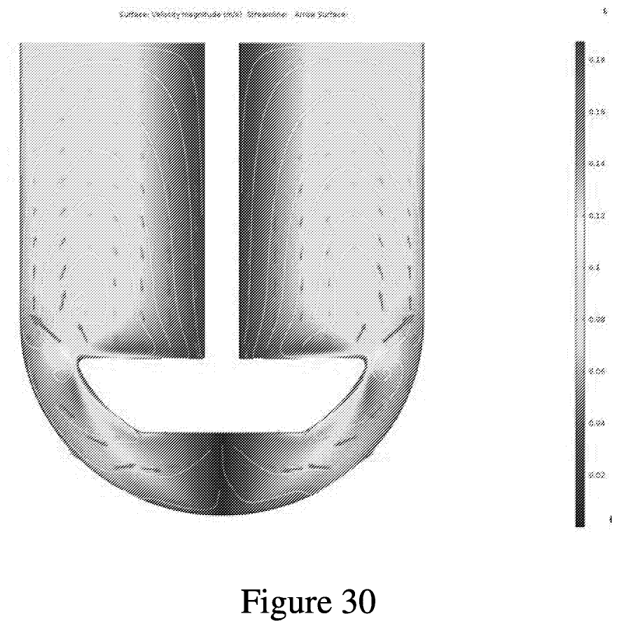

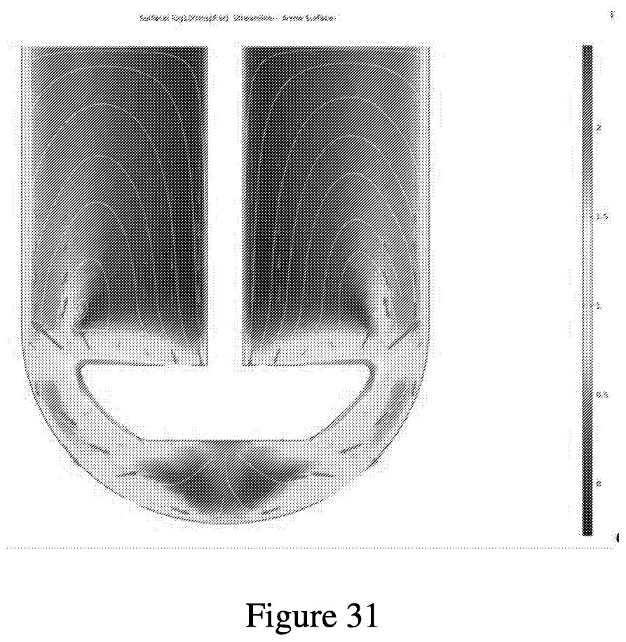

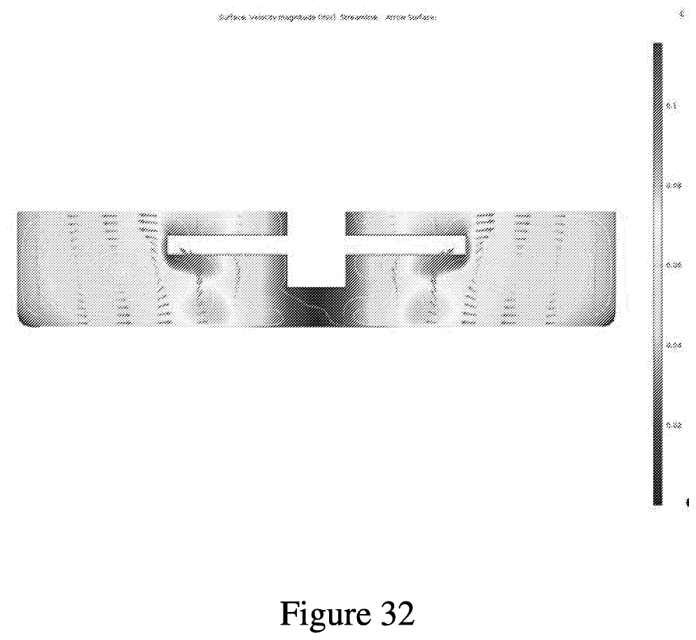

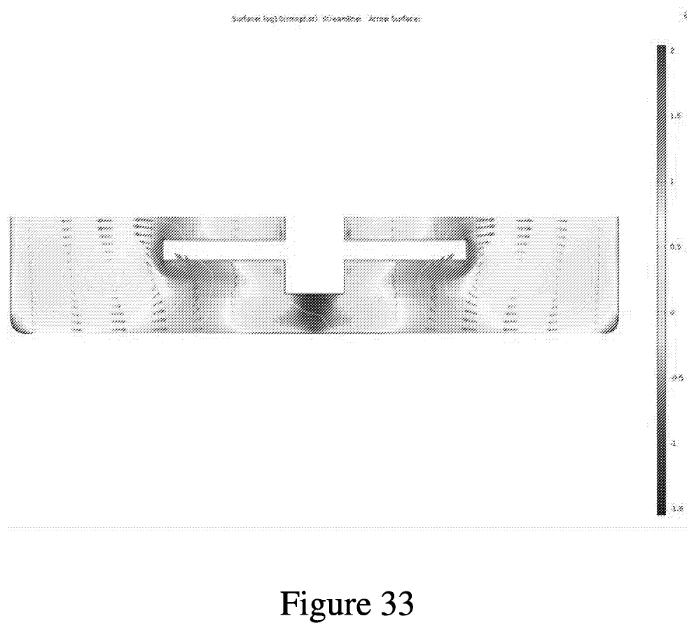

Simulations of ibuprofen dissolution in the UTLAM were run in COMSOL and in MATLAB to confirm that all the experimental data and the mechanistic models were consistent, in addition to being able to apply the hydrodynamic parameters measured via CFD. See FIG. 29 for the hydrodynamic parameters used to simulate the USP 2 and UTLAM dissolution experiments via COMSOL CFD.

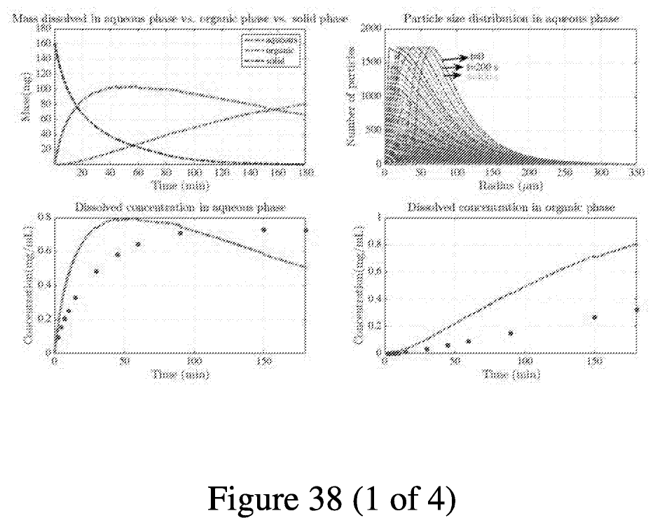

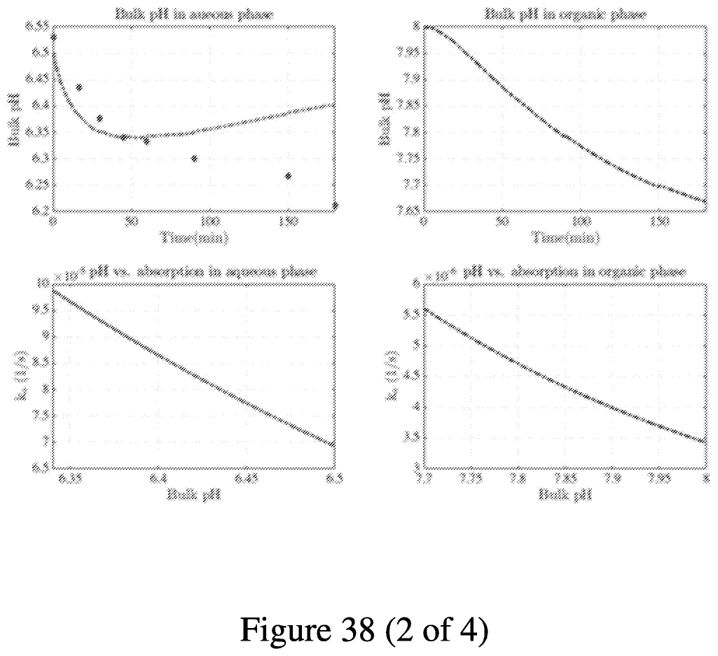

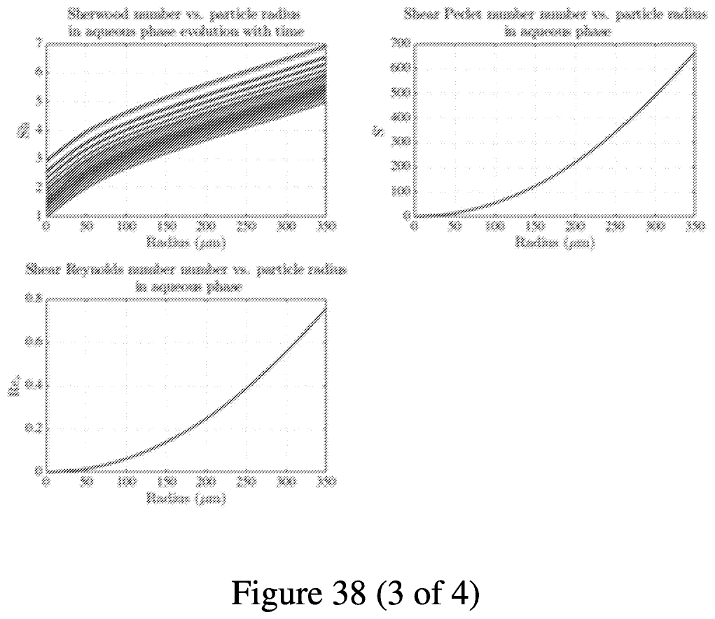

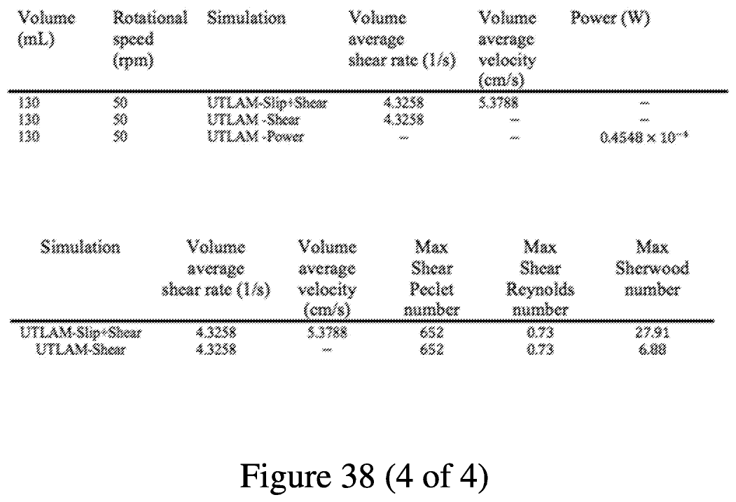

The simulations show that there is a mismatch between the experiment and the simulation when unidirectional flux is assumed from donor to receiver phase. After bi-directional flux was added to the code and the predicted mass at the test end point was more accurate (there were still solid particles at the end point), but simulation as a whole were still very inaccurate (see FIG. 38).

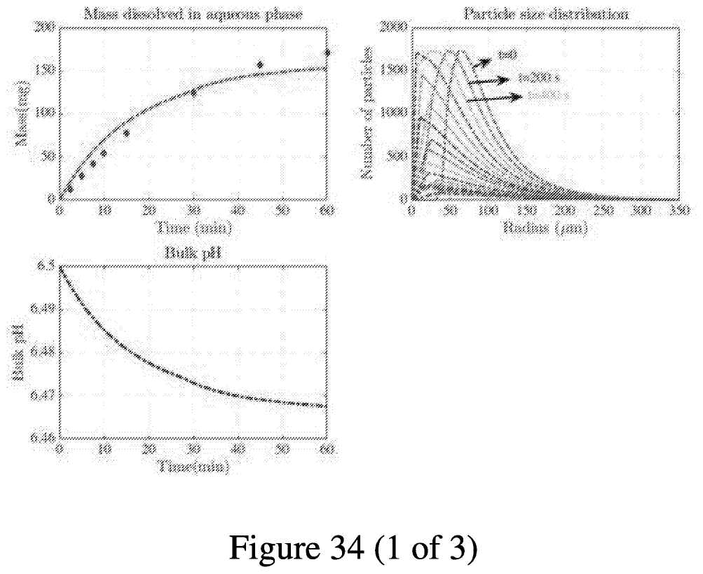

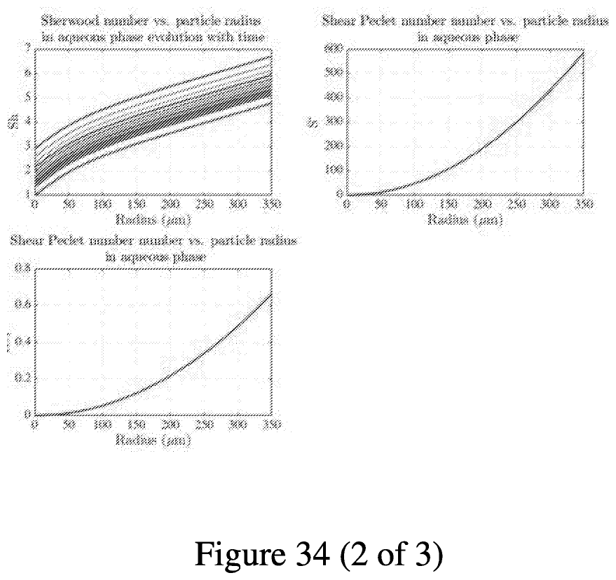

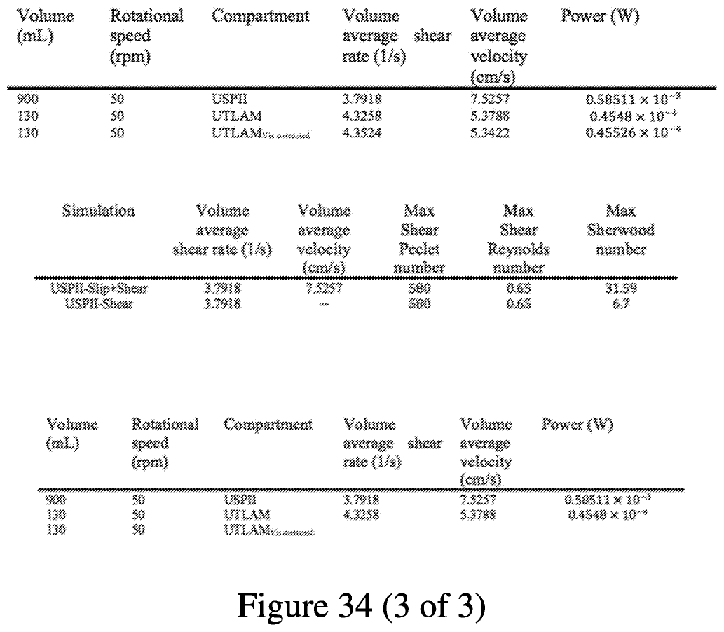

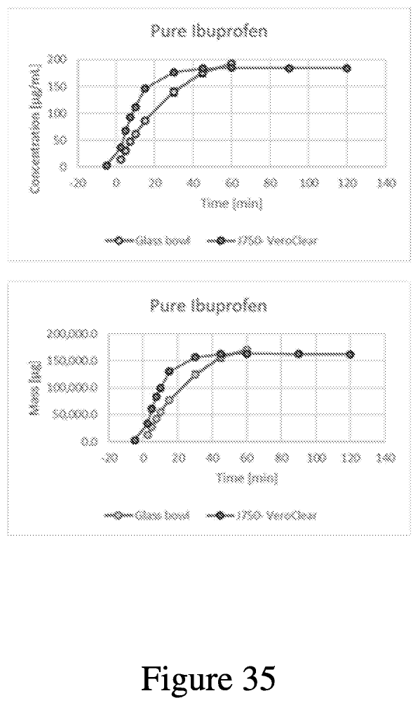

The data disclosed herein establish that, not only does the resin partition significant quantities of ibuprofen, but that the process water used to warm the device was acting like a drug sink. The USP 2 vessel was modeled in CAD and COMSOL and the MATLAB simulation of the dissolution experiment was conducted for both the compendial vessel and 3D printed vessel (FIG. 34-35).

### Example 10

Ultra Thin, Large Area Membrane (UTLAM) In Vivo Relevant Dissolution Experiments

Once the UTLAM is fabricated in a partition resistant material, experiments with pH 4.95 and 5.5 in low buffer capacity phosphate buffer are performed to more closely mimic the pH conditions in the duodenum and jejunum. The impeller speed is reduced to reduce the bulk shear rate so that the Shear Peclet and Shear Reynolds numbers are more consistent with in vivo values. With the decrease in pH, it is expected that the absorption rate will increase significantly. This would indicate that the UTLAM device will still perform within expectations and prove useful as in in vitro model of in vivo drug absorption behavior.

### Example 11

Derivation of Log D formula for monoprotic acid

$$\begin{matrix}[ {R}_{A q u e o u s}^{non \text{-} i o n i z e d} ] = [ R ] & ( S1 ) \\ [ {R}_{A q u e o u s}^{i o n i z e d} ] = [ R H ] & ( S2 ) \\ [ RH ] {↔}_{ka} [ R ] + [ H ] ; ka = \frac{[ R ] [ H ]}{[ R H ]} & ( S3 )\end{matrix}$$

Write the ionization reaction and define the reaction rate constant.

$$\begin{matrix}[ R H ] = \frac{[ R ] [ H ]}{k a} & ( S4 ) \\ [ {R}_{T} ] = \sum [ {R}_{i} ] ; [ {R}_{T} ] = [ R H ] + [ R ] & ( S5 )\end{matrix}$$

The total amount of drug in the mass balance is the summation of all the forms of the drug present in the acid-base reaction.

$$[ {R}_{T} ] = \frac{[ R ] [ H ]}{k a} + [ R ] \qquad ( S6 )$$

Using the rate constant to solve for the ionized form of the drug, establish the total amount of drug in the system as a function of the non-ionized form.

$$[ {R}_{T} ] = ( \frac{[ H ]}{k a} + 1 ) [ R ] \qquad ( S7 )$$

The partition coefficient is defined as the ratio of non-ionized drug in the non-aqueous phase and the non-ionized drug in the aqueous phase.

$$K = \frac{[ {R}_{Octanol}^{non \text{-} ionized} ]}{[ {R}_{A q u e o u s}^{non \text{-} ionized} ]} \qquad ( S8 )$$

The distribution coefficient is defined as the ratio non-ionized drug in the non-aqueous phase to the sum of the ionized and non-ionized form of the drug in the aqueous.

$$D = \frac{[ {R}_{Octanol}^{non \text{-} ionized} ]}{[ {R}_{Aqueou s}^{ionized} ] + [ {R}_{A q u e o u s}^{non \text{-} ionized} ]} \qquad ( S9 )$$

Rearranging the partition coefficient equation:

$$\begin{matrix}[ {R}_{Octanol}^{non \text{-} i o n i z e d} ] = K [ {R}_{A q u e o u s}^{non \text{-} i o n i z e d} ] & ( S10 ) \\ [ {R}_{non \text{-} ionized} ] = ( \frac{k a}{[ H ]} + 1 ) [ {R}_{T} ] & ( S11 )\end{matrix}$$

Substitute all the values into the distribution coefficient formula.

$$D = \frac{K [ {R}_{T} ] ( \frac{k a}{[ H ]} + 1 )}{\frac{[ R ] [ H ]}{ka} + ( \frac{k a}{[ H ]} + 1 ) [ {R}_{T} ]} \qquad ( S12 )$$

Factor out the R_{T} term and divide it out

$$D = \frac{K [ {R}_{T} ] ( \frac{k a}{[ H ]} + 1 )}{\frac{[ H ]}{ka} ( \frac{k a}{[ H ]} + 1 ) [ {R}_{T} ] + ( \frac{k a}{[ H ]} + 1 ) [ {R}_{T} ]} \qquad ( S13 )$$

Divide out the (ka/[H]+1) term

$$D = \frac{K ( \frac{k a}{[ H ]} + 1 )}{\frac{[ H ]}{ka} ( \frac{k a}{[ H ]} + 1 ) + ( \frac{k a}{[ H ]} + 1 )} \qquad ( S14 )$$

We now know the distribution partition coefficient as a function of the hydrogen ion concentration. To make this more useful we convert ka/[H] into pH and pKa.

$$\begin{matrix}D = \frac{K}{( \frac{[ H ]}{k a} + 1 )} & ( S15 ) \\ pKa = - {log}_{10} {K}_{a} & ( S16 ) \\ pH = - {log}_{10} [ H ] & ( S17 ) \\ 1 {0}^{- p K a} = {K}_{a} & ( S18 ) \\ 1 {0}^{- p H} = [ H ] & ( S19 ) \\ D = \frac{K}{( \frac{[ 1 {0}^{- p H} ]}{1 {0}^{- p K a}} + 1 )} & ( S20 )\end{matrix}$$

Take the logarithm of both sides of the equation

$$log D = \frac{log K}{log ( \frac{1 {0}^{- p H}}{1 {0}^{- p K a}} + 1 )} \qquad ( S21 )$$

Simplify the logarithm and obtain the pH distributed partition coefficient

$$log D = \frac{log K}{log ( {10}^{ pH - pKa} + 1 )} \qquad ( S22 )$$

Derivation of Levich rotating disk aqueous boundary layer thickness

$$\frac{k d}{D} = 0.62 * {( \frac{{d}^{2} ω}{v} )}^{1 / 2} * {( \frac{v}{D} )}^{1 / 3} \qquad ( S23 )$$

Dividing by d

$$\begin{matrix}d = 0. 62 \frac{d {ω}^{1 / 2}}{{v}^{1 / 2}} * \frac{{v}^{1 / 3}}{{D}^{1 / 3}} = 0.62 \frac{d {ω}^{1 / 2}}{{v}^{3 / 6}} * \frac{{v}^{2 / 6}}{{D}^{1 / 3}} = 0.62 \frac{d {ω}^{1 / 2}}{{v}^{1 / 6}} * \frac{1}{{D}^{1 / 3}} & ( S24 ) \\ \begin{matrix} \frac{k d}{D} = 0.62 \frac{d {ω}^{1 / 2}}{{v}^{1 / 6}} * \frac{1}{{D}^{1 / 3}} \\ = 0.62 * {v}^{- 1 / 6} * {D}^{- 1 / 3} * d * {ω}^{1 / 2}\end{matrix} & ( S25 ) \\ Since \text{:} \frac{k}{D} = 0.62 * {v}^{- 1 / 6} * {D}^{- 1 / 3} * {ω}^{1 / 2} = [ \frac{1}{meters} ] & ( S26 )\end{matrix}$$

Therefore:

$${h}_{a q} = 1.61 * {( \frac{v}{100 \times ρ} )}^{1 / 6} * {D}^{1 / 3} * {ω}^{- 1 / 2} \qquad ( S27 )$$

### Where:

ν=is the viscosity in centipoise
ρ=fluid density g/cm³
h_{aq}=is the boundary layer thickness in centimeters
ω=(2*π*Rotations per min)/60
D=diffusion coefficient in cm²/s
Derivation of the Permeability equation using Permeability Layer Theory
P_{eff}=effective membrane permeability
P_{aq}=Permeability of the aqueous boundary layer
P_{PDMS}=Permeability of the membrane
V_{receiver}=drug receiving phase volume
Aₘ=area of membrane for transport
h_{aq}=Levich boundary layer thickness
D_{ag}=diffusion coefficient of the drug through the aqueous medium
hₘ=thickness of membrane
D_{PDMS}=diffusion coefficient of the drug through PDMS membrane
K_{PDMS}=partition coefficient of the drug in PDMS

Under sink conditions:

C_{sol limit}=S=Drug solubility & C_{receiver}=0

$${J}_{eff} = {P}_{eff} * Δ C = {P}_{eff} * ( {C}_{sol limit} - {C}_{receiver} ) = \frac{1}{\frac{1}{{P}_{aq}} + \frac{1}{{P}_{P D M S}}} * S \qquad ( S28 )$$

Where m=slope of the concentration versus time curve from experiment with units [μg/(mL*s)]

$${J}_{experimental} = \frac{1}{{A}_{m}} * m * {V}_{receiver} \qquad ( S29 )$$

Substituting in permeability components

$$\frac{1}{{A}_{m}} * m * {V}_{receiver} = \frac{1}{\frac{{h}_{aq}}{{D}_{aq}} + \frac{{h}_{m}}{{K}_{P D M S} {D}_{P D M S}}} * S \qquad ( S30 )$$

Divide by the solubility

$$( \frac{m * {V}_{receiver}}{{A}_{m} * S} ) = \frac{1}{\frac{{h}_{aq}}{{D}_{aq}} + \frac{{h}_{m}}{{K}_{P D M S} {D}_{P D M S}}} \qquad ( S31 )$$

Simplify

$$\frac{m * {V}_{receiver}}{{A}_{m} * S} = \frac{{D}_{a q}}{{h}_{a q}} + \frac{{K}_{PDMS} {D}_{P D M S}}{{h}_{m}} \qquad ( S31 )$$

Because:

$$\begin{matrix}{P}_{eff} = {P}_{experimental} = \frac{1}{\frac{1}{{P}_{aq}} + \frac{1}{{P}_{P D M S}}} & ( S32 ) \\ \frac{1}{{P}_{a q}} + \frac{1}{{P}_{P D M S}} = \frac{1}{{P}_{experimental}} & ( S33 )\end{matrix}$$

Isolating experimental and aqueous diffusion components from the membrane components:

$$\begin{matrix}\frac{1}{{P}_{experimental}} - \frac{1}{{P}_{a q}} = \frac{1}{{P}_{P D M S}} & ( S34 ) \\ \frac{{A}_{m} * S}{m * {V}_{receiver}} - \frac{{h}_{a q}}{{D}_{a q}} = \frac{{h}_{m}}{{K}_{PDMS} {D}_{P D M S}} & ( S35 ) \\ {[ \frac{{A}_{m} * S}{m * {V}_{receiver}} - \frac{{h}_{a q}}{{D}_{a q}} ]}^{- 1} = {[ \frac{{h}_{m}}{{K}_{PDMS} {D}_{P D M S}} ]}^{- 1} = {[ {P}_{P D M S} ]}^{- 1} & ( S36 )\end{matrix}$$

Derivation of the Permeability Equation Using Crank's Approach to Calculating Diffusion Coefficient

Q_{T}=[Mass/Area] flowing through the membrane
D=diffusion coefficient of the transporting molecule
C₂=the surface concentration at the inner surface of the membrane
h=thickness of the membrane
t=time
P=permeability of the molecule through the membrane
m_{dC/dt}=the slope of the pseudo-steady state transport region on the concentration versus time plot
V_{receiver}=aqueous volume in the receiver compartment
K_{PDMS}=the partition coefficient of the molecule in PDMS
C_{aq total}=total aqueous concentration of the molecule in the bulk donor phase

$$\begin{matrix}{Q}_{T} = \frac{D {C}_{2}}{h} ( t - \frac{{h}^{2}}{6 D} ) & ( S37 ) \\ {D}_{1} = \frac{{Q}_{T} \times h}{{c}_{2}} & ( S38 ) \\ {D}_{2} = \frac{{h}^{2}}{6 t} & ( S39 ) \\ {D}_{1} = {D}_{2} & ( S40 )\end{matrix}$$

First we measure the slope of the concentration versus time curve in the pseudo steady state region and convert it into mass per area.

$${Q}_{T} = \frac{{m}_{d C / d t} \times {V}_{receiver}}{{A}_{m e m b r a n e}} \qquad ( S41 )$$

Then we calculate the concentration at the inner surface of the membrane, which is the total concentration in the aqueous donor phase multiplied by the partition coefficient of the molecule in the material.

C₂=K_{PDMS}×C_{aq Total} (S42)

The definition of permeability:

$$P = \frac{D}{h} \qquad ( S43 )$$

Rearrange D₁:

$$\begin{matrix}{D}_{1} = \frac{{Q}_{T} \times h}{{c}_{2}} & ( S44 ) \\ \frac{{D}_{1}}{h} = \frac{{Q}_{T}}{{C}_{2}} & ( S45 )\end{matrix}$$

Fully substituting all variables for experimental measurements:

$${P}_{P D M S} = \frac{{D}_{1}}{h} = \frac{\frac{{m}_{dC / d} \times {V}_{receiver}}{{A}_{m e m b r a n e}}}{{K}_{P D M S} \times {C}_{aq Total}} \qquad ( S46 )$$

As a check: Since D₁=D₂ we can compare the measured time to steady state from the rotating membrane diffusion cell experiment with the time predicted in D₂.

$$\begin{matrix}{D}_{2} = \frac{{h}^{2}}{6 t} & ( S47 ) \\ t = \frac{{h}^{2}}{6 {D}_{2}} \to \frac{{h}^{2}}{6 {D}_{1}} & ( S48 )\end{matrix}$$

### REFERENCES

1. D. M. Mudie, G. L. Amidon, G. E. Amidon, Physiological Parameters for Oral Delivery and in Vitro Testing, Mol. Pharm., 7 (2010) 1388-1405.
2. D. M. Mudie, K. Murray, C. L. Hoad, S. E. Pritchard, M. C. Garnett, G. L. Amidon, P. A. Gowland, R. C. Spiller, G. E. Amidon, L. Marciani, Quantification of Gastrointestinal Liquid Volumes and Distribution Following a 240 mL Dose of Water in the Fasted State, Mol. Pharm., 11 (2014) 3039-3047.
3. B. J. Krieg, S. M. Taghavi, G. L. Amidon, G. E. Amidon, In Vivo Predictive Dissolution: Comparing the Effect of Bicarbonate and Phosphate Buffer on the Dissolution of Weak Acids and Weak Bases, J. Pharm. Sci., 104 (2015) 2894-2904.
4. B. J. Krieg, S. M. Taghavi, G. L. Amidon, G. E. Amidon, In Vivo Predictive Dissolution: Transport Analysis of the CO2, Bicarbonate In Vivo Buffer System, J. Pharm. Sci., 103 (2014) 3473-3490.
5. G. E. Amidon, Rotating Membrane Diffusion Studies of Micellar and Suspension Systems, in: Pharmaceutical Chemistry, University of Michigan, Ann Arbor, 1979, pp. 183.
6. D. M. Mudie, Y. Shi, H. L. Ping, P. Gao, G. L. Amidon, G. E. Amidon, Mechanistic analysis of solute transport in an in vitro physiological two-phase dissolution apparatus, Biopharm. Drug Dispos., 33 (2012) 378-402.
7. Y. Shi, P. Gao, Y. C. Gong, H. L. Ping, Application of a Biphasic Test for Characterization of In Vitro Drug Release of Immediate Release Formulations of Celecoxib and Its Relevance to In Vivo Absorption, Mol. Pharm., 7 (2010) 1458-1465.
8. Y. Shi, B. Erickson, A. Jayasankar, L. J. Lu, K. Marsh, R. Menon, P. Gao, Assessing Supersaturation and Its Impact on In Vivo Bioavailability of a Low-Solubility Compound ABT-072 With a Dual pH, Two-Phase Dissolution Method, J. Pharm. Sci., 105 (2016) 2886-2895.
9. P. Gao, Y. Shi, Characterization of Supersaturatable Formulations for Improved Absorption of Poorly Soluble Drugs, Aaps Journal, 14 (2012) 703-713.
10. K. Locher, J. M. Borghardt, K. J. Frank, C. Kloft, K. G. Wagner, Evolution of a mini-scale biphasic dissolution model: Impact of model parameters on partitioning of dissolved API and modelling of in vivo-relevant kinetics, Eur. J. Pharm. Biopharm., 105 (2016) 166-175.
11. K. J. Frank, K. Locher, D. E. Zecevic, J. Fleth, K. G. Wagner, In vivo predictive mini-scale dissolution for weak bases: Advantages of pH-shift in combination with an absorptive compartment, Eur. J. Pharm. Sci., 61 (2014) 32-39.
12. P. C. Stein, M. di Cagno, A. Bauer-Brandl, A Novel Method for the Investigation of Liquid/Liquid Distribution Coefficients and Interface Permeabilities Applied to the Water-Octanol-Drug System, Pharmaceutical Research, 28 (2011) 2140-2146.
13. R. Takano, M. Kataoka, S. Yamashita, Integrating drug permeability with dissolution profile to develop IVIVC, Biopharm. Drug Dispos., 33 (2012) 354-365.
14. M. Kataoka, K. Sugano, C. da Costa Mathews, J. W. Wong, K. L. Jones, Y. Masaoka, S. Sakuma, S. Yamashita, Application of Dissolution/Permeation System for Evaluation of Formulation Effect on Oral Absorption of Poorly Water-Soluble Drugs in Drug Development, Pharmaceutical Research, 29 (2012) 1485-1494.
15. M. Kataoka, K. Yano, Y. Hamatsu, Y. Masaoka, S. Sakuma, S. Yamashita, Assessment of absorption potential of poorly water-soluble drugs by using the dissolution/permeation system, Eur. J. Pharm. Biopharm., 85 (2013) 1317-1324.
16. Y. Miyaji, Y. Fujii, S. Takeyama, Y. Kawai, M. Kataoka, M. Takahashi, S. Yamashita, Advantage of the Dissolution/Permeation System for Estimating Oral Absorption of Drug Candidates in the Drug Discovery Stage, Mol. Pharm., 13 (2016) 1564-1574.
17. M. Kataoka, Y. Masaoka, Y. Yamazaki, T. Sakane, H. Sezaki, S. Yamashita, In Vitro System to Evaluate Oral Absorption of Poorly Water-Soluble Drugs: Simultaneous Analysis on Dissolution and Permeation of Drugs, Pharmaceutical Research, 20 (2003) 1674-1680.
18. K. Yano, Y. Masaoka, M. Kataoka, S. Sakuma, S. Yamashita, Mechanisms of Membrane Transport of Poorly Soluble Drugs: Role of Micelles in Oral Absorption Processes, J. Pharm. Sci., 99 (2010) 1336-1345.
19. M. Kataoka, S. Itsubata, Y. Masaoka, S. Sakuma, S. Yamashita, In Vitro Dissolution/Permeation System to Predict the Oral Absorption of Poorly Water-Soluble Drugs: Effect of Food and Dose Strength on It, Biological and Pharmaceutical Bulletin, 34 (2011) 401-407.
20. S. T. Buckley, S. M. Fischer, G. Fricker, M. Brandi, In vitro models to evaluate the permeability of poorly soluble drug entities: Challenges and perspectives, Eur. J. Pharm. Sci., 45 (2012) 235-250.
21. A. Adson, P. S. Burton, T. J. Raub, C. L. Barsuhn, K. L. Audus, N. F. H. Ho, Passive Diffusion of Weak Organic Electrolytes across Caco-2 Cell Monolayers: Uncoupling the Contributions of Hydrodynamic, Transcellular, and Paracellular Barriers, J. Pharm. Sci., 84 (1995) 1197-1204.
22. M. Kansy, F. Senner, K. Gubernator, Physicochemical High Throughput Screening: Parallel Artificial Membrane Permeation Assay in the Description of Passive Absorption Processes, Journal of Medicinal Chemistry, 41 (1998) 1007-1010.
23. L. Di, E. H. Kerns, K. Fan, O. J. McConnell, G. T. Carter, High throughput artificial membrane permeability assay for blood-brain barrier, European Journal of Medicinal Chemistry, 38 (2003) 223-232.
24. G. Ottaviani, S. Martel, P.-A. Carrupt, Parallel Artificial Membrane Permeability Assay: A New Membrane for the Fast Prediction of Passive Human Skin Permeability, Journal of Medicinal Chemistry, 49 (2006) 3948-3954.
25. J. Mensch, A. Melis, C. Mackie, G. Verreck, M. E. Brewster, P. Augustijns, Evaluation of various PAMPA models to identify the most discriminating method for the prediction of BBB permeability, Eur. J. Pharm. Biopharm., 74 (2010) 495-502.
26. P. R. Seo, Z. S. Teksin, J. P. Y. Kao, J. E. Polli, Lipid composition effect on permeability across PAMPA, Eur. J. Pharm. Sci., 29 (2006) 259-268.
27. E. H. Kerns, L. Di, S. Petusky, M. Farris, R. Ley, P. Jupp, Combined Application of Parallel Artificial Membrane Permeability Assay and Caco-2 Permeability Assays in Drug Discovery, J. Pharm. Sci., 93 (2004) 1440-1453.
28. A. Avdeef, P. E. Nielsen, 0. Tsinman, PAMPA—a drug absorption in vitro model, Eur. J. Pharm. Sci., 22 (2004) 365-374.
29. J. Siepmann, A. Gopferich, Mathematical Modeling of Bioerodible, polymeric drug delivery systems, Adv. Drug Deliv. Rev., 48 (2001) 229-247.
30. L. H. Sperling, Introduction to Physical Polymer Science, 4th ed., John Wiley & Sons, Hoboken, N.J., 2006.
31. K. Efimenko, W. E. Wallace, J. Genzer, Surface modification of Sylgard-184 poly(dimethyl siloxane) networks by ultraviolet and ultraviolet/ozone treatment, J. Colloid Interface Sci., 254 (2002) 306-315.
32. D. M. Dattelbaum, J. D. Jensen, A. M. Schwendt, E. M. Kober, M. W. Lewis, R. Menikoff, A novel method for static equation-of state-development: Equation of state of a cross-linked poly(dimethylsiloxane) (PDMS) network to 10 GPa, J. Chem. Phys., 122 (2005) 12.
33. E. R. Garrett, P. B. Chemburkar, Evaluation Control And Prediction Of Drug Diffusion Through Polymeric Membranes 0.3. Diffusion Of Barbiturates Phenylalkylamines Dextromethorphan Progesterone And Other Drugs, J. Pharm. Sci., 57 (1968) 1401-+.
34. D. W. Gidley, H. G. Peng, R. S. Vallery, Positron annihilation as a method to characterize porous materials, in: Annual Review of Materials Research, Annual Reviews, Palo Alto, 2006, pp. 49-79.
35. R. S. Vallery, P. W. Zitzewitz, D. W. Gidley, Resolution of the orthopositronium-lifetime puzzle, Physical Review Letters, 90 (2003) 4.
36. Z. Kajcsos, L. Liszkay, G. Duplatre, L. Varga, L. Lohonyai, F. Paszti, E. Szilagyi, K. Lazar, E. Kotai, G. Pal-Borbely, H. K. Beyer, P. Caullet, J. Patarin, M. E. Azenha, P. M. Gordo, C. L. Gil, A. P. de Lima, M. F. F. Margues, Positronium trapping in porous solids: Means and limitations for structural studies, Acta Phys. Pol. A, 107 (2005) 729-737.
37. M. Eldrup, D. Lightbody, J. N. Sherwood, The Temperature-Dependence Of Positron Lifetimes In Solid Pivalic Acid, Chem. Phys., 63 (1981) 51-58.
38. D. Fragiadakis, P. Pissis, L. Bokobza, Glass transition and molecular dynamics in poly (dimethylsiloxane)/silica nanocomposites, Polymer, 46 (2005) 6001-6008.
39. A. Dahan, 0. Wolk, Y. H. Kim, C. Ramachandran, G. M. Crippen, T. Takagi, M. Bermejo, G. L. Amidon, Purely in Silico BCS Classification: Science Based Quality Standards for the World's Drugs, Mol. Pharm., 10 (2013) 4378-4390.
40. J. G. Wagner, A. J. Sedman, Quantitaton of rate of gastrointestinal and buccal absorption of acidic and basic drugs based on extraction theory, Journal of Pharmacokinetics and Biopharmaceutics, 1 (1973) 23-50.
41. S. Winiwarter, N. M. Bonham, F. Ax, A. Hallberg, H. Lennernas, A. Karlen, Correlation of human jejunal permeability (in vivo) of drugs with experimentally and theoretically derived parameters. A multivariate data analysis approach, Journal of Medicinal Chemistry, 41 (1998) 4939-4949.
42. J. Crank, The mathematics of diffusion, Clarendon Press, Oxford, [Eng], 1975.
43. R. A. Siegel, A laplace transform technique for calculating diffusion time lags, Journal of Membrane Science, 26 (1986) 251-262.
44. D. Winne, Shift of pH-absorption curves, Journal of Pharmacokinetics and Biopharmaceutics, 5 (1977) 53-94.
45. W. Hayduk, H. Laudie, Prediction of Diffusion Coefficients for nonelectrolytes in dilute aqueous solutions, A.I.Ch.E. Journal, 20 (1974) 610-615.
46. Z. Wang, Polydimethylsiloxane Mechanical Properties Measured by Macroscopic Compression and Nanoindentation Techniques, in: Mechanical Engineering, University of South Florida, 2011, pp. 79.
47. J. S. Gao, D. Z. Guo, S. Santhanam, G. K. Fedder, Material Characterization and Transfer of Large-Area Ultra-Thin Polydimethylsiloxane Membranes, J. Microelectromech. Syst., 24 (2015) 2170-2177.
48. J. M. K. Ng, I. Gitlin, A. D. Stroock, G. M. Whitesides, Components for integrated poly(dimethylsiloxane) microfluidic systems, ELECTROPHORESIS, 23 (2002) 3461-3473.
49. Sinko, P. D.; Gidley, D.; Vallery, R.; Lamoureux, A.; Amidon, G. L.; Amidon, G. E. In Vitro Characterization of the Biomimetic Properties of Poly(dimethylsiloxane) To Simulate Oral Drug Absorption. Mol. Pharm. 2017, 14, (12), 4661-4674.
50. Hens, B.; Sinko, P.; Job, N.; Dean, M.; Al-Gousous, J.; Salehi, N.; Ziff, R. M.; Tsume, Y.; Bermejo, M.; Paixao, P.; Brasseur, J. G.; Yu, A.; Talattof, A.; Benninghoff, G.; Langguth, P.; Lennernas, H.; Hasler, W. L.; Marciani, L.; Dickens, J.; Shedden, K.; Sun, D.; Amidon, G. E.; Amidon, G. L. Formulation predictive dissolution (fPD) testing to advance oral drug product development: An introduction to the US FDA funded ‘21st Century BA/BE’ project. Int. J. Pharm. 2018, 548, (1), 120-127.
51. Hens, B.; Tsume, Y.; Bermejo, M.; Paixao, P.; Koenigsknecht, M. J.; Baker, J. R.; Hasler, W. L.; Lionberger, R.; Fan, J.; Dickens, J.; Shedden, K.; Wen, B.; Wysocki, J.; Loebenberg, R.; Lee, A.; Frances, A.; Amidon, G.; Yu, A.; Benninghoff, G.; Salehi, N.; Talattof, A.; Sun, D.; Amidon, G. L. Low Buffer Capacity and Alternating Motility along the Human Gastrointestinal Tract: Implications for in Vivo Dissolution and Absorption of Ionizable Drugs. Mol. Pharm. 2017, 14, (12), 4281-4294.
52. Cohen, J. L.; Hubert, B. B.; Leeson, L. J.; Rhodes, C. T.; Robinson, J. R.; Roseman, T. J.; Shefter, E. The Development of USP Dissolution and Drug Release Standards. Pharmaceutical Research 1990, 7, (10), 983-987.
53. Vertzoni, M.; Dressman, J.; Butler, J.; Hempenstall, J.; Reppas, C. Simulation of fasting gastric conditions and its importance for the in vivo dissolution of lipophilic compounds. Eur. J. Pharm. Biopharm. 2005, 60, (3), 413-417.
54. Kostewicz, E. S.; Abrahamsson, B.; Brewster, M.; Brouwers, J.; Butler, J.; Carlert, S.; Dickinson, P. A.; Dressman, J.; Holm, R.; Klein, S.; Mann, J.; McAllister, M.; Minekus, M.; Muenster, U.; Miillertz, A.; Verwei, M.; Vertzoni, M.; Weitschies, W.; Augustijns, P. In vitro models for the prediction of in vivo performance of oral dosage forms. Eur. J. Pharm. Sci. 2014, 57, 342-366.
55. Fuchs, A.; Leigh, M.; Kloefer, B.; Dressman, J. B. Advances in the design of fasted state simulating intestinal fluids: FaSSIF-V3. Eur. J. Pharm. Biopharm. 2015, 94, 229-240.
56. Bergstrom, C. A. S.; Holm, R.; Jorgensen, S. A.; Andersson, S. B. E.; Artursson, P.; Beato, S.; Borde, A.; Box, K.; Brewster, M.; Dressman, J.; Feng, K.-I.; Halbert, G.; Kostewicz, E.; McAllister, M.; Muenster, U.; Thinnes, J.; Taylor, R.; Mullertz, A. Early pharmaceutical profiling to predict oral drug absorption: Current status and unmet needs. Eur. J. Pharm. Sci. 2014, 57, 173-199.
57. Markopoulos, C.; Andreas, C. J.; Vertzoni, M.; Dressman, J.; Reppas, C. In-vitro simulation of luminal conditions for evaluation of performance of oral drug products: Choosing the appropriate test media. Eur. J. Pharm. Biopharm. 2015, 93, 173-182.
58. Augustijns, P.; Wuyts, B.; Hens, B.; Annaert, P.; Butler, J.; Brouwers, J. A review of drug solubility in human intestinal fluids: Implications for the prediction of oral absorption. Eur. J. Pharm. Sci. 2014, 57, 322-332.
59. Lennernäs, H.; Aarons, L.; Augustijns, P.; Beato, S.; Bolger, M.; Box, K.; Brewster, M.; Butler, J.; Dressman, J.; Holm, R.; Julia Frank, K.; Kendall, R.; Langguth, P.; Sydor, J.; Lindahl, A.; McAllister, M.; Muenster, U.; Müllertz, A.; Ojala, K.; Pepin, X.; Reppas, C.; Rostami-Hodjegan, A.; Verwei, M.; Weitschies, W.; Wilson, C.; Karlsson, C.; Abrahamsson, B. Oral biopharmaceutics tools—Time for a new initiative—An introduction to the IMI project OrBiTo. Eur. J. Pharm. Sci. 2014, 57, 292-299.
60. Klein, S. The Use of Biorelevant Dissolution Media to Forecast the In Vivo Performance of a Drug. The AAPS Journal 2010, 12, (3), 397-406.
61. Garbacz, G.; Wedemeyer, R.-S.; Nagel, S.; Giessmann, T.; Mönnikes, H.; Wilson, C. G.; Siegmund, W.; Weitschies, W. Irregular absorption profiles observed from diclofenac extended release tablets can be predicted using a dissolution test apparatus that mimics in vivo physical stresses. Eur. J. Pharm. Biopharm. 2008, 70, (2), 421-428.
62. Dressman, J. B.; Amidon, G. L.; Reppas, C.; Shah, V. P. Dissolution Testing as a Prognostic Tool for Oral Drug Absorption: Immediate Release Dosage Forms. Pharmaceutical Research 1998, 15, (1), 11-22.
63. Behafarid, F., Brasseur, J. G., Vijayakumar, G., Jayaraman, B., Wang, Y., Computational Studies of Drug Release, Transport and Absorption in the Human Intestines. Bull. Amer. Phys. Soc., 2016.
64. Behafarid, F., Vijayakumar, G., Brasseur, J. G., The Interplay between Pharmaceutical Dissolution and Absorption In the Human Gut studied with Computer Simulation. American Association of Pharmaceutical Scientists (AAPS) Annual Meeting Denver, Colo., 2016.
65. Brasseur, J. G., Behafarid, F., Wang, Y., Mudie, D., Amidon, G., Hydrodynamic Influences on Drug Dissolution and Absorption In Vitro and In Vivo, quantified with Mathematical Models and Computer Simulation. 6th Pharmaceutical Sciences World Congress (FIP PSWC 2017), 2017.
66. Melchels, F. P. W.; Feijen, J.; Grijpma, D. W. A review on stereolithography and its applications in biomedical engineering. Biomaterials 2010, 31, (24), 6121-6130.
67. Pham, D. T.; Gault, R. S. A comparison of rapid prototyping technologies. Int. J. Mach. Tools Manuf. 1998, 38, (10-11), 1257-1287.
68. Gross, B. C.; Erkal, J. L.; Lockwood, S. Y.; Chen, C.; Spence, D. M. Evaluation of 3D Printing and Its Potential Impact on Biotechnology and the Chemical Sciences. Analytical Chemistry 2014, 86, (7), 3240-3253.
69. Wang, J.; Goyanes, A.; Gaisford, S.; Basit, A. W. Stereolithographic (SLA) 3D printing of oral modified-release dosage forms. Int. J. Pharm. 2016, 503, (1), 207-212.
70. Goyanes, A.; Kobayashi, M.; Martinez-Pacheco, R.; Gaisford, S.; Basit, A. W. Fused-filament 3D printing of drug products: Microstructure analysis and drug release characteristics of PVA-based caplets. Int. J. Pharm. 2016, 514, (1), 290-295.
71. Alhijjaj, M.; Belton, P.; Qi, S. An investigation into the use of polymer blends to improve the printability of and regulate drug release from pharmaceutical solid dispersions prepared via fused deposition modeling (FDM) 3D printing. Eur. J. Pharm. Biopharm. 2016, 108, (Supplement C), 111-125.
72. Melocchi, A.; Parietti, F.; Maroni, A.; Foppoli, A.; Gazzaniga, A.; Zema, L. Hot-melt extruded filaments based on pharmaceutical grade polymers for 3D printing by fused deposition modeling. Int. J. Pharm. 2016, 509, (1), 255-263.
73. Sadia, M.; Sonicka, A.; Arafat, B.; Isreb, A.; Ahmed, W.; Kelarakis, A.; Alhnan, M. A. Adaptation of pharmaceutical excipients to FDM 3D printing for the fabrication of patient-tailored immediate release tablets. Int. J. Pharm. 2016, 513, (1), 659-668.
74. Pietrzak, K.; Isreb, A.; Alhnan, M. A. A flexible-dose dispenser for immediate and extended release 3D printed tablets. Eur. J. Pharm. Biopharm. 2015, 96, (Supplement C), 380-387.
75. Goyanes, A.; Chang, H.; Sedough, D.; Hatton, G. B.; Wang, J.; Buanz, A.; Gaisford, S.; Basit, A. W. Fabrication of controlled-release budesonide tablets via desktop (FDM) 3D printing. Int. J. Pharm. 2015, 496, (2), 414-420.
76. Goyanes, A.; Buanz, A. B. M.; Basit, A. W.; Gaisford, S. Fused-filament 3D printing (3DP) for fabrication of tablets. Int. J. Pharm. 2014, 476, (1), 88-92.
77. Martinez, P. R.; Goyanes, A.; Basit, A. W.; Gaisford, S. Fabrication of drug-loaded hydrogels with stereolithographic 3D printing. Int. J. Pharm. 2017, 532, (1), 313-317.
78. Sharma, R. P.; Green, P. F. Role of “Hard” and “Soft” Confinement on Polymer Dynamics at the Nanoscale. ACS Macro Letters 2017, 6, (9), 908-914.
79. Brown, H. R.; Char, K.; Deline, V. R.; Green, P. F. Effects of a diblock copolymer on adhesion between immiscible polymers. 1. Polystyrene (PS)-PMMA copolymer between PS and PMMA. Macromolecules 1993, 26, (16), 4155-4163.
80. Kukura, J.; Baxter, J. L.; Muzzio, F. J. Shear distribution and variability in the USP Apparatus 2 under turbulent conditions. Int. J. Pharm. 2004, 279, (1), 9-17.
81. Ameur, H.; Bouzit, M. 3D hydrodynamics and shear rates' variability in the United States Pharmacopeia Paddle Dissolution Apparatus. Int. J. Pharm. 2013, 452, (1), 42-51.
82. Bai, G.; Wang, Y.; Armenante, P. M. Velocity profiles and shear strain rate variability in the USP Dissolution Testing Apparatus 2 at different impeller agitation speeds. Int. J. Pharm. 2011, 403, (1), 1-14.
83. Wang, Y.; Armenante, P. M. A Novel Off-Center Paddle Impeller (OPI) Dissolution Testing System for Reproducible Dissolution Testing of Solid Dosage Forms. J. Pharm. Sci. 2012, 101, (2), 746-760.
84. Bai, G.; Armenante, P. M. Hydrodynamic, mass transfer, and dissolution effects induced by tablet location during dissolution testing. J. Pharm. Sci. 2009, 98, (4), 1511-1531.
95. Rieger, F.; Jirout, T.; Ceres, D.; Seichter, P., Effect of Impeller Shape on Solid Particle Suspension. In Chemical and Process Engineering, 2013; ‘Vol.’ 34, p 139.
86. Grenville, R. K.; Mak, A. T. C.; Brown, D. A. R. Suspension of solid particles in vessels agitated by axial flow impellers. Chemical Engineering Research and Design 2015, 100, 282-291.
87. Fentiman, N. J.; Lee, K. C.; Paul, G. R.; Yianneskis, M. On the Trailing Vortices Around Hydrofoil Impeller Blades. Chemical Engineering Research and Design 1999, 77, (8), 731-740.
88. Behafarid, F., Brasseur, J. G., Hydrodynamic Impacts on Dissolution, Transport and Absorption from Thousands of Drug Particles Moving within the Intestines. Bull. Amer. Phys. Soc., 2017.
89. Brasseur, J. G., Wang, Y., Hydrodynamic Enhancements of Dissolution from Drug Particles: In vivo vs. In vitro. Bull. Amer. Phys. Soc., 2013.
90. Plackett, R. L.; Burman, J. P. The Design of Optimum Multifactorial Experiments. Biometrika 1946, 33, (4), 305-325.
91. Stowe, R. A.; Mayer, R. P. EFFICIENT SCREENING OF PROCESS VARIABLES. Industrial & Engineering Chemistry 1966, 58, (2), 36-40.
92. Vanaja, K.; Shobha Rani, R. H. Design of Experiments: Concept and Applications of Plackett Burman Design. Clinical Research and Regulatory Affairs 2007, 24, (1), 1-23.

Each of the references listed above and cited throughout the disclosure is incorporated by reference herein in its entirety, or in relevant part, as would be apparent from context. The disclosed subject matter has been described with reference to various specific embodiments and techniques. It should be understood, however, that many variations and modifications may be made while remaining within the spirit and scope of the disclosed subject matter.

What is claimed is:

1. An in vitro method of measuring absorption of an orally administrable compound as a method of assessing the absorption of the compound in the vertebrate gastrointestinal tract, the method comprising:
(a) contacting a silicone-based polymer with an orally administrable compound in vitro; and
(b) measuring the absorption rate of the compound.

2. The method of claim 1 wherein the polymer is a poly (dimethyl siloxane), a poly di-methyl silicone or a poly siloxane polymer.

3. The method of claim 2 wherein the polymer is a poly (di-methyl siloxane) (PDMS) polymer.

4. The method of claim 1 wherein the absorption measure comprises:
(a) determining the aqueous initial concentration of compound before exposure to the polymer;
(b) measuring the rate of appearance of compound after exposure to the polymer in a receiver compartment; and
(c) using a scaled surface area of the polymer and scaled volume available for diffusion to assess the absorption of the compound in the vertebrate gastrointestinal tract.

5. The method of claim 1 wherein the polymer comprises pores having an average pore diameter of 0.4 to 0.9 nanometers.

6. The method of claim 5 wherein the pore diameter is 0.8 to 0.9 nanometers.

7. The method of claim 1 wherein the polymer has an average molecular weight between 6,000 and 70,000 daltons.

8. The method of claim 1 wherein the polymer is derivatized with end groups comprising at least one methyl end group, at least one hydroxyl end group, at least one vinyl end group, or at least one hydrogen end group, wherein the polymer is derivatized with an end group at each end of the polymer.

9. The method of claim 1 wherein the compound is hydrophilic.

10. The method of claim 1 wherein the compound is hydrophobic.

11. The method of claim 1 wherein the compound is negatively charged.

12. The method of claim 1 wherein the compound is positively charged.

13. The method of claim 1 wherein the compound is uncharged.

14. The method of claim 1 wherein the compound is a Biopharmaceutics Classification System (BCS) Class I or Class II compound exhibiting high permeability.

15. The method of claim 1 wherein the compound is a Biopharmaceutics Classification System (BCS) Class III or Class IV compound exhibiting low permeability.

16. The method of claim 1 wherein the polymer comprises pores stable in size for at least 193 days.

17. The method of claim 1 wherein the polymer exhibits an elastic modulus of at least 0.2 MPa.

18. The method of claim 1 wherein the polymer exhibits an elastic modulus no greater than 2.50 MPa.

19. The method of claim 1 wherein the polymer comprises a cross-linking agent between 3% and 25% weight percent.

20. The method of claim 1 wherein the polymer is in the form of a membrane.
